(19) United States
(12) Patent Application Publication
(10) Pub. No.: US 2004/0082642 A1
(43) Pub. Date: Apr. 29, 2004

(54) Novel estrogen receptor ligands and method III

(76) Inventors: Konrad Koehler, Huddinge (SE); Mikael Gillner, Stockholm (SE); Ye Liu, Tullinge (SE); Aiping Cheng, Huddinge (SE); James V Heck, Rahway, NJ (US); Milton Lloyd Hammond, Somerville, NJ (US); Ralph Troy Mosley, Rahway, NJ (US); Mahmoud Rahimi-Ghadim, Stockholm (SE)

Correspondence Address: WIGGIN & DANA LLP, ATTENTION: PATENT DOCKETING, ONE CENTURY TOWER, P.O. BOX 1832, NEW HAVEN, CT, 06508-1832, US

(21) Appl. No.: 10/433,271

(22) PCT Filed: Nov. 28, 2001
(86) PCT No.: PCT/EP01/13722

(30) Foreign Application Priority Data
Dec. 8, 2000 (GB) 0030037.6

(51) Int. Cl. C07D 209/54; A61K 31/407

(52) U.S. Cl. 514/409; 548/408

(57) ABSTRACT

The present invention relates to compounds of formula (I) and derivatives thereof, in which the variables are as defined in the claims, their synthesis, and their use as estrogen receptor modulators. The compounds of the instant invention are ligands for estrogen receptors and as such may be useful for treatment or prevention of a variety of conditions related to estrogen functioning including bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restinosis, gynecomastia, autoimmune disease, vascular smooth muscle cell profileration, obesity, incontinence, and cancer of the lung, colon, breast, uterus, and prostate.

### FIELD OF INVENTION

This invention relates to novel compounds which are estrogen receptor ligands and are preferably selective for either the estrogen receptor α or β isoforms, to methods of preparing such compounds and to methods for using such compounds such as for estrogen hormone replacement therapy and for diseases modulated by the estrogen receptor such as osteoporosis, elevated blood triglyceride levels, atherosclerosis, endometriosis, cognitive disorders, urinary incontinence, autoimmune disease, and cancer of the lung, colon, breast, uterus and prostate.

### BACKGROUND OF THE INVENTION

The estrogen receptor (ER) is a ligand activated mammalian transcription factor involved in the up and down regulation of gene expression. The natural hormone for the estrogen receptor is β-17-estradiol (E2) and closely related metabolites. Binding of estradiol to the estrogen receptor causes a dimerization of the receptor and the dimer in turn binds to estrogen response elements (ERE's) on DNA. The ER/DNA complex recruits other transcription factors responsible for the transcription of DNA downstream from the ERE into mRNA which is eventually is translated into protein. Alternatively the interaction of ER with DNA may be indirect through the intermediacy of other transcription factors, most notably fos and jun. Since the expression of a large number of genes is regulated by the estrogen receptor and since the estrogen receptor is expressed in many cell types, modulation of the estrogen receptor through binding of either natural hormones or synthetic ER ligands can have profound effects on the physiology and pathophysiology of the organism.

Estrogens are critical for sexual development in females. In addition, estrogens play an important role in maintaining bone density, regulation of blood lipid levels, and appear to have neuroprotective effects. Consequently decreased estrogen production in post-menopausal women is associated with a number of diseases such as osteoporosis, atherosclerosis, and cognitive disorders. Conversely certain types of proliferative diseases such as breast and uterine cancer and endometriosis are stimulated by estrogens and therefore antiestrogens (i.e., estrogen antagonists) have utility in the prevention and treatment of these types of disorders.

In addition to women suffering from breast cancer, men afflicted with prostatic cancer can also benefit from anti-estrogen compounds. Prostatic cancer is often endocrine sensitive and androgen stimulation fosters tumor growth, while androgen suppression retards tumor growth. The administration of estrogen is helpful in the treatment and control of prostatic cancer because estrogen administration lowers the level of gonadotropin and consequently androgen levels.

The use of natural and synthetic estrogens in hormone replacement therapy has been shown to markedly decrease the risk of osteoporosis. In addition, there is evidence that hormone replacement therapy has cardiovascular and neuroprotective benefits. However hormone replacement therapy is also associated with an increase risk of breast and uterine cancer. It is known that certain types of synthetic ER ligands display a mixed agonist/antagonist profile of activity showing agonist activity in some tissues and antagonist activity in other tissues. Such ligands are referred to as selective estrogen receptor modulators (SERMS). For example tamoxifen and raloxifene are known to be agonists in bone (and therefore prevent osteoporosis) while displaying antagonistic properties in breast (and therefore lowers the risk of breast cancer). However neither tamoxifen nor raloxifene is ideal for hormone replacement therapy as neither of these SERMS are as efficacious as estradiol in preventing bone loss. Furthermore the use of tamoxifen is still associated with an increased risk of uterine cancer and both tamoxifen and raloxifene are known to aggravate hot flashes.

Historically it has been believed there was only one estrogen receptor. However recently a second subtype (ER-β) has been discovered. While both the “classical” ER-α and the more recently discovered ER-β are widely distributed in different tissues, they nevertheless display markedly different cell type and tissue distributions. Therefore synthetic ligands which are either ER-α or ER-β selective may preserve the beneficial effects of estrogen while reducing the risk of undesirable side effects.

What is needed in the art are compounds that can produce the same positive responses as estrogen replacement therapy without the negative side effects. Also needed are estrogen-like compounds that exert selective effects on different tissues of the body.

The compounds of the instant invention are ligands for estrogen receptors and as such may be useful for treatment or prevention of a variety of conditions related to estrogen functioning including bone loss, bone fractures, osteoporosis, cartilage degeneration, endometriosis, uterine fibroid disease, hot flashes, increased levels of LDL cholesterol, cardiovascular disease, impairment of cognitive function, cerebral degenerative disorders, restinosis, gynecomastia, vascular smooth muscle cell proliferation, obesity, incontinence, autoimmune disease and cancer of the lung, colon, breast, uterus, and prostate.

### DESCRIPTION OF INVENTION

In accordance with the present invention, compounds are provided which are estrogen receptor ligands and have the general formula I:

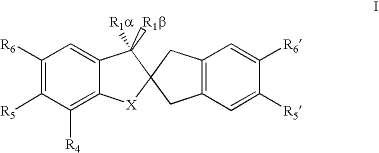

wherein R₁α and R₁β may together be a single nitrogen atom which is in turn bonded a, group selected from R^{A} or OR^{A}; or R₁α and R₁β may together be a single carbon atom which in turn is bonded to two R^{A} groups which may be the same or are different; or R₁α and R₁β are the same or are different and selected from hydroxyl, R^{A} or OR^{A},

R^{A} is selected from hydrogen, alkyl, alkenyl, alkynyl, cycloalkyl, cycloalkylalkyl, aryl or arylalkyl, provided that R₁α and R₁β are not both H, R₁α is not OH when R₁β is H, and R₁β is not OH when R₁α is H,

X is a methylene group (—CH₂—), an ethylene group (—CH₂CH₂—), or a substituted methylene group (—CR^{B}H—) where R^{B} is a C₁-C₄ alkyl group,

R₄ is a hydrogen atom, or an alkyl group of 1 to 4 carbon atoms, or a halogen atom,

R₅, R₆, R_{5′}, and R_{6′} are the same or are different and are a hydrogen atom, or hydroxyl group, or an alkyloxy group of 1 to 4 carbon atoms, or an acyloxy group of 1 to 4 carbon atoms, or an aminoalkoxy group, or a halogen atom;

and pharmaceutically acceptable salts and stereoisomers thereof.

### DETAILED DESCRIPTION OF INVENTION

The present invention relates to compounds useful as estrogen receptor modulators and have the general formula I as described above.

One embodiment of the present invention relates compounds according to the general formula II, wherein at least one of the R₅ or R₆ substituents is a hydrogen atom and at least one of the R_{5′} or R_{6′} substituents is also a hydrogen atom.

One class of this embodiment relates compounds according to the general formula I, wherein at least one of R₅, R₆, R_{5′}, or R_{6′} is a group selected from hydroxyl, acyloxy, chlorine, or bromine.

Another class of this embodiment relates compounds according to the general formula I, wherein the remaining substituents R₅, R₆, R_{5′}, or R_{6′} are the same or are different and selected from the group hydroxyl or acyloxy.

Yet another class of this embodiment relates compounds according to the general formula I, wherein one of the remaining substituents R₅, R₆, R_{5′}, or R_{6′} is hydroxyl or acyloxy and the other remaining substituent is aminoalkoxy as herein defined.

Another embodiment of the present invention relates compounds according to the general formula I, wherein one of R₁α and R₁β is selected from the group hydrogen or methyl or hydroxyl and the other is selected from the group n-propyl, 2-propenyl, 2-propynyl, n-butyl, 2-butenyl, 3-butenyl, 2-butynyl, 3-butynyl, n-pentyl, 3-methylbutyl, 3-methyl-1-butenyl, 3-methyl-2-butenyl, 3-methylpentyl, 3-ethylpentyl, cyclopropylethyl, cyclopentylethyl, cyclohexylethyl, cycloheptylethyl, cyclopropylpropyl, cyclopentylpropyl, benzyl, or phenethyl.

One class of this embodiment relates compounds according to the general formula I, wherein X is a methylmethylene group [—C(CH₃)H—].

Another embodiment of the present invention relates compounds according to the general formula I, wherein R₁α and R₁β may together be a single carbon atom (i e., an exo methylene carbon atom) which in turn is bonded to two groups R^{C} and R^{D}, wherein R^{C} is selected from the group hydrogen or methyl and RD is selected from the group aryl, benzyl, ethyl, n-propyl, i-propyl, 2-propenyl, 2-propynyl, n-butyl, 2-butenyl, 3-butenyl, 2-butynyl, 3-butynyl, 2-methylbutyl, 2-methyl-1-butenyl, 2-methyl-2-butenyl, cyclopropylmethyl, cyclopentylmethyl, cyclohexylmethyl, or cycloheptylmethyl.

One class of this embodiment relates compounds according to the general formula I, wherein X is a methylmethylene group [(—CH₃)H—].

Another embodiment of the present invention relates compounds according to the general formula II or III:

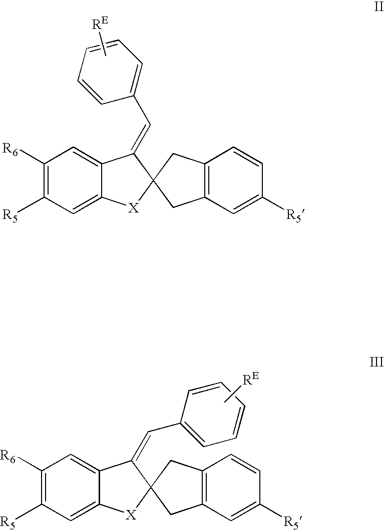

wherein X is a methylene group (—CH₂—) or an ethylene group (—CH₂CH₂—), one of R₅ or R₆ is a hydrogen atom and the other is a hydroxyl or acyloxy group, one of R_{5′} and R^{E} is selected from the group hydroxyl, acyloxy, methoxy, or ethoxy and the other is selected from the group aminoalkoxy;

and pharmaceutically acceptable salts or stereoisomers thereof.

Compounds of the invention include, but are not limited to, the following:

Anti-5,5′-dihydroxy-1,1′,3,3′-tetrahydro-2,2′-spirobi(2H-indene)-1-one-(N-methyl oxime) (E9a);

Syn-5,5-dihydroxy-1,1′,3,3′-tetrahydro-2,2′-spirobi(2H-indene)-1-one-(N-methyl oxime)(E9b);

5,5′-Dihydroxy-1,1′,3,3′-tetrahydro-2,2′-spirobi(2H-indene)-1-one-oxime (E10a);

5′-Hydroxy-5-methoxy-1,1′,3,3′-tetrahydro-2,2′-spirobi(2H-indene)-1-one-oxime (E10b);

5,5′-Dihydroxy-1,1′,3,3′-tetrahydro-2,2′-spirobi(2H-indene)-1-methylidene (E11);

5,5′-Dihydroxy-1-methyl-1,1′,3,3′-tetrahydro-2,2′-spirobi(2H-indene) (E12);

1-Butyl-5,5′-hydroxy-1,1′,3,3′-tetrahydro-2,2′-spirobi(2H-indene) (E13);

5-Hydroxy-5′-(2″-piperidinylethoxy)-1-(p-methoxy)benzylidene-1,1′,3,3′-tetrahydro-2,2′-s pirobi(2H-indene) (E14a);

6-Hydroxy-5′-(2″-piperidinylethoxy)-1-(p-methoxy)benzylidene-1,1′,3,3′-tetrahydro-2,2′-s pirobi(2H-indene) (E14b);

Z-5-Hydroxy-5′-(2″-piperidinylethoxy)-1-(m-methoxy)benzylidene-1,1′,3,3′-tetrahydro-2,2′-spirobi(2H-indene) (E14c);

Z-5-Hydroxy-5′-(2′-piperidinylethoxy)-1-(m-hydroxy)benzylidene-1,1′,3,3′-tetrahydro-2,2′-spirobi(2H-indene) (E14d);

5,5′-Dihydroxy-1,3-dimethyl-1,1′,3,3′-tetrahydro-2,2′-spirobi(2H-indene) (E18);

5,5′-Dihydroxy-1-ethyl-3-methyl-1,1′,3,3′-tetrahydro-2,2′-spirobi(2H-indene) (E19);

5,5′-Dihydroxy-1-propyl-3-methyl-1,1′,3,3′-tetrahydro-2,2′-spirobi(2H-indene) (E20);

6,5′-Dihydroxy-1-methyl-1,1′,3,3′-tetrahydro-2,2′-spirobi(2H-indene) (E23);

6,5′-Dihydroxy-1-ethyl-1,1′,3,3′-tetrahydro-2,2′-spirobi(2H-indene) (E24);

6,5′-Dihydroxy-1-butyl-1,1′,3,3′-tetrahydro-2,2′-spirobi(2H-indene) (E25);

6,5′-Dihydroxy-1-benzylidene-1,1′,3,3′-tetrahydro-2,2′-spirobi(2H-indene) (E26);

6′,5′-Dihydroxy-1-(p-methoxy)benzylidene-1,1′,3,3′-tetrahydro-2,2′-spirobi(2H-indene) (E27);

6,5′-Dihydroxy-1-(p-hydroxy)benzylidene-1,1′,3,3′-tetrahydro-2,2′-spirobi(2H-indene) (E28);

Rac-(1′R,2S/1′S,2R)-6,5′-dihydroxy-1-(p-methoxy)benzyl-1,1′,3,3′-tetrahydro-2,2′-spirobi(2H-indene) (E29a);

rac-(1′R,2R/1′S,2S)-6,5′-Dihydroxy-1-6methoxy)benzyl-1,1′,3,3′-tetrahydro-2,2′-spirobi(2H-indene) (E29b);

6,5′-Di[(t-butyldimethyl)silyloxy]-1-[4-benzyloxy(benzylidene)]-1,1′,3,3′-tetrahydro-2,2′-spirobi(2H-indene) (E30);

6,5′-Dihydroxy-1-(p-benzyloxy)benzylidene-1′,3,3′-tetrahydro-2,2′-spirobi(2H-indene) (E31)

rac-(1′R,2S/1′S,2R)-6,5′-Dihydroxy-1-[p-(2″-piperidinylethoxy)benzyl]-1,1′,3,3′-tetrahydro-2,2′-spirobi(2H-indene) (E32a);

rac-(1′R,2R/1′S,2S)-6,5′-Dihydroxy-1-[p-(2″-piperidinylethoxy)benzyl]-1,1′,3,3′-tetrahydro-2,2′-spirobi(2H-indene) (E32b);

5,7′-Dihydroxy-1′-methyl-1,3,3′,4′-tetrahydro-spiro[2H-indene-2,2′-(1′H)-naphthalene] (E38);

5,6′-Dihydroxy-1′-methyl-1,3,3′,4′-tetrahydro-spiro[2H-indene-2,2′-(1′H)-naphthalene] (E44);

5,6′-Dihydroxy-1′-ethylidene-1,3,3′,4′-tetrahydro-spiro[2H-indene-2,2′-(1′H)-naphthalene] (E45a);

5,6′-Dihydroxy-1′-isopropylidene-1,3,3′,4′-tetraiydro-spiro[2H-indene-2,2′-(1′H)-naphthalene] (E45b);

(Z)-5,6′-Dihydroxy-1′-propylidene-1,3,3′,4′-tetrahydro-spiro[2H-indene-2,2′-(1′H)-naphthalene] (E45c);

(E)-5,6′-Dihydroxy-1′-propylidene-1,3,3′,4′-tetrahydro-spiro[2H-indene-2,2′-(1′H)-naphthalene] (E45d);

(1R,2S)- and (1S, 2R)-5,1′,6′-Trihydroxy-1′-phenyl-1,3,3′,4′-tetrahydro-spiro[2H-indene-2,2′-(1′H)-naphthalene] (E45e);

(1R,2R)- and (1S,2S)-5,1′,6′-Trihydroxy-1′-phenyl-1,3,3′,4′-tetrabydro-spiro[2H-indene-2,2′-(1′H)-naphthalene] (E45f);

5,6′-Dihydroxy-1′-(p-methoxy)benzylidene-1,3,3′,4′-tetrahydro-spiro[2H-indene-2,2′-(1′H)-naphthalene] (F[46);

and pharmaceutically acceptable salts and stereoisomers thereof.

Another embodiment of the invention is a method of eliciting an estrogen receptor modulating effect in a mammal in need thereof, comprising administering to the mammal a therapeutically effective amount of any of the compounds or any of the pharmaceutical compositions described above.

A class of the embodiment is the method wherein the estrogen receptor modulating effect is an agonizing effect.

A subclass of the embodiment is the method wherein the estrogen receptor is an ERα receptor.

A second subclass of the embodiment is the method wherein the estrogen receptor is an ERβ receptor.

A third subclass of the embodiment is the method wherein the estrogen receptor modulating effect is a mixed ERα and ERβ agonizing effect.

A second class of the embodiment is the method wherein the estrogen receptor modulating effect is an antagonizing effect.

A subclass of the embodiment is the method wherein the estrogen receptor is an ERα receptor.

A second subclass of the embodiment is the method wherein the estrogen receptor is an ERβ receptor.

A third subclass of the embodiment is the method wherein the estrogen receptor modulating effect is a mixed ERα and ERβ antagonizing effect.

Another embodiment of the invention is a method of treating or preventing hot flashes in a mammal in need thereof by administering to the mammal a therapeutically effective amount of any of the compounds or pharmaceutical compositions described above.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

As a specific embodiment of this invention, 32 mg of 5,5′-Dihydroxy-1-propyl-3-methyl-1,1′,3,3′-tetrahydro-2,2′-spirobi(2H-indene) from Example 20, is formulated with sufficient finely divided lactose to provide a total amount of 580 to 590 mg to fill a size 0, hard-gelatine capsule.

Further exemplifying the invention is the use of any of the compounds described above in the preparation of a medicament for the treatment and/or prevention of osteoporosis in a mammal in need thereof. Still further exemplifying the invention is the use of any of the compounds desribed above in the preparation of a medicament for the treatment and/or prevention of bone loss, bone resorption, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restinosis, gynecomastia, vascular smooth muscle cell proliferation, obesity, incontinence, autoimmune disease, lung cancer, colon cancer, breast cancer, uterine cancer, prostate cancer, and/or disorders related to estrogen functioning.

The present invention is also directed to combinations of any of the compounds or any of the pharmaceutical compositions described above with one or more agents useful in the prevention or treatment of osteoporosis. For example, the compounds of the instant invention may be effectively administered in combination with effective amounts of other agents such as an organic bisphosphonate or a cathepsin K inhibitor. Nonlimiting examples of said organic bisphosphonates include adendronate, clodronate, etidronate, ibandronate, incadronate, minodronate, neridronate, risedronate, piridronate, pamidronate, tiludronate, zoledronate, pharmaceutically acceptable salts or esters therof, and mixtures thereof. Preferred organic biphosphonate include alendronate and pharmaceutically acceptable salts and mixtures thereof. Most preferred is alendronate monosodium trihydrate.

The precise dosage of the bisphonate will vary with the dosing schedule, the oral potency of the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. An appropriate amount can be determined by routine experimentation from animal models and human clinical studies. Generally, an appropriate amount of bisphosphonate is chosen to obtain a bone resorption inhibiting effect, i.e. a bone resorption inhibiting amount of the bisphonsphonate is administered. For humans, an effective oral dose of bisphosphonate is typically from about 1.5 to about 6000 μg/kg of body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis, i.e. on the basis of the corresponding acid.

The compounds of the present invention can be used in combination with other agents useful for treating estrogen-mediated conditions. The individual components of such combinations can be administer separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating estrogen-mediated conditions includes in principle any combination with any pharmaceutical composition useful for treating disorders related to estrogen functioning.

The compounds of the present invention can be administered in such oral dosage forms as tablets, capsules (each of which includes sustained release or timed release formulations), pills, powder, granules, elixirs, tinctures, suspensions, syrups and emulsions. Likewise, they may also be administered in intravenous (bolus or infusion), intraperitoneal, topical (e.g., ocular eyedrop), subcutaneous, intramuscular, or transdermal (e.g., patch) form, all using forms well known to those of ordinary skill in the pharmaceutical arts.

The dosage regimen utilizing the compounds of the present invention is selected in accordance with a variety of factors including type, species, age, weight, sex, and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches will known to those of ordinary skill in the art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, exipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms includes sodium oleate, sodium stearate, magnesium stearate, sodium benzoate, sodium acetate, sodium chloride and the like. Disintegrators include without limitation starch, methylcellulose, agar, bentonite, xanthan gum and the like.

The compounds of the present invention can also be administered in the form of liposome delivery systems, such as small unilamellar vesicles, large unilamellar vesicles and multilamellar vesicles. Liposomes can be formed form a variety of phospholipids, such as 1,2-dipalmitoylphosphatidylcholine, phosphatidyl ethanolamine(cephahine), or phosphatidylcholine (lecithin).

The following definitions apply to the terms as used throughout this specification, unless otherwise limited in specific instances.

The term “estrogen receptor ligand” as used herein is intended to cover any moiety which binds to a estrogen receptor. The ligand may act as an agonist, an antagonist, a partial agonist or a partial antagonist. The ligand may be either ERα or ERβ selective or display mixed ERα and ERβ activity.

The term “aliphatic hydrocarbon(s)” as used herein refers to acyclic straight or branched chain groups which include alkyl, alkenyl or alkynyl groups.

The term “aromatic hydrocarbon(s)” as used herein refers to groups including aryl groups as defined herein.

Unless otherwise indicated, the term “lower alkyl”, “alkyl” or “alk” as employed herein alone or as part of another group includes both straight and branched chain hydrocarbons, containing 1 to 12 carbon atoms (in the case of alkyl) in the normal chain and preferably 1 to 6 carbons, such as methyl, ethyl, propyl, isopropyl, butyl, t-butyl, or isobutyl, pentyl, hexyl, isohexyl, heptyl, 4,4-dimethylpentyl, octyl, 2,2,4-trimethylpentyl, nonyl, decyl, undecyl, dodecyl.

The term “cycloalkyl” as employed herein alone or as part of another group refers to 3- to 7-membered fully saturated mono cyclic ring system and include cyclopropyl, cyclobutyl, cyclopentyl, cyclohexyl, and cycloheptyl.

The term “cycloalkylalkyl” as employed herein alone or as part of another group refers to an cycloalkyl group containing 3 to 7 carbon atoms attached through available carbon atoms to a straight or branched chain alkyl radical containing 1 to 6 carbon atoms and include but are not limited to cyclopropylmethyl (—CH₂C₃H₅), cyclobutylethyl (—CH₂CH₂C₄H₇), and cyclopentylpropyl (—CH₂CH₂CH₂C₅H₉).

Unless otherwise indicated, the term “lower alkenyl” or “alkenyl” as used herein by itself or as part of another group refers to straight or branched chain radicals of 2 to 12 carbons, preferably 2 to 6 carbons, in the normal chain, which include one to six double bonds in the normal chain, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, and the like.

Unless otherwise indicated, the term “lower alkynyl” or “alkynyl” as used herein by itself or as part of another group refers to straight or branched chain radicals of 2 to 12 carbons, preferably 2 to 6 carbons, in the normal chain, which include one triple bond in the normal chain, such as 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, 3-nonynyl, 4-decynyl, 3-undecynyl, 4-dodecynyl and the like.

The term “halogen” or “halo” as used herein alone or as part of another group refers to chlorine, bromine, fluorine, and iodine as well as CF₃.

The term “akyloxy” as employed herein alone or as part of another group refers to an oxygen atom which is in turn bonded to a linear or branched alkyl group of from 1 to 4 carbon atoms and includes but is not limited to methoxy (—OCH₃), ethoxy (—OCH₂CH₃), butoxy (—OCH₂CH₂CH₂CH₃), and isopropoxy [—OCH₂(CH₂)CH₃].

The term “acyloxy” as employed herein alone or as part of another group refers to an oxygen atom which is in turn bonded to carbonyl group (C═O) which is in turn bonded a linear or branched alkyl group of from 1 to 4 carbon atoms and includes but is not limited to acetoxy [—O(C═O)CH₃], propionyloxy [—O(C—)CH₂CH₃], and butyryloxy [—O(C═O)CH₂CH₂CH₃].

The term “linking alkyl” as employed herein alone or as part of another group refers to a linear bivalent radical hydrocarbon chain of from 0 to 6 carbon atoms in which the terminal carbon atoms are radicals (formed by removal of a hydrogen atom) and include 0 (a bond), 1 (methylene, —CH₂—), 2 ethylene (—CH₂CH₂—), and 3 (trimethylene, —CH₂CH₂CH₂—) carbon atom chains.

The term “aminoalkoxy” as employed herein alone or as part of another group refers to an oxygen atom which is in turn bond to a linking allyl group of from 2 to 3 carbon atoms which is in turn bonded to a primary, secondary, or tertiary amine (—NR₁R₂). In the amine proportion of the aminoalkoxy group (—NR₁R₂), R₁ and R₂ are the same or are different and are a hydrogen atom, or a linear or branched alkyl group of from 14 carbon atoms, or an aryl group; or R₁ and R₂ are the same and are a linking alkyl group of 3-6 carbon atoms; or R₁ and R₂ are the same and is an ethyloxyethyl diradical group (—CH₂CH₂OCH₂CH₂—). Aminoalkoxy groups include but are not limited to 2-aminoethoxy (—OCH₂CH₂NH₂), 3-aminopropoxy (—OCH₂CH₂CH₂NH₂), 2-(N,N-diethylamino)ethoxy (—OCH₂CH₂N(t)₂), 2-(1-piperidinyl)ethoxy (

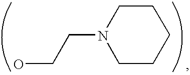

), and 2-(1-morpholinyl)ethoxy

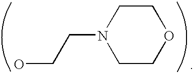

).

The term “aryl” as employed herein alone or as part of another group refers to monocyclic and bicyclic aromatic groups containing 6 to 10 carbons in the ring portion and may be optionally substituted through available carbon atoms with 1, 2, or 3 groups selected from hydrogen, halo, alkyl, haloalkyl, alkoxy, haloalkoxy, alkenyl, amino, trifluoromethyl, trifluoromethoxy, alkynyl, hydroxy, nitro, cyano, carboxy, or aminoalkoxy. Aryl groups include but are not limited to phenyl, 1-naphthyl, 2-naphthyl, 4-[2-(N,N-diethylaminoethoxy)]phenyl

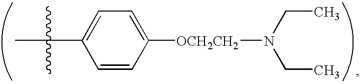

4-[2-(1-piperidinylethoxy)]phenyl]

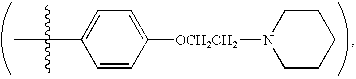

and 4-[2-(1-morpholinylethoxy)]phenyl

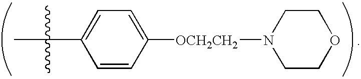

The term “arylalkyl” as employed herein alone or as part of another group refers to an aryl group containing 6 to 10 carbon atoms attached through available carbon atoms to a straight or branched chain alkyl radical containing 1 to 6 carbon atoms. The meta or para positions of the aromatic portion of the arylakyl group may be optionally substitued with an aminoalkoxy group. Arylalkyl groups include but are not limited to benzyl (—CH₂Ph), phenethyl (—CH₂CH₂Ph), phenpropyl (—CH₂CH₂CH₂Ph), 1-napthylmethylene (—CH₂C₁₀H₇), 4-[2-(N,N-diethylaminoethoxy)]benzyl (

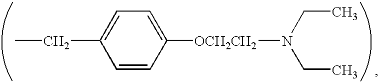

), 4-[2-(1-piperidinylethoxy)]benzyl] (

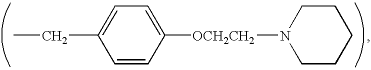

), and 4-[2-morpholinylethoxy)]benzyl (

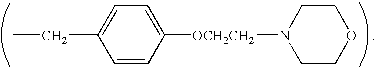

).

The compounds of formula I can be present as salts, in particular pharmaceutically acceptable salts. If the compounds of formula 1[have, for example, at least one basic center, they can form acid addition salts. These are formed, for example, with strong inorganic acids, such as mineral acids, for example sulfuric acid, phosphoric acid or a hydrohalic acid, with strong organic carboxylic acids, such as alkanecarboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such as hydroxycarboxylic acids, for example ascorbic, glycolic, lactic, malic, tartaric or citric acid, such as amino acids, (for example aspartic or glutamic acid or lysine or arginine), or benzoic acid, or with organic sulfonic acids, such as (C₁-C₄)-alkyl- or aryl-sulfonic acids which are unsubstituted or substituted, for example by halogen, for example methane- or p-toluene-sulfonic acid. Corresponding acid addition salts can also be formed having, if desired, an additionally present basic center. The compounds of formula I having at least one acid group (for example COOH) can also form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may furthermore be formed. Salts which are unsuitable for pharmaceutical uses but which can be employed, for example, for the isolation or purification of free compounds I or their pharmaceutically acceptable salts are also included.

Preferred salts of the compounds of formula I which include a basic group include monohydrochloride, hydrogensulfate, methanesulfonate, phosphate or nitrate.

Preferred salts of the compounds of formula I which include an acid group include sodium, potassium and magnesium salts and pharmaceutically acceptable organic amines.

The compounds in the invention contain at least one chiral center and therefore exist as optical isomers. The invention therefore comprises the optically inactive racemic (rac) mixtures (a one to one mixture of enantiomers), optically enriched scalemic mixtures as well as the optically pure individual enantiomers. The compounds in the invention also may contain more than one chiral center and therefore may exist as diastereomers. The invention therefore comprises individual diastereomers as well as mixtures of diastereomers in cases where the compound contains more than one stereo center. The compounds in the invention also may contain acyclic alkenes or oximes and therefore exist as either the E (entgegen) or Z (zusammen) isomers. The invention therefore comprises individual E or Z isomers as well as mixtures of E and Z isomers in cases where the compound contains an acylic alkene or oxime funtional group. Also included within the scope of the invention are polymorphs, hydrates, and solvates of the compounds of the instant invention.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the, treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example in “Design of Prodrugs” ed. H. Bundgaard, Elsevier, 1985, which is incorporated by reference herein in its entirety. Metabolites of the compounds includes active species produced upon introduction of compounds of this invention into the biological milieu.

The present invention also relates to pharmaceutical compositions comprising the compounds of the present invention and a pharmaceutically acceptable carrier.

The present invention also relates to methods for making the pharmaceutical compositions of the present invention.

The present invention also relates to methods for eliciting an estrogen receptor modulating effect in a mammal in need thereof by administration of the compounds and pharmaceutical compositions of the present invention.

The present invention also relates to methods for eliciting an estrogen receptor antagonizing effect in a mammal in need thereof by administration of the compounds and pharmaceutical compositions of the present invention.

The present invention also relates to methods for treating or preventing disorders elated to estrogen functioning, bone loss, bone fractures, osteoporosis, cartilage degeneration, endometriosis, uterine fibroid disease, autoimmune disease, lung, colon, breast, uterus, or prostate cancer, hot flashes, cardiovascular disease, impairment of cognitive function, cerebral degenerative disorders, restenosis, gynecomastia, vascular smooth muscle cell proliferation, obesity and incontinence in a mammal in need thereof by administering the compounds and pharmaceutical compositions of the present invention.

The present invention also relates to methods for reducing bone loss, lowering LDL cholesterol levels and eliciting a vasodilatory effect, in a mammal in need thereof by administering the compounds and pharmaceutical compositions of the present invention.

The novel compounds of the present invention can be prepared according to the procedure of the following Schemes and examples, using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variation of the conditions and processes of the following preparative procedures can be used to prepare these compounds. The compounds of the present invention are prepared according to the general methods outlined in Schemes 1-3, and according to the methods described. All temperatures are degrees Celsius unless otherwise noted. The following abbreviations, reagents, expressions or equipment, which are amongst those used in the descriptions below, are explained as follows: 20-25° C. (room temperature, r.t), molar equivalent (eq.), dimethyl formamide, (DMF) dichloromethane (DCM), ethyl acetate (EtOAc), tetrahydrofuran (THF), lithium diisopropylamide (LDA), methyl t-butyl ether (MTBE), rotating glass sheet coated with a silica gel-gypsum mixture used for chromatographic purification (chromatotron), preparative liquid chromatography with a C8 stationary phase and ammonium acetate acetonitrile-water buffer as mobile phase (PHPLC), gaschromatography mass spectroscopy (GC-MS), electrospray mass spectroscopy (ES-MS).

A general route for the construction of the spiro core structure is shown in Scheme 1. This methodology is based on the chemistry described by Sakata, et al., Bull. Chem. Soc. Jpn 1994, 67, 3067-3075. Instep I, substituted indanone or tetralone is alkylated by corresponding dibromide to form spiro compound 1. In step II, the ketone is reduced to methylene derivative 2 and followed by demethylation by BBr₃ in step III to give the phenolic compound 3. In step IV, the ketone functional group was derivatized to oxime or methyl oxime 4. Finally in step V, demethylation affords the corresponding free phenol 5.

Representative protocols for step I (method A), step II (method B), step III and V (method C) as well as step IV (method D) in Scheme 1 are as follows:

Method A:

To a solution of the ketone (1.0 eq.) and dibromide (1.0-1.1 eq.) in benzene was added, portion-wise while stirring, potassium t-butoxide (2-3 eq.) at room temperature. The mixture was then stirred at 20-45° C. (para-methoxy derivatives) or 100° C. (meta-methoxy derivatives) for 2-12 h before treated with 10% HCl. The organic materials were thereafter taken up in EtOAc. This solution was dried (anhydrous magnesium sulfate), filtered and concentrated. The resulting residue was purified by column chromatography to yield the product.

Method B:

A mixture of the ketone (1.0 eq.) and triethylsilane (2-3 eq.) in trifluoro acetic acid (TFA) was stirred at room temperature for 2-4 days. TFA was removed by evaporation under vacuum. The resulting oil was partitioned between EtOAc and saturated sodium bicarbonate (aq.). The organic phase was washed with brine, dried (anhydrous magnesium sulfate), filtered and concentrated to give the crude product.

Scheme 1: A general route to spiro core structure and initial modification.

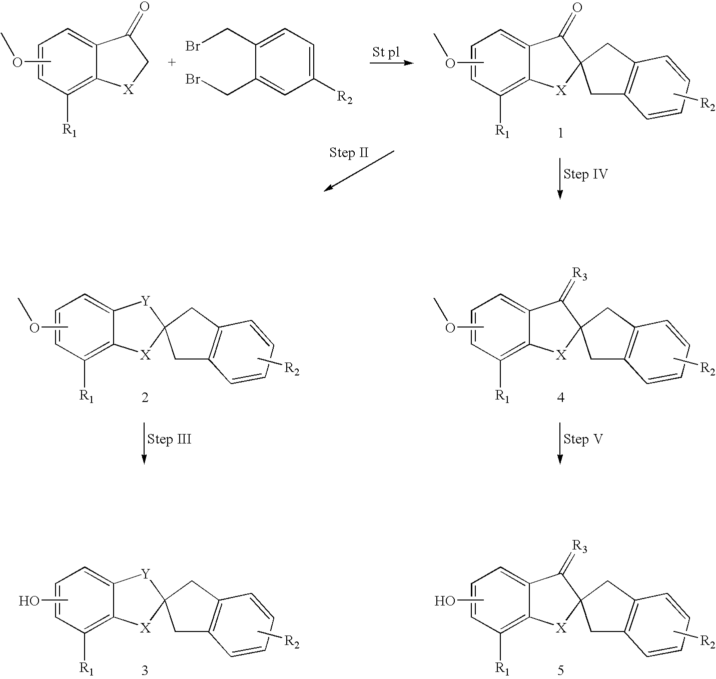

Deprotection (i.e., demethylation) can be done by two different known methods depending on the nature of the substrates, which only differs in reagent [BBr₃ or BF₃.(CH₃)S]. For a representive protocol see, method C).

Method C:

To a cool (dry-ice/acetone bath) and stirred solution of the aryl methyl ether (1.0 eq.) in dry DCM was added BBr₃ (1.0 M sol in DCM) or BF₃o(CH₃)₂S. The mixture was then allowed to come to 0° C. or room temperature, and kept at that temperature for the time indicated before quenching with ice-water. The organic materials were then taken up in EtOAc. This solution was dried (anhydrous magnesium sulfate) filtered and concentrated in vacuo to furnish the crude product.

Method D:

A mixture of hydroxyamine (or methoxyamine) hydrochloride (10 eq.) and sodium acetate (10 eq.) was dissolved in methanol and filtered after 5 min. The resulting solution was added into a mixture of the ketone (1.0 eq.) and molecular sieves (4 Å) in methanol. The mixture was stirred at 75-80° C. for 4 hours to 2 days. The organic materials were thereafter taken up in EtOAc. This solution was dried (anhydrous magnesium sulfate) filtered and concentrated in vacuo to furnish the crude product.

Scheme 2: Two major modifications of the spiro analog: modifying 1′-carbonyl or 5-hydroxy group.

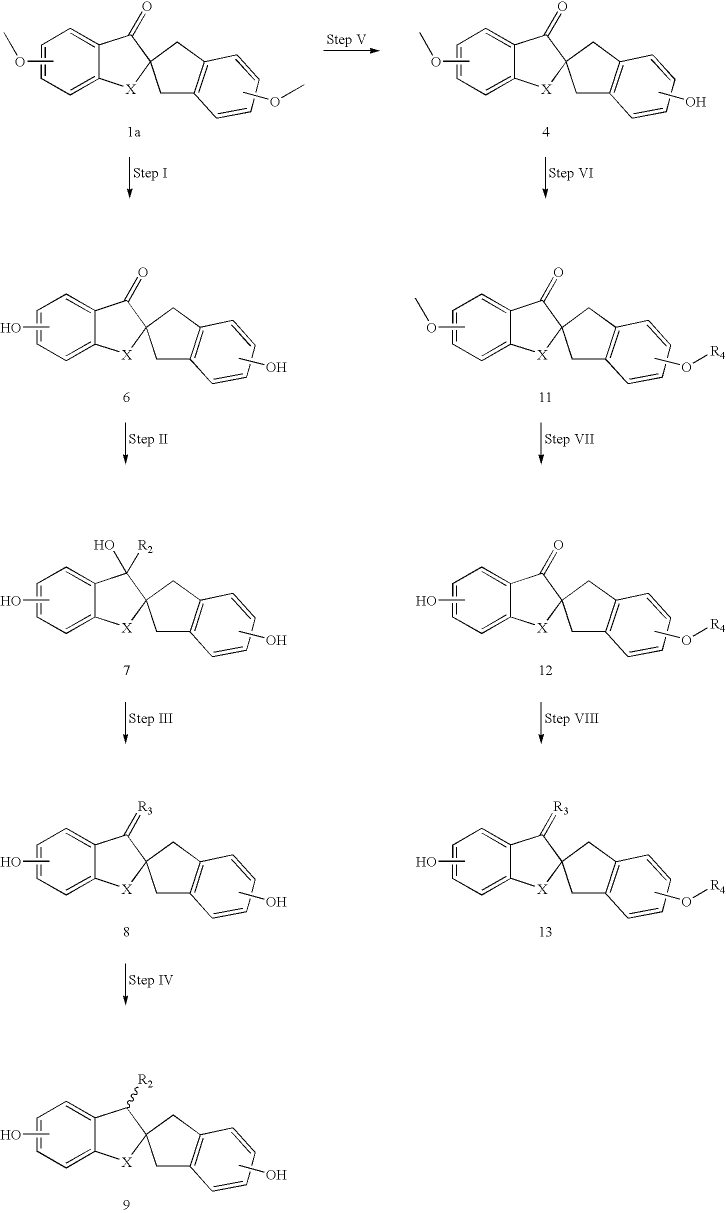

The dimethoxy ketone 1a can be further modified by employment of known methods for the reactions of 1′-carbonyl functionalities as well as the O-alkylations on the 5-hydroxy group as shown in Scheme 2. In step I, demethylation is carried out according to methods C to give the dihydroxy ketone 6. The compound 6 was treated with Grignard reagent in step II to generate the corresponding alcohol 7. The alcohol was isolated only in a few cases for screening and characterization. In most of the cases, the alcohol 7 was immediately converted to olefin 8 by acid catalyzed dehydration as shown in step III. In step IV, the double bond is reduced by catalytic hydrogenation to furnish 9. The selective mono-demethylation can be accomplished by controlling reaction temperature below −23° C. with BBr₃ as shown in step V and the resulting free phenol 10 was alkylated in step VI to give the aminoalkoxy derivative 11. In step VII, demethylation is repeated on the second methoxy group by standard condition (method C), affording phenol 12. In last step, the carbonyl functionality of 12 is modified in a similar way as method E.

Representative protocols for step II and III (method E), step IV (method F), step V (method G), step VI (method H), as well as step VII (method I) in Scheme 2 are as follows:

Method E:

To a cool (dry ice/acetone bath) solution of the ketone (1.0 eq.) in anhydrous THF was added while stirring, a solution of freshly prepared (or commercially available) Grignard reagent (large excess, at least 3 eq.) in anhydrous THF. The mixture was then allowed to stand at room temperature overnight. Quenching with saturated ammonium chloride (aq.) at 0° C. if alcohol is the desired product. In other cases when olefin is desired, 10% HCl is added and the mixture is stirred at room temperature for the time indicated. The organic materials were thereafter taken up in EtOAc. This solution was dried (anhydrous magnesium sulfate), filtered and concentrated in vacuo, to yield the crude product.

Method F:

A mixture of the olefin (1.0 eq.) and catalytic amount of PtO₂ in EtOAc was stirred under hydrogen from balloon at room temperature for the time indicated. The catalyst is removed by filtration through celite® and the filtrate is concentrated to furnish the crude product. As an alternative, Method K is also used for reduction of the olefin in some examples.

Method G:

To a cool (dry-ice/acetone bath) and stirred solution of the aryl methyl ether (1.0 eq.) in dry DCM was added BBr₃ (1.0 M sol in DCM). The mixture was then allowed to stand at −23° C. for the time indicated before quenching with ice-water. The organic materials were then taken up in EtOAc. This solution was dried (anhydrous magnesium sulfate) filtered and concentrated in vacuo to furnish the crude product.

Method H:

A mixture of the phenol (1.0 eq.), N-(2-chloroethyl)-piperidin hydrochloride (4.0 eq.) and potassium carbonate (4.0 eq.) in acetonitrile was stirred under reflux for 1 day. The mixture was partitioned between EtOAc and water. The organic phase was dried and concentrated to afford the crude product.

Method I:

To a cool (dry ice/acetone bath) solution of freshly prepared Grignard reagent (large excess, up to 10 eq.) in anhydrous THF was added while stirring, a solution of the ketone (1.0 eq.) in anhydrous THF. The mixture was then stirred at room temperature overnight. After stirring with 10% HCl for 2 hours, the reaction mixture was first treated with sodium bicarbonate until pH=8 and then extracted with EtOAc. The organic solution was dried (Na₂SO₄), filtered and concentrated in vacuo, to yield the crude product.

In step I of Scheme 3, the two hydroxyl groups of 8a are protected by treatment with tert-butyldimethylchlorosilane to yield the silyl ether 14. Both deprotection of the benzyloxy protecting group and reduction of the double bond are accomplished by palladium catalyzed hydrogenation to give monophenol 15 in step II and triol 18 in step V. A Mitsunobu reaction is performed in step III to obtain aminoethyloxy compound 16. Removal of the silyl protecting groups complete the synthesis to give final product 17 as shown in step IV.

Scheme 3: Further modifications of the 1′-benzylidene derivative 8a.

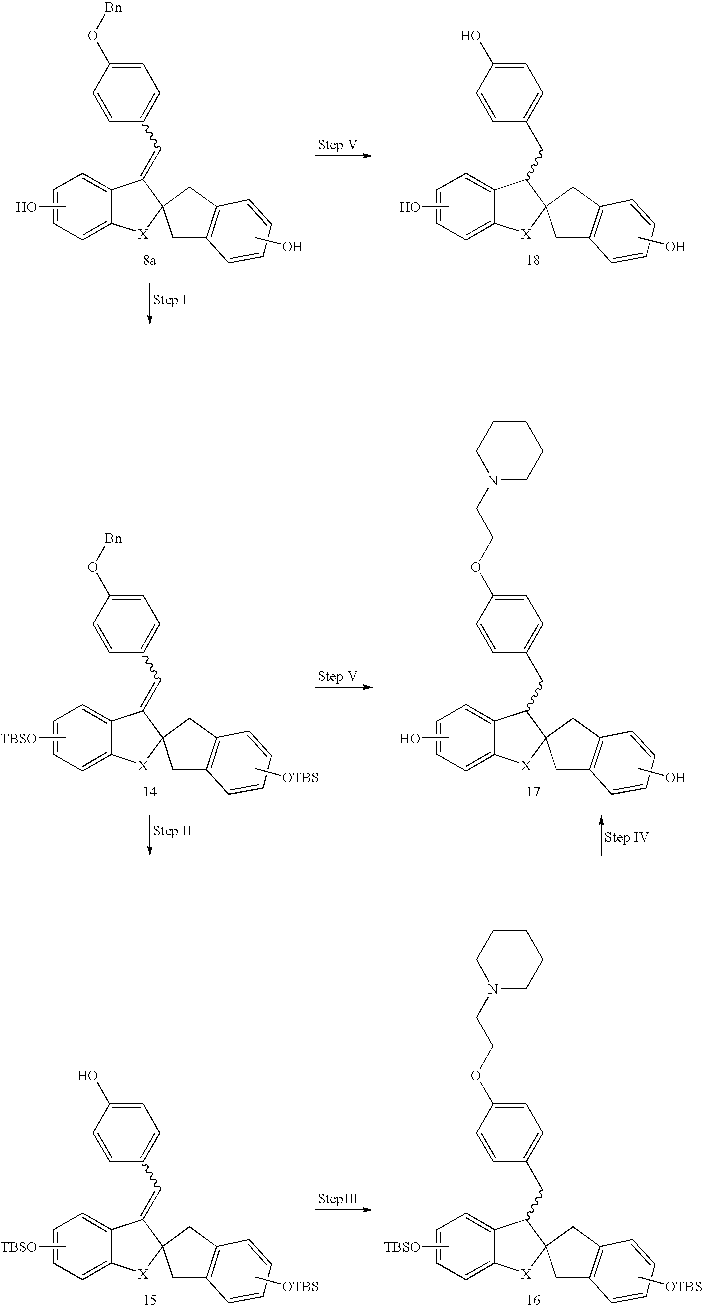

Representative protocols for step I (method J), step II and V (method K), step III (method L) as well as step IV (method M) in Scheme 3 are as follows:

Method J:

A mixture of the dihydroxy substrate (1.0 eq.), tert-butyldimethylchlorosilane (2.2 eq.) and imidazole (4.0 eq.) in DMF was stirred at the given temperature for the time indicated. After addition of EtOAc, the organic phase was washed with water, dried (anhydrous magnesium sulfate) and concentrated to give the crude product.

Method K:

A flask containing a solution of the substrate (1.0 eq.) and catalytic amount of 10% palladium on carbon in ethanol or methanol, was evacuated and filled with hydrogen three times before stirring the mixture at room temperature and atmospheric pressure for the time indicated. Workup was done by filtering the mixture through a short plug of celite®, followed by concentration of the filtrate in vacuo to obtain the crude product

Method L:

To a cool (dry-ice/CCl₄ bath) and stirred solution of the phenolic substrate (1.0 eq.), triphenylphosphine (8.2 eq.) and N-(2-hydroxyethyl)piperidin (8.2 eq.) in DCM was added a solution of diethyl azodicarboxylate (8.0 eq.) in DCM. The mixture was then allowed to stand at 0-4° C. overnight before quenching with saturated Ammonium chloride (aq.). The organic materials were then taken up in diethyl ether. This solution was dried (anhydrous magnesium sulfate), filtered and concentrated in vacuo to finish the crude product

Method M:

To a stirred solution of the silyl ether (1 eq.) in THF, was added a solution of tetrabutylammonium fluoride (1 M in THF) at room temperature. The mixture was stirred at that temperature for 1 day before quenching with saturated Ammonium chloride (aq.). The organic material was taken up in EtOAc, dried (anhydrous magnesium sulfate), and concentrated in vacuo to give the crude product.

The following examples represent preferred, but non-limiting embodiments of the invention. Examples 1-8, 15-17, 21, 22, 33-37, 39-43, 47 and 48 are comparative examples and are outside the scope of the new claims.

### EXAMPLE 1

### 5′-Hydroxy-1,3,3′-trihydro-2,2-spirobi(2H-indene)-1′-one

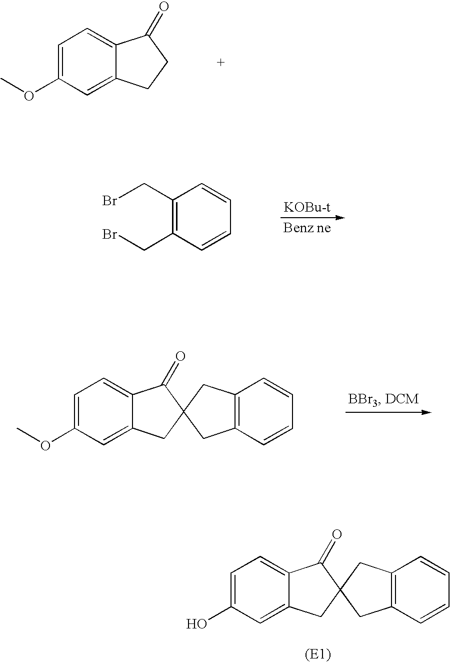

Step 1. A mixture of 5-methoxy-indanone-1 (3.24 g, 20 mmol), o-xylene dibromide (5.28 g, 20 mmol) and potassium t-butoxide (4.49 g, 40 mmol) in benzene was heated under reflux overnight. The reaction mixture was treated with 10% hydrochloric acid and the benzene phase was separated and washed with water and brine. The organic was dried, filtered and concentrated. The resulting residue was purified by chromatography on silica gel eluted with ethyl acetate/light petroleum ether (1/8). Pure fractions were pooled and concentrated affording 5′-methoxy-1,3,3′-trihydro-2,2′-spirobi(2H-indene)-1′-one which was purified by recrystallization from methanol to give white crystals. ¹H NMR (CDCl₃): ä 7.75 (d, 1H), 7.24-7.15 (m, 4H), 6.93 (dd, 1H), 6.85 (d, 1H), 3.88 (s, 3H), 3.49 (d, 2H), 3.12 (s, 2H), 2.81 (d, 2H). GC-MS: 264.19.

Step 2. To the mixture of above compound (264 mg, 1 mmol) in dichloromethane, was added 4 mL of boron tribromide (1M in CH₂Cl₂) at −78° C. The mixture was stirred at room temperature under nitrogen for 4 days and then was treated with ice-water. The organic phase was washed with brine, dried (magnesium sulfate), filtered and concentrated. The residue was purified by column chromatography on silica gel eluted with ethyl acetate/light petroleum ether (1/3). The combined pure fractions were concentrated, recrystallized from methanol and petroleum ether, to afford 5′-hydroxy-1,3,3′-trihydro-2,2′-spirobi(2H-indene)-1′one. ¹HNMR(acetone-D₆): ä 9.39 (s, OH), 7.61 (d, 1H), 7.25-7.14 (m, 4H), 6.96-6.89 (m, 2H), 3.34 (d, 2H), 3.09 (s, 2H), 2.84 (d, 2H). GC-MS: 249.99.

### EXAMPLE 2

### 5′-Hydroxy-1,1′,3,3′-tetrahydro-2,2′-spirobi(2H-indene)

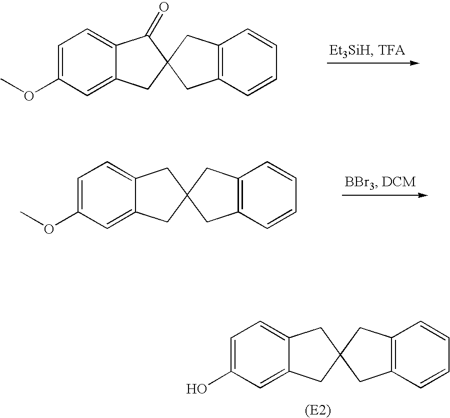

Step 1. A mixture of 5′-methoxy-1,3,3′-trihydro-2,2′-spirobi(2H-indene)-1′-one (528 mg, 2 mmol), triethylsilane (581 mg, 5 mmol) in 7 mL of trifluoro acetic acid was stirred at room temperature for 4 days. TFA was removed by evaporation under vacuum. The resulting oil was partitioned between ethyl acetate and saturated sodium bicarbonate solution and the organic phase was washed with brine, dried (anhydrous magnesium sulfate), filtered and concentrated. The residue was purified by column chromatography on silica gel eluted with benzene/heptane (3/7) affording a white solid. ¹H NMR (CDCl₃): 7.25-7.14 (m, 4H), ä 7.10 (d, 1H), 6.79 (d, 1H), 6.72 (dd, 1H), 3.81 (s, 3H), 2.99 (s, 4H), 2.96 (s, 2H), 2.92 (s, 2H). GC-MS: 250.02.

Step 2. A 325 mg portion of the above solid was dissolved in dichloromethane and treated with 4 mL of boron tribromide (1M in CH₂Cl₂) at −78° C. The mixture was stirred at room temperature under nitrogen for 10 hr and then was treated with ice-water. The organic phase was washed with brine, dried (magnesium sulfate), filtered and concentrated. The residue was chromatographed on silica gel eluted with 5% ethyl acetate in benzene. Pure fractions were pooled and concentrated, affording 5′-hydroxy-1,1′,3,3′-tetrahydro-2,2′-spirobi(2H-indene). ¹H NMR (CDCl₃): 7.26-7.15 (m, 4H), ä 7.05 (d, 1H), 6.71 (d, 1H), 6.65 (dd, 1H), 5.02 (s, OH), 2.99 (s, 4H), 2.94 (s, 2H), 2.91 (s, 2H). GC-MS: 236.14.

### EXAMPLE 3

### 5,5′-Dimethoxy-1,1′,3,3′-tetrahydro-2,2′-spirobi(2H-indene)-1-one

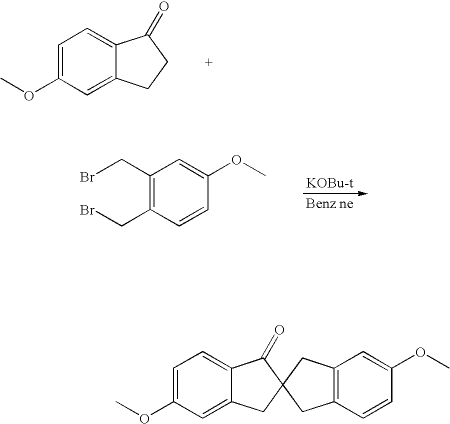

To a mixture of 5-methoxy-indanone-1 (4.95 g, 30.6 mmol) and 1,2-bis[bromomethyl]-4-methoxybenzene [J. Am. Chem. Soc. 116, 10593-60(1994); U.S. Pat. No. 4,210,749] (9 g, 30.6 mmol) in 200 mL of benzene, was added potassium t-butoxide (7.56 g, 67.3 mmol) in portions. The reaction mixture became warm and refluxed without heating. After 2 hr stirring at room temperature, the reaction mixture was partitioned between water and ethyl acetate. The organic phase was washed with 10% hydrochloric acid and brine, dried (anhydrous magnesium sulfate), filtered and concentrated. The resulting residue was purified by gradient chromatography on silica gel eluted with ethyl acetate/light petroleum ether from 1:8 to 1:4. Pure fractions were pooled and concentrated affording 5,5′ dimethoxy-1,1′,3,3′-tetrahydro-2,2′-spirobi(2H-indene)-1-one. ¹H NMR (CDCl₃): 7.73 (d, 2H), ä 7.09 (d, 1H), 6.92 (dd, 1H), 6.87-6.70 (m, 3H), 3.87 (s, 3H), 3.78 (s, 3H), 3.43 (t, 2H), 3.11 (s, 2H), 2.74 (dd, 2H). GC-MS: 294.0.

### EXAMPLE 4

### 5,5′-Dihydroxy-1,1′, 3,3′-tetrahydro-2,2-spirobi(2H-indene)-1-one

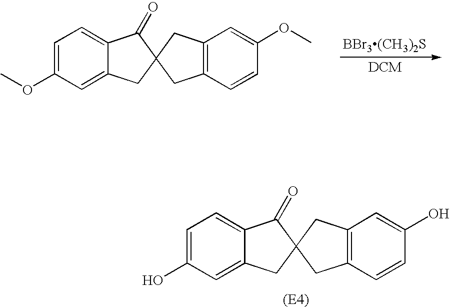

To a mixture of 5,5′-dimethoxy-1,1′,3,3′-tetrahydro-2,2′-spirobi(2H-indene)-1-one (883 mg, 3 mmol) in dichloromethane, was added 5 mL of boron trifluoride-methyl sulfide complex at −78° C. The mixture was stirred at room temperature for 2 days and then was poured into a beaker with ice-water and large volume of ethyl acetate. The organic phase was washed with brine, dried (magnesium sulfate), filtered and concentrated. The residue was chromatographed on silica gel eluted with ethyl acetate/light petroleum ether (1/4). Pure fractions were pooled and concentrated affording 5,5′-dihydroxy-1,1′,3,3′-tetrahydro-2,2′-spirobi(2H-indene)-1-one. ¹HNMR (CD₃OD): ä 7.60 (d, 1H), 6.97 (d, 1H), 6.87-6.78 (m, 2H), 6.66-6.57 (m, 2H), 3.25 (t, 2H), 3.03 (s, 2H), 2.69 (dd, 2H). GC-MS: 410.6 (TMSCl silylated).

### EXAMPLE 5

### 5,5-Dihydroxy-1,1′,3,3′-tetrahydro-2,2′-spirobi(2H-indene)

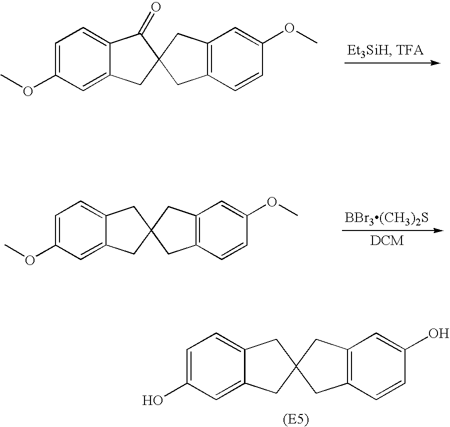

A mixture of 5,5′-dimethoxy-1,1′,3,3′-tetrahydro-2,2′-spirobi(2H-indene)-1-one (260 mg, 0.88 mmol), triethylsilane (257 mg, 2.21 mmol) in 5 mL of TFA was stirred at room temperature for 4 days. TFA was removed by evaporation under vacuum. The resulting oil was partitioned between chloroform and saturated aqueous sodium bicarbonate solution and the organic phase was washed with brine, dried (anhydrous magnesium sulfate), filtered and concentrated. The residue was purified by column chromatography on silica gel eluted with benzene/heptane (3/7) affording 380 mg of oil that contains triethylsilane. A solution of half of the above oily product in dichloromethane was treated with 4 mL of boron trifluoride-methyl sulfide complex at −78° C. The mixture was stirred at room temperature for 2 days and then was poured into a beaker with ice-water and large volume of ethyl acetate. The organic phase was washed with brine, dried (magnesium sulfate), filtered and concentrated. The residue was chromatographed on silica gel eluted with ethyl acetate/light petroleum ether (1/4). Pure fractions were pooled and concentrated affording 5,5′-dihydroxy-1,1′,3,3′-tetrahydro-2,2′-spirobi(2H-indene). ¹H NMR (acetone d₆): ä 8.01 (s, 2OH), 6.96 (d, 2H), 6.69 (d, 2H), 6.61 (q, 2H), 2.83 (s, 4H), 2.80 (s, 4H). GC-MS: 396.4 (TMSCl silylated).

### EXAMPLE 6

### 4-Bromo-5-methoxy-5′-hydroxy-1,1′, 3,3′-tetrahydro-2,2′-spirobi(2H-indene 1-one; and 4-bromo-5,5′-dihydroxy-1,1′,3,3′-tetrahydro-2,2′-spirobi(2H-indene)-1-one

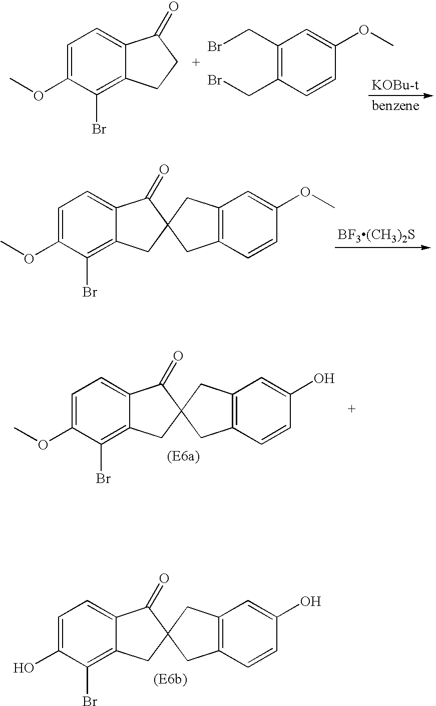

Step 1. To a mixture of 5-methoxy-4-bromo-indanone-1 (1.0 g, 4.1 mmol) and 1,2-bis[bromomethyl]4-methoxybenzene (1.22 g, 4.1 mmol) in 20 mL of benzene, was added potassium t-butoxide (988 mg, 8.8 mmol) in portions. The reaction mixture was heated under reflux overnight and then was partitioned between water and ethyl acetate. The organic phase was washed with 10% hydrochloric acid and brine, dried (anhydrous magnesium sulfate), filtered and concentrated. The residue was purified by column chromatography on silica gel eluted with ethyl acetate/light petroleum ether (1/8). Pure fractions were pooled and concentrated affording 4-bromo-5,5′-dimethoxy-1,1′,3,3′-tetrahydro-2,2′-spirobi(2H-indene)-1-one. ¹H NMR (CDCl₃): δ 7.75 (d, 1H), 7.10 (d, 1H), 6.95 (d, 1H), 6.76 (d, 1H), 6.73 (dd, 1H), 3.98 (s, 3H), 3.78 (s, 3H), 3.42 (t, 2H), 3.08 (s, 2H), 2.77 (dd, 2H).

Step 2. To the mixture of above compound (700 mg, 1.88 mmol) in 50 mL of dichloromethane, was added 9 mL of boron trifluoride-methyl sulfide complex at 0° C. The mixture was stirred at room temperature for 4 days and then was treated with ice-water and ethyl acetate. The organic phase was washed with brine, dried (magnesium sulfate), filtered and concentrated. The residue was purified by gradient chromatography on silica gel eluted with ethyl acetate/light petroleum ether (from 1:4 to 1:1). Pure fractions were pooled and concentrated, affording the mono-demethylated 4-bromo-5-methoxy-5′-hydroxy-1,1′,3,3′-tetrahydro-2,2′-spirobi(2H-indene)-1-one and 4-bromo-5,5′-dihydroxy-1,1′,3,3′-tetrahydro-2,2′-spirobi(2H-indene)-1-one. 4-bromo-5-methoxy-5′-hydroxy-1,1′,3,3′-tetrahydro-2,2′-spirobi(2H-indene)-1-one: ¹H NMR (acetone-D₆): ä 8.14 (s, OH), 7.72 (d, 1H), 7.22 (d, 1H), 7.02 (d, 1H), 6.73 (d, 1H), 6.66 (dd, 1H), 4.04 (s, 3H), 3.27 (t, 2H), 3.08 (s, 2H), 2.82 (dd, 2H). 4-bromo-5,5′-dihydroxy-1,1′,3,3′-tetrahydro-2,2′-spirobi(2H-indene)-1-one: ¹H NMR (acetone-D₆): ä 9.97 (s, OH), 8.14 (s, OH), 7.58 (d, 1H), 7.10 (d, 1H), 7.03 (d, 1H), 6.73 (d, 1H), 6.67 (dd, 1H), 3.27 (t, 2H), 3.06 (s, 2H), 2.81 (dd, 2H). GC-MS: 489.17, 491.17(TMSCl silylated).

### EXAMPLE 7

### 4-Bromo-5,5′-dihydroxy-1,1′,3,3′-tetrahydro-2,2′-spirobi(2H-indene)

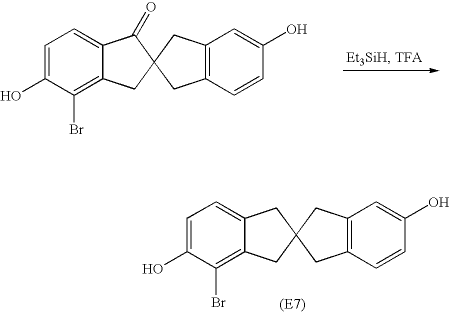

A mixture of 4-bromo-5,5′-dihydroxy-1,1′,3,3′-tetrahydro-2,2′-spirobi(2H-indene)-1-one (200 mg, 0.57 mmol), triethylsilane (1 g, 8.6 mmol) in 6 mL of TFA was stirred at room temperature for 2 days. TFA was removed by evaporation under vacuum. The resulting oil was partitioned between ethyl acetate and saturated sodium bicarbonate solution and the organic phase was washed with brine, dried (anhydrous magnesium sulfate), filtered and concentrated. The residue was purified by column chromatography on silica gel eluted with ethyl acetate/light petroleum ether (1/4). Pure fractions were pooled and concentrated, affording 4-bromo-5,5′-dihydroxy-1,1′,3,3′-tetrahydro-2,2′-spirobi(2H-indene). ¹H NMR (acetone-D₆): ä 8.57 (s, OH), 8.09 (s, OH), 6.98 (d, 1H), 7.10 (dd, 2H), 6.78 (d, 1H), 6.69 (d, 1H), 6.61 (dd, 1H), 2.94 (d, 4H), 2.86 (d, 4H). GC-MS: 474.2 (TMSCl silylated).

### EXAMPLE 8

### 5,5′-Dihydroxy-4-methyl-1,1′,3,3′-tetrahydro-2,2′-spirobi(2H-indene)-1-one

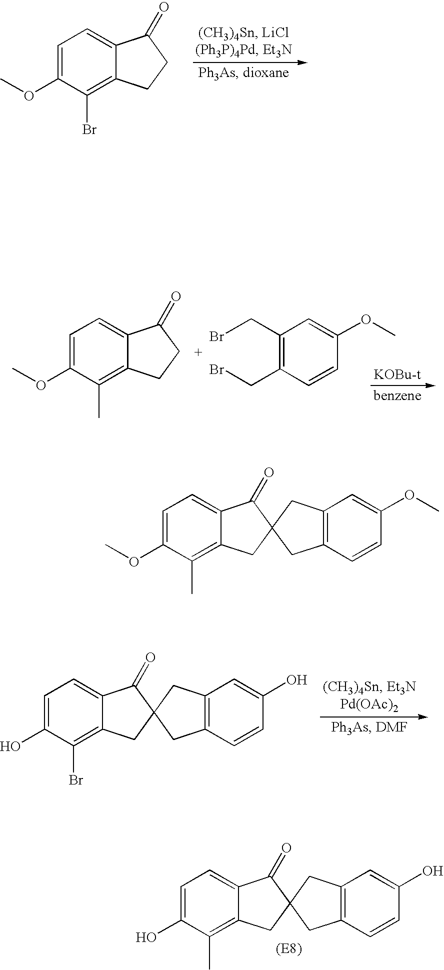

Step 1. A mixture of 5-methoxy-4-bromo-indanone-1 (241 mg, 1 mmol), tetrakis(triphenylphosphine)palladium(0) (35 mg, 0.03 mmol), tetramethyltin (215 mg, 1.2 mmol) in 6 mL of 1,3-dioxane was stirred in a sealed tube at 98° C. overnight. The reaction was not complete. Triphenylarsine (8 mg, 0.03 mmol), LiCl (124 mg, 3 mmol), triethyl amine (303 mg, 3 mmol) and 2 mL of DMF were added into the reaction mixture and the mixture was stirred at 120° C. overnight. The catalyst was removed by filtration (celite) and the filtrate was concentrated. The residue was purified by column chromatography on silica gel eluted with ethyl acetate/light petroleum ether (1/4). Pure fractions were pooled and concentrated affording 5-methoxy-4-methyl-indanone-1. ¹H NMR (CDCl₃): ä 7.59 (d, 1H), 6.85 (d, 1H), 3.88 (s, 3H), 2.98-2.92 (m, 2H, 2.66-2.60 (m, 2H), 2.14 (s, 3H). GC-MS: 176.3.

Step 2. A mixture of 5-methoxy-4-methyl-indanone-1 (138 mg, 0.78 mmol), 1,2-bis[bromomethyl]-4-methoxybenzene (230 mg, 0.78 mmol) and potassium t-butoxide (192 mg, 1.72 mmol) in 20 mL of benzene was heated at 104° C. overnight and then was partitioned between 10% HCl and ethyl acetate. The organic phase was washed with brine, dried (anhydrous magnesium sulfate), filtered and concentrated. The residue was purified by column chromatography on silica gel eluted with 5% ethyl acetate in benzene. Pure fractions were pooled and concentrated affording 5,5′-dimethoxy-4-methyl-1,1′,3,3′-tetrahydro-2,2′-spirobi(2H-indene)-1-one. ¹H NMR (CDCl₃): ä 7.69 (d, 1H), 7.10 (d, 1H), 6.92 (d, 1H), 6.77 (s, 1H), 6.73 (dd, 1H), 3.92 (s, 3H), 3.79 (s, 3H), 3.44 (t, 2H), 3.04 (s, 2H), 2.74 (dd, 2H), 2.11 (s, 3H).

Step 3. A mixture of 4-bromo-5,5′-dihydroxy-1,1′,3,3′-tetrahydro-2,2′-spirobi(2H-indene-1-one (300 mg, 0.87 mmol), palladium acetate (5.85 mg, 0.026 mmol), Triphenylarsine (32 mg, 0.104 mmol), tetramethyltin (467 mg, 2.61 mmol) and 0.5 mL of triethyl amine in 10 mL of 1 DMF was sired in a sealed tube at 100° C. overnight. The catalyst was removed by filtration (celite) and the filtrate was concentrated. The residue was purified by column chromatography on silica gel eluted with ethyl acetate/light petroleum ether (1/4). Pure fractions were pooled and concentrated affording 5,5′-dihydroxy-4-methyl-1,1′,3,3′-tetrahydro-2,2′-spirobi(2H-indene)-1-one. ¹HNMR (CDCl₃): ä 7.43 (d, 1H), 7.02 (d, 1H), 6.95 (d, 1H), 6.74-6.62 (m, 2H), 3.24 (t, 2H), 3.04 (s, 2H), 2.74 (dd, 2H), 2.14 (s, 3H). LC-MS-Q+1: 281.0.

### EXAMPLE 9

### Anti-5,5′-dihydroxy-1,1′,3,3′-tetrahydro-2,2′-spirobi(2H-indene)-1-one-(N-methyl oxime); and Syn-5,5′-dihydroxy-1,1′,3,3′-tetrahydro-2,2′-spirobi(2H-indene)-1-one-(N-methyl oxime)

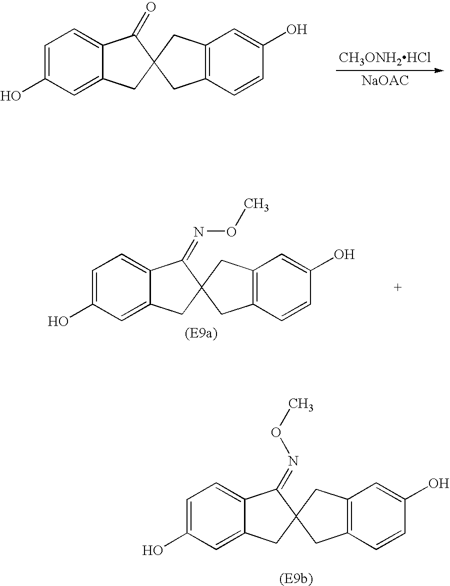

A mixture of methoxyamine hydrochloride (418 mg, 5 mmol) and sodium acetate (410 mg, 5 mmol) was dissolved in 5 mL of methanol and filtered after 5 min. The resulting solution was added to a mixture 5,5′-dihydroxy-1,1′,3,3′-tetrahydro-2,2′-spirobi(2H-indene)-1-one (133 mg, 0.5 mmol) and 500 mg of molecular sieves (4 Å) in 7 mL of methanol. The reaction mixture was stirred at 80° C. for 4 hr and then was concentrated in vacuum to remove methanol. The residue was partitioned between water and ethyl acetate. The organic phase was dried, filtered and concentrated. The residue was passed through a short silica gel column eluted with ethyl acetate/light petroleum ether (1/1). Pure fractions were pooled and concentrated to give the product. The anti-isomer was purified by recrystallization from methanol and the syn-isomer was isolated from mother liquor.

Anti-5,5′dihydroxy-1,1′,3,3′-tetrahydro-2,2′-spirobi(2H-indene)-1-one-(N-methyl oxime). ¹H NMR (DMSO-D₆): ä 8.01 (d, 1H), 6.97 (d, 1H), 6.76-6.52 (m, 4H), 3.82 (s, 3H), 3.18 (t, 2H), 2.90 (s, 2H), 2.84-2.72 (m, 2H). GC-MS:295.1.

Syn-5,5′-dihydroxy-1,1′,3,3′-tetrahydro-2,2′-spirobi(2H-indene)-1-one-(N-methyl oxime). ¹H NMR (CD₃OD): ä 7.47 (d, 1H), 6.99-6.94 (m, 1H), 6.73-6.56 (m, 4H), 3.81 (s, 3H), 3.27-3.22 (m, 2H), 2.96 (s, 2H), 2.71-2.61 (m, 2H). GC-MS: 295.1.

### EXAMPLE 10

### 5,5′-Dihydroxy-1,1′,3,3′-tetrahydro-2,2′-spirobi(H-indene)-1-one-oxime; and 5′-hydroxy-5-methoxy-1,1′,3,3′-tetrahydro-2,2′-spirobi(2H-indene)-1-one-oxime

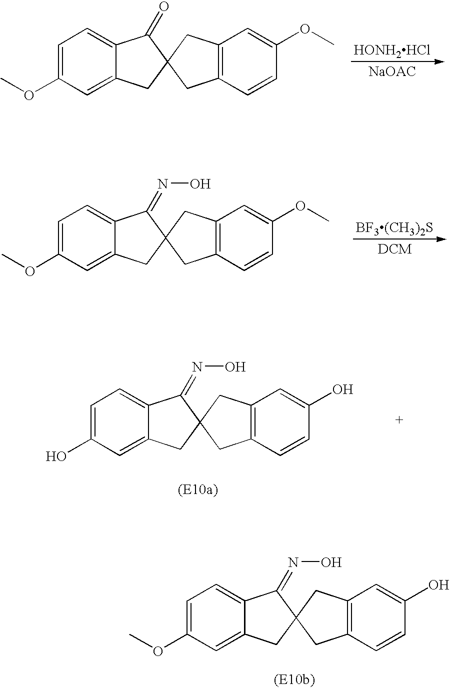

Step 1. A mixture of hydroxyamine hydrochloride (695 mg, 10 mmol) and sodium acetate (820 mg, 10 mmol) was dissolved in 20 mL of methanol and filtered after 2 min. The resulting clear solution was added to a mixture of 5,5′-dimethoxy-1,1′,3,3′-tetrahydro-2,2′-spirobi(2H-indene)-1-one (294 mg, 1 mmol) and 1 g of molecular sieves (4 Å) in 10 mL of methanol. The reaction mixture was heated in a sealed tube at 75° C. for 2 days and then was concentrated in vacuum to remove methanol. The residue was partitioned between water and ethyl acetate. The organic phase was washed with brine, dried (anhydrous magnesium sulfate), filtered and concentrated. The residue was purified on silica gel column eluted with ethyl acetate/toluene (5/95). Pure fractions were pooled and concentrated to give 5,5′-dimethoxy-1,1′,3,3′-tetrahydro-2,2′-spirobi(2H-indene)-1-one-oxime. ¹HNMR (acetone-D₆): ä 9.91 (s, 1H), 8.36 (d, 1H), 7.23-7.04 (m, 2H), 6.88-6.67 (m, 3H), 3.83 (s, 3H), 3.75 (s, 3H), 3.38-3.23 (m, 2H), 3.02 (s, 2H), 2.91-2.78 (m, 2H).

Step 2. To the mixture of above compound (18 mg, 0.09 mmol) in 3 mL of dichloromethane was added 1.2 mL of boron trifluoride-methyl sulfide complex at 0° C. The mixture was stirred at room temperature for 7 hr and then was treated with ice-water and ethyl acetate. The organic phase was washed with brine, dried (magnesium sulfate), filtered and concentrated. The residue was purified by chromatography on silica gel eluted with ethyl acetate/light petroleum ether (1:1). Pure fractions were pooled and concentrated, affording the mono-demethylated 5′-hydroxy-5-methoxy-1,1′,3,3′-tetrahydro-2,2′-spirobi(2H-indene)-1-one-oxime and 5,5′-dihydroxy-1,1′,3,3′-tetrahydro-2,2′-spirobi(2H-indene)-1-one-oxime. 5′-hydroxy-5-methoxy-1,1′,3,3′-tetrahydro-2,2′-spirobi(2H-indene)-1-one-oxime. ¹H NMR (CD₃OD): ä 8.35 (d, 1H), 6.97 (d, 1H), 6.86-6.81 (m, 2H), 6.63-6.54 (m, 2H), 3.81 (s, 3H), 3.38-3.23 (m, 2H), 3.01 (s, 2H), 2.82-2.73 (m, 2H). 5,5′-dihydroxy-1,1′,3,3′-tetrahydro-2,2′-spirobi(2H-indene)-1-one-oxime. ¹H NMR (CD₃OD): ä 8.27-8.22 (m, 1H), 6.96 (d, 1H), 6.70-6.54 (m, 4H), 3.30 (t, 2H), 2.94 (s, 2H), 2.83-2.73 (m, 2H).

### EXAMPLE 11

### 5,5′-Dihydroxy-1,1′,3,3′-tetrahydro-2,2′-spirobi(2H-indene)-1-methylidene

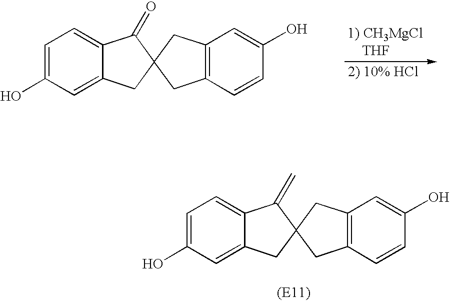

To a solution of 5,5′-dihydroxy-1,1′,3,3′-tetrahydro-2,2′-spirobi(2H-indene)-1-one (100 mg, 9 mmol) in 10 mL of anhydrous THF, was added a solution of methyl magnesium chloride (3.0 M in THF, 3 mL) at −70° C. The reaction mixture was stirred at room temperature overnight and then treated with 10% HCl. The mixture was extracted with ethyl acetate and the organic phase was washed with brine, dried (anhydrous magnesium sulfate) filtered and concentrated. The residue was purified by chromatography on silica gel eluted with ethyl acetate/light petroleum ether (3/7). Pure fractions were pooled and concentrated to give 5,5′-dihydroxy-1,1′,3,3′-tetrahydro-2,2′-spirobi(2H-indene)-1-methylidene. ¹H NMR (acetone-D₆): ä 8.45 (s, OH), 8.08 (s, OH), 7.38 (d, 1H), 6.98 (d, 1H), 6.76-6.60 (×4H), 5.24 (s, 1H), 4.80 (s, 1H), 3.12-2.99 (m, 2H), 2.90 (s, 2H), 2.89-2.80 (m, 2H). GC-MS: 264.3.

### EXAMPLE 12

### 5,5′-Dihydroxy-1-methyl-1,1′,3,3′-tetrahydro-2,2′-spirobi(2H-indene)

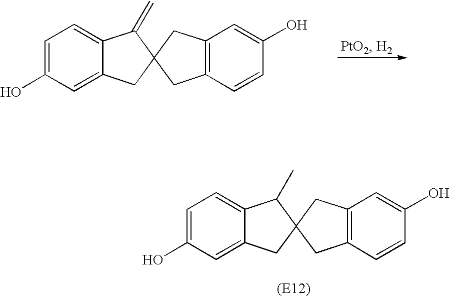

A mixture of 5,5′-dihydroxy-1,1′,3,3′-tetrahydro-2,2′-spirobi(2H-indene)-1-methylidene (35 mg, 0.13 mmol) and PtO₂ (20 mg) in 7 mL of ethyl acetate was hydrogenated under atmospheric pressure with stirring overnight. The catalyst was removed by filtration (celite) and the filtrate was concentrated. The residue was purified by column chromatography on silica gel eluted with ethyl acetate/light petroleum ether (1/4). Pure fractions were pooled and concentrated. The resulting oily product was crystallized from ether and petroleum ether to give 5,5′-dihydroxy-1-methyl-1,1′,3,3′-tetrahydro-2,2′-spirobi(2H-indene). ¹H. NMR (acetone-D₆): ä 8.01 (s, OH), 7.99 (s, OH), 7.02-6.92 (d, 1H), 6.75-6.53 (m, 4H), 3.01-2.42 (m, 7H), 1.11 (d, 3H). GC-MS: 266.6.

### EXAMPLE 13

### 1-Butyl-5,5′-hydroxy-1,1′,3,3′-tetrahydro-2,2′-spirobi(2H-indene)

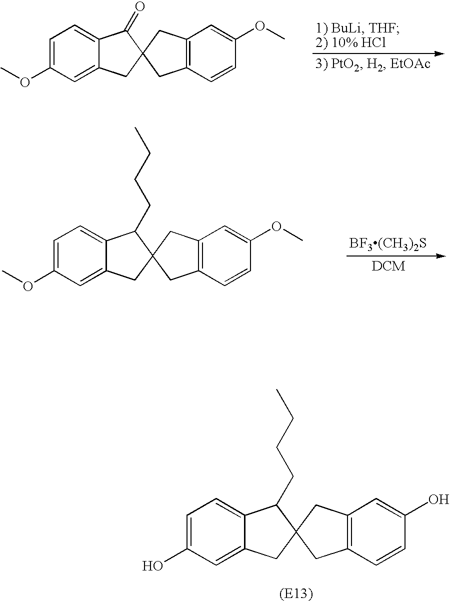

Step 1. To a solution of 5,5′-Dimethoxy-1,1′,3,3′-tetrahydro-2,2′-spirobi(2H-indene)-1-one (66 mg, 0.22 mmol) in 10 mL of THF, was added 0.55 mL of n-butyllithium (1.6M in hexane) at −70° C. The reaction mixture was stirred at room temperature overnight and then treated with 10% HCl. After 0.5 hr stirring, the reaction mixture was partitioned between water and ethyl acetate. The organic phase was washed with brine, dried (anhydrous magnesium sulfate), filtered and concentrated. A mixture of the resulting oil and PtO₂ (10 mg) in 10 mL of ethyl acetate was stirred under hydrogen from a balloon for 3 days. The catalyst was removed by filtration (celite) and the residue was purified by chromatography on silica gel eluted with ethyl acetate/light petroleum ether (1/8). Pure fractions were pooled and concentrated to give 1-butyl-5,5′-dimethoxy-1,1′,3,3′-tetrahydro-2,2′-spirobi(2H-indene). ¹H NMR (CDCl₃): ä 7.13-7.01 (m, 2H), 6.79-6.64 (m, 4H), 3.78 (s, 3H), 3.77 (s, 3H), 3.07 (dd, 1H), 2.94-2.63 (m, 6H), 1.69-1.24 (m, 6H), 0.86 (t, 3H). GC-MS: 336.3.

Step 2. A mixture of the above compound (40 mg, 0.12 mmol) and 2 mL of boron trifluoride-methyl sulfide complex in 5 mL of dichloromethane was stirred at room temperature overnight. The mixture was partitioned with ice-water and ethyl acetate. The organic phase was washed with brine, dried (anhydrous magnesium sulfate), filtered and concentrated. The residue was purified by chromatography on silica gel eluted with ethyl acetate/light petroleum ether (from 1:4 to 1:2). Pure fractions were pooled and concentrated affording 24 mg (66%) of 1-butyl-5,5′-hydroxy-1,1′,3,3′-tetrahydro-2,2′-spirobi(2H-indene). ¹H NMR (acetone-D₆): ä 9.39 (s, OH), 8.00 (s, OH), 7.23-6.90 (m, 2H), 6.79-6.54 (m, 4H), 3.05-2.51 (m, 7H), 1.71-1.20 (m, 6H), 0.87 (t, 3H). GC-MS: 452.4 (TMSCl silylated).

### EXAMPLE 14

### 5-Hydroxy-5′-(2″-piperidinylethoxy)-1-(p-methoxy)benzylildene-1,1′,3,3′-tetrahydro-2,2′-spirobi(H-indene); 6-hydroxy-5′-(2′-piperidinylethoxy)-1-(p-methoxy)benzylidene-1,1′,3,3′-tetrahydro-2,2′-spirobi(2H-indene); Z-5-hydroxy-5′-(2″-piperidinylethoxy)-1-(m-methoxy)benzylidene-1,1′,3,3′-tetrahydro-2,2′-spirobi(2H-indene); and Z-5-hydroxy-5′-(2″-piperidinylethoxy)-1-(m-hydroxy)benzylidene-1,1′,3,3′-tetrahydro-2,2′-spirobi(2H-indene)

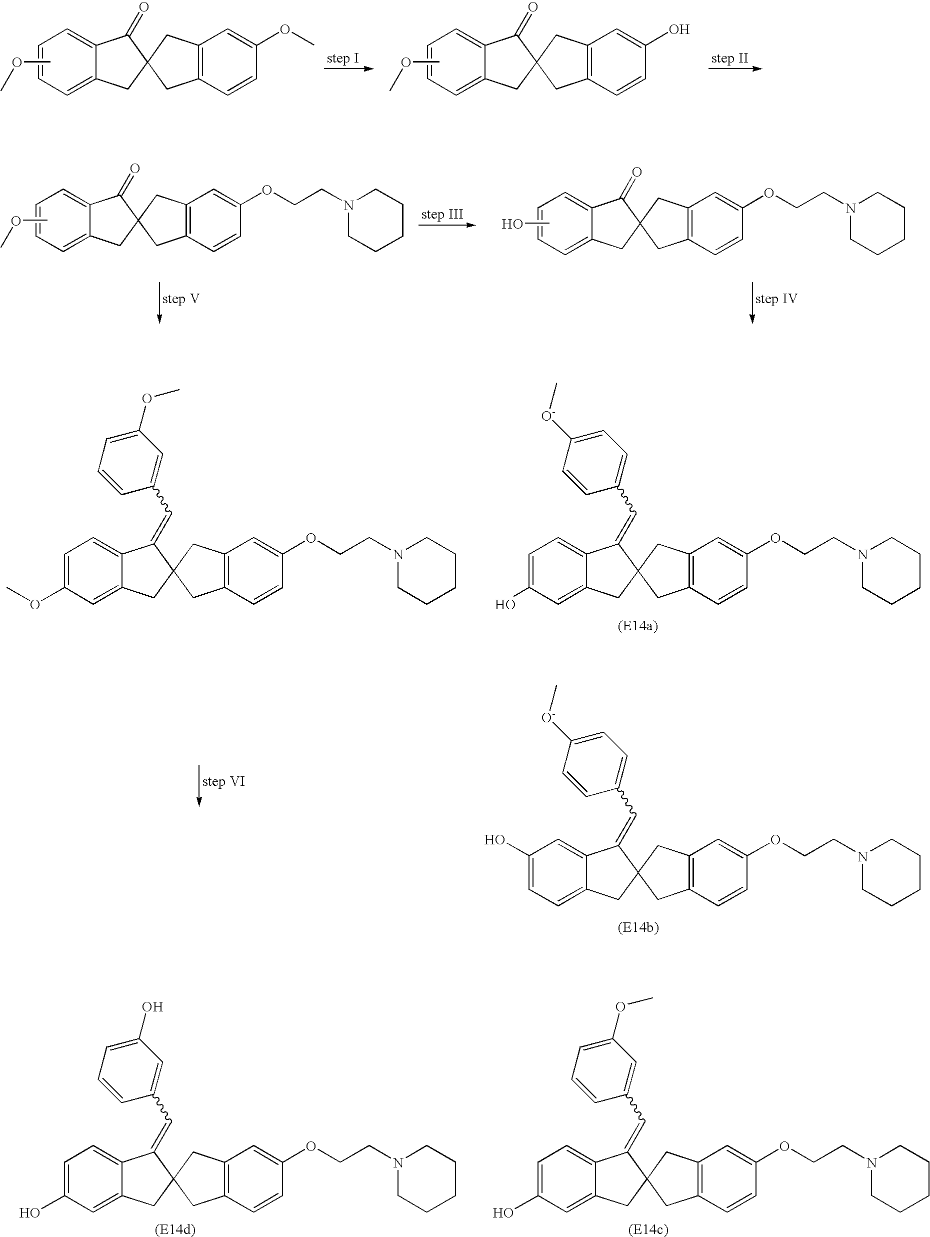

Step 1. 5-Methoxy-5′-hydroxy-1,1′,3,3′-tetrahydro-2,2′-spirobi(2H-indene)-1-one. To a solution of 5,5′-dimethoxy-1,1′,3,3′-tetrahydro-2,2′-spirobi(2H-indene)-1-one (1.03 g, 3.5 mmol) in 50 mL of dichloromethane was added dropwise 3.5 mL of boron tribromide (1 M in CH₂Cl₂) at −78° C. under nitrogen. The mixture was allowed to stand at −23° C. for 2 days. The reaction mixture was partitioned between water and EtOAc and the organic phase was then washed with brine, dried (anhydrous magnesium sulfate), filtered and concentrated. The residue was purified by column chromatography on silica gel eluted with ethyl acetate/petroleum ether (1:4) to yield 5-methoxy-5′-hydroxy-1,1′,3,3′-tetrahydro-2,2′-spirobi(2H-indene)-1-one. ¹H NMR (acetone-D₆): ä 8.10 (s, OH), 7.63 (d, 1H), 7.06-6.95 (m, 3H), 6.75-6.64 (m, 2H), 3.94 (s, 3H), 3.26 (t, 2M), 3.14 (s, 2H), 2.75 (dd, 2H). LC-MS-Q+1: 280.6, LC-MS-Q−1: 279.1. 6-Methoxy-5′-hydroxy-1,1′,3,3′-tetrahydro-2,2′-spirobi(2H-indene)-1-one (60%). ¹H NMR(acetone-D₆): ä 8.12 (s, OH), 7.41 (d, 1H), 7.23 (d, 1H), 7.12 (dd, 1H), 6.95 (d, 1H), 6.70-6.66 (m, 2H), 3.80 (s, 3H), 3.22 (t, 2H), 3.06 (s, 2H), 2.75 (dd, 2H). LC-MS-Q+1: 280.6, LC-MS-Q−1: 279.1.

Step 2. 5-Methoxy-5′-(2″-piperidinylethoxy)-1,1′,3,3′-tetrahydro-2,2′-spirobi(2H-indene)-1-one. A mixture of 5-methoxy-5′-hydroxy-1,1′,3,3′-tetrahydro-2,2′-spirobi(2H-indene)-1-one (600 mg, 2.14 mmol), N-(2-chloroethyl)-piperidine hydrochloride (1.58 g, 8.56 mmol) and K₂CO₃ (1.181 g, 8.56 mmol) in 100 mL of CH₃CN was stirred under reflux for 24 hr. After cooling to room temperature, K₂CO₃ was removed by filtration and rinsed with large quantity of EtOAc. The organic phase was washed with water, dried, filtered and concentrated. The residue was purified by column chromatography on silica gel eluted with diethyl ether plus 2% triethylamine. Pure fractions were pooled and concentrated in vacuum to give 5-methoxy-5′-(2″-piperidinylethoxy)-1,1′,3,3′-tetrahydro-2,2′-spirobi(2H-indene)-1-one. ¹H NMR (oxalate in CD₃OD): ä 7.67 (d, 1H), 7.12 (d, 1H), 7.04-6.96 (m, 2H), 6.91-6.78 (m, 2H), 4.34 (t, 2H), 3.89 (s, 3H), 3.51 (t, 2H), 3.37-3.13 (m, 6H), 2.98-2.74 (m, 4H), 1.95-1.82 (m, 4H), 1.74-1.59 (m, 2H). LC-MS-Q+1: 392.2. 6-Methoxy-5′-(2″-piperidinylethoxy)-1,1′,3,3′-tetrahydro-2,2′-spirobi(2H-indene)-1-one. ¹H NMR (CDCl₃): ä 7.27-6.94 (m, 4H), 6.70-6.54 (m, 2H), 4.224.09 (m, 2H), 3.80 (s, 3H), 3.30 (dd, 2H), 3.01 (s, 2H), 2.87 (t, 2H), 2.76-2.50 (m, 6H), 1.71-1.59 (m, 4H), 1.52-1.40 (m, 2H). LC-MS-Q+1: 392.2.

Step 3. 5-Hydroxy-5′-(2″-piperidinylethoxy)-1,1′,3,3′-tetrahydro-2,2′-spirobi(2H-indene)-1-one. To a solution of 5-methoxy-5′-(2″-piperidinylethoxy-1,1′,3,3′-tetrahydro-2,2′-spirobi(2H-indene)-1-one (530 mg, 1.35 mmol) in 50 mL of CH₂Cl₂, was added 0.5 mL of BF₃.S(CH₃) at room temperature under nitrogen. The reaction mixture was stirred at room temperature for 3 days, followed by TLC. 10% NaHCO₃ water solution was added and then extracted with EtOAc (3×50 mL). The organic phase was washed with brine, dried (MgSO₄), filtered and concentrated. The residue was purified by column chromatography on silica gel eluted with EtOAc/MeOH/Et₃N (90:10:1). Pure fractions were pooled and concentrated to give 5-hydroxy-5′-(2″-piperidinylethoxy)-1,1′,3,3′-tetrahydro-2,2′-spirobi(2H-indene)-1-one. ¹H NMR (CDCl₃): ä 8.15 (s, OH), 7.28-6.97 (m, 4H), 6.75-6.53 (m, 2H), 4.20-4.05 (m, 2H), 3.31 (m, 2H), 3.01 (s, 2H), 2.82 (m, 2H), 2.74-2.54 (m, 6H), 1.74-1.59 (m, 4H), 1.52-1.40 (m, 2H). LC-MS-Q+1: 378.1, LC-MS-Q−1: 376.3. 6-Hydroxy-5′-(2″-piperidinylethoxy)-1,1′,3,3′-tetrahydro-2,2′-spirobi(2H-indene)-1-one (91%). ¹H NMR (CDCl₃): ä 8.88 (s, OH), 7.28-6.97 (m, 4H, 6.68-6.53 (m, 2H), 4.12-4.05 (m, 2H), 3.31 (dd, 2H), 3.01 (s, 2H), 2.82 (t, 2H), 2.74-2.54 (m, 6H), 1.74-1.59 (m, 4H), 1.52-1.40 (m, 2H). LC-MS-Q+1: 378.4, LC-MS-Q−1: 376.3.

Step 4. 5-Hydroxy-5-(2″-piperidinylethoxy)-1-(p-methoxy)benzylidene-1,1′,3,3′-tetrahydro-2,2′-spirobi(2H-indene). Magnesium turning (64 mg, 0.265 mmol) was placed in a flame-dried flask and activated with a tiny crystal of iodine. 1 mL of dry THF was added and followed by slow addition of a solution of 4-methoxy benzyl chloride (413 mg, 2.65 mmol) in 4 mL of dry THF. After stirring for 2 hr, a solution of 5-hydroxy-5′-(2″-piperidinylethoxy)-1,1′,3,3′-tetrahydro-2,2′-spirobi(2H-indene)-1-one (100 mg, 0.265 mmol) in 5 mL of dry THF was added into the flask at −70° C. under nitrogen. The reaction mixture was stirred at room temperature overnight and then treated with 10% HCl. After stirring for 2 hr, the reaction mixture was first treated with sodium bicarbonate until pH=8 and then extracted with EtOAc. The organic phase was washed with brine, dried (Na₂SO₄), filtered and concentrated. The residue was purified by column chromatography on silica gel eluted first with EtOAc and then with MeOH/EtOAc (5:95) plus 2% triethyl amine. The resulting crude product was further purified by preparative HPLC (silica column, 2% Et₃N in EtOAc). Pure fractions were pooled and concentrated. The residue was dissolved in diethyl ether and then treated with a clear solution of oxalic acid in ether to afford

Z-5-hydroxy-5′-(2′-piperidinylethoxy)-1-(p-methoxy)benzylidene-1,1′,3,3′-tetrahydro-2,2′-spirobi(2H-indene) oxalate as white solid, ¹HNMR (free base in acetone-D₆): δ 7.22 (dd, 2H), 7.12-7.06 (m, 2H), 6.89 (dd, 2H), 6.83-6.68 (m, 3H), 6.49-6.44 (m, 1H), 6.35 (s, 1H), 4.06 (t, 2H), 3.80 (s, 3H), 3.25-3.12 (m, 2H), 2.98-2.86 (m, 4H), 2.68 (t, 2H), 2.51-2.42 (m, 4H), 1.58-1.48 (m, 4H), 1.46-1.36 (m, 2H). LC-MS-Q+1: 482.2, LC-MS-Q−1: 480.1. 6-Hydroxy-5′-(2″-piperidinylethoxy)1-(p-methoxy)benzylidene-1,1′,3,3′-tetrahydro-2,2′-spirobi(2H-indene).

Z-6-Hydroxy-5′-(2″-piperidinylethoxy)-1-(p-methoxy)benzylidene-1,1′,3,3′-tetrahydro-2,2′-spirobi(2H-indene) (16%). ¹H NMR (free base in CD₃C1): δ 7.15 (s, OH), 7.19 (d, 2H), 7.06-6.99 (m, 2H), 6.81 (d, 2H), 6.70-6.57 (m, 4H), 6.44 (s, 1H), 4.01 (t, 2H), 3.79 (s, 3H), 3.23-3.11 (m, 2H), 2.96-2.81 (n, 4H), 2.70 (t, 2H), 2.52-2.40 (m, 4H), 1.64-1.53 (m, 4H), 1.48-1.37 (m, 2H). LC-MS-Q+1: 482.2, LC-MS-Q−1: 480.1.

E-6-Hydroxy-5′-(2″-piperidinylethoxy)-1-(p-methoxy)benzylidene-1,1′,3,3′-tetrahydro-2,2′-spirobi(2H-indene) (8.5%). ¹H NMR (oxalate in CD₃OD): δ 7.14 (d, 2H), 7.06-6.89 (m, 4H), 6.74-6.59 (m, 5H), 4.06 (t, 2H), 3.74 (s, 3H), 3.52 (dd, 2H), 3.33-3.19 (m, 2H), 2.96-2.85 (m, 2H), 2.80-2.48 (m, 6H), 1.66-1.55 (m, 4H), 1.51-1.40 (m, 2H). LC-MS-Q+1: 482.2, LC-MS-Q−1: 480.4.

Z-5-Hydroxy-5′-(2″-piperidinylethoxy)-1-(m-methoxy)benzylidene-1,1′,3,3′-tetrahydro-2,2′-spirobi(2H-indene) (1.6%) ¹HNMR (oxalate in CD₃OD): δ 7.22-6.65 (m, 10H), 6.43 (s, 1H), 4.33-4.25 (m, 2H), 3.77 (s, 3H), 3.65-3.43 (m, 6H), 3.06 (m, 2H), 2.98-2.80 (m, 4H), 2.00-1.68 (m, 4H), 1.65-1.42 (m, 2H). LC-MS-Q+1: 482.5, LC-MS-Q−1: 480.1.

Step 5. Z-5-Methoxy-5′-(2″-piperidinylethoxy)-1-(m-methoxy)benzylidene-1,1′,3,3′-tetrahydro-2,2-spirobi(2H-indene). Magnesium turning (96 mg, 4 mmol) was placed in a flame-dried flask and activated with a tiny crystal of iodine. 2 mL of dry diethyl ether was added and followed by slow addition of a solution of 3-methoxy benzyl chloride (314 mg, 2 mmol) in 5 mL of dry diethyl ether. After stirring for 3 hr, a solution of 5-methoxy-5′-(2″-piperidinylethoxy)-1,1′,3,3′-tetrahydro-2,2′-spirobi(2H-indene)-1-one (200 mg, 1.01 mmol) in 20 mL of dry THF was added dropwise at 0° C. under nitrogen. The reaction mixture was stirred at room temperature overnight and then treated with 10% H₂SO₄), (aq.). After stirring for 2 hr, the reaction mixture was first treated with NaHCO₃ until pH=8 and then extracted with EtOAc. The organic phase was washed with brine, dried (MgSO₄), filtered and concentrated. The residue was purified by column chromatography on silica gel eluted first with EtOAc and then with EtOAc/MeOH/Et₃N (90:10:1). Pure fractions were pooled and concentrated. The resulting crude product was further purified by preparative HPLC to give Z-5-methoxy-5′-(2″-piperidinylethoxy)-1-(m-methoxy)benzylidene-1,1′,3,3′-tetrahydro-2,2′-spirobi(2H-indene). ¹H NMR (CDCl₃): δ 7.22-6.80 (m, 10H), 6.52 (s, 1H), 4.15 (m, 2H), 3.77 (s, 3H), 3.51 (s, 3H), 3.25 (m, 2H), 3.06 (m, 4H), 2.80 (m, 2H), 2.65 (m, 4H), 1.68 (m, 4H), 1.48 (m, 2H). LC-MS-Q+1: 496.6.

Step 6. Z-b 6-Hydroxy-5′-(2″-piperidinylethoxy)-1-(m-hydroxy)benzylidene-1,1′,3,3′-tetrahydro-2,2′-spirobi(2H-indene). To a solution of Z-5-methoxy-5′-(2′-piperidinylethoxy)-1-(m-methoxy)benzylidene-1,1′,3,3′-tetrahydro-2,2′-spirobi(2H-indene) (25 mg, 0.05 mmol) in 5 mL of CH₂Cl₂, was added 0.6 mL of BF₃.S(CH₃)₂ at 0° C. under nitrogen. The reaction mixture was stirred at room temperature for 3 days, monitored by TLC. 5% Na₂CO₃ (aq.) was added and then extracted with EtOAc (3×10 mL). The organic phase was washed with brine, dried (MgSO₄), filtered and concentrated. The residue was purified by column chromatography on silica gel eluted with EtOAc/MeOH/Et₃N (90:10:1). Pure fractions were pooled and concentrated to give 7 mg (30%) of Z-5-hydroxy-5′-(2″-piperidinylethoxy)-1-(m-hydroxy)benzylidene-1,1′,3,3′-tetrahydro-2,2-spirobi(2H-indene). ¹H NMR (CD₃OD): δ 7.25-6.40 (m, 11H), 4.45 (m, 2H), 3.55 (m, 4H), 3.17 (m, 2H), 3.05 (m, 2H), 2.65 (m, 1H), 2.25 (dd, 2H), 2.05 (m, 1H), 1.87 (m, 4H), 1.55 (m, 2H). LC-MS-Q+1: 468.4, LC-MS-Q−1: 466.3.

### EXAMPLE 15

### 5′-Hydroxy-5-methoxy-3-methyl-1,1′,3,3′-tetrahydro-2,2′-spirobi(2H-indene)-1-one

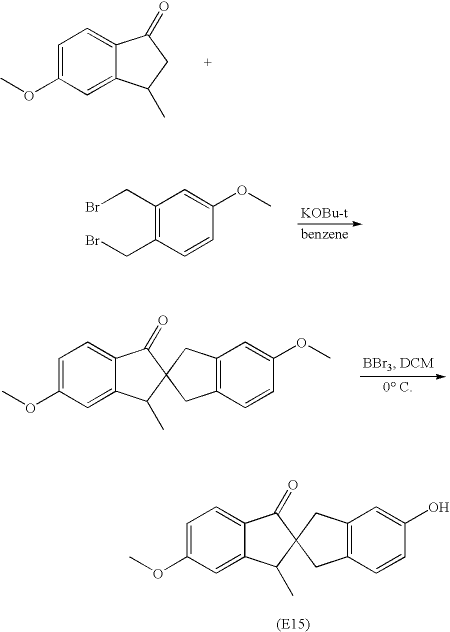

Step 1. To a mixture of 5-methoxy-3-methyl-indanone-1 [J. Pharm. Soc. Japan 74, 150-3(1954)] (528 mg, 3 mmol) and 1,2-bis[bromomethyl]-4-methoxybenzene (882 mg, 3 mmol) in 150 mL of benzene was added potassium t-butoxide (1.0 g, 9 mmol). The reaction mixture was stirred at room temperature for 30 minutes and then was heated to 40-45° C. for 4 hr. After cooling to room temperature, the reaction mixture was washed with 5×50 mL of water, dried over anhydrous magnesium sulfate, filtered and concentrated. The residue was purified by column chromatography on silica gel 60 eluted with heptane/ethyl acetate (8:3). Pure fractions were pooled and concentrated to yield 5,5′ dimethoxy-3-methyl-1,1′,3,3′-tetrahydro-2,2′-spirobi(2H-indene)-1-one. ¹H NMR (CDCl₃): δ 7.72 (1H, d), 7.06 (1H, m), 6.88 (2H, m), 6.72 (2H, m), 3.90 (3H, s), 3.77 (3H, m), 3.46 (1H, m), 3.20 (2H, m), 2.92 (1H, q), 2.75 (1H, q), 1.30 (3H, d). LC-MS-Q+1: 309.1.

Step 2. To a solution of 5,5′-dimethoxy-3-methyl-1,1′,3,3′-tetrahydro-2,2′-spirobi(2H-indene)-1-one (131 mg, 0.425 mmol) in 5 mL of CH₂Cl₂ at 0° C., was added 2 mL of boron tribromide (1M in CH₂Cl₂). The reaction mixture was stirred at 0° C. for 4 hr and then quenched with ethyl acetate. The organic layer was washed with 5×5 mL of brine, dried over anhydrous magnesium sulfate, filtered and concentrated. The residue was purified by flash chromatography (silica gel 60, heptane/ethyl acetate=6:4) to yield 5′-hydroxy-5-methoxy-3-methyl-1,1′,3,3′-tetrahydro-2,2′-spirobi(2H-indene)-1-one. ¹H NMR (CDCl₃): δ 7.73 (1H, d), 7.05 (1H, t) 6.92 (2H, m), 6.5 (2H, t), 3.88 (3×, s), 3.35 (1H, m), 3.22 (2H, m), 2.89 (1H, dd), 2.72 (1H, dd), 1.20 (3H, dd).

### EXAMPLE 16

### 5,5-Dihydroxy-3-methyl-1,1′,3,3′-tetrahydro-2,2′-spirobi(2H-indene)-1-one

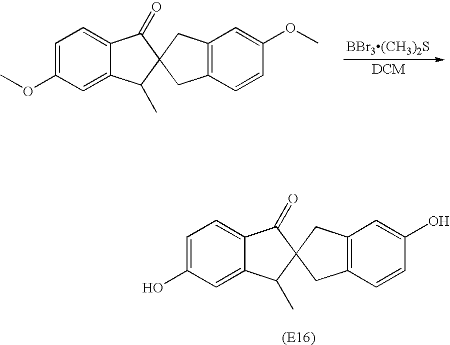

To a solution of 5,5′-dimethoxy-3-methyl-1,1′,3,3′-tetrahydro-2,2′-spirobi(2H-indene)-1-one (440 mg, 1.43 mmol) in 15 mL of dichloromethane was added 4 mL of boron trifluoride-methyl sulfide complex (38 mmol) at 0° C. The reaction mixture was stirred at 0° C. for 4 hr. After addition, the cooling bath was removed and the reaction mixture was stirred at room temperature for 72 hr. The reaction quenched by dropping 20 mL of ethyl acetate at 0° C. The organic layer was washed with 3×20 mL of brine, dried over anhydrous magnesium sulfate, filtered and concentrated. The residue was purified by flash chromatography (silica gel 60, heptane/ethyl acetate=1:1) to yield 5,5′-dihydroxy-3-methyl-1,1′,3,3′-tetrahydro-2,2′-spirobi(2H-indene)-1-one. ¹H NMR (acetone-D₆): δ 9.30 (1H, br s), 8.05 (1H, br s), 7.54 (1H, d), 7.00 (2H, m), 6.95 (1H, dd), 6.70 (1H, d), 6.60 (1H, m), 3.20 (2H, m), 3.10 (1H, m), 2.90 (1H, m), 2.75 (1H, m), 1.15 (3H, d). LC-MS-Q+1 280.9, LC-MS-Q−1: 279.1.

### EXAMPLE 17

### 1,5,5′-Trihydroxy-3-methyl-1,1′,3,3′-tetrahydro-2,2′-spirobi(2H-indene)

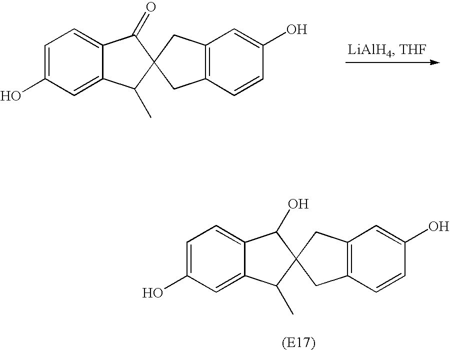

LiAlH₄ (72 mg, 2 mmol) was suspended in 5 mL of anhydrous THF at 0° C. 36 mg of 5,5′-dihydroxy-3-methyl-1,1′,3,3′-tetrahydro-2,2′-spirobi(2H-indene)-1-one (0.2 mmol) was dissolved in 10 mL of anhydrous THF and was added slowly into the flask. The reaction maintained at 0° C. for 1 hr and then at room temperature for another 1 hr. The reaction mixture was partitioned between aqueous saturated Ammonium chloride and 20 mL of ethyl acetate. The organic layer was washed with 3×20 mL of brine, dried over anhydrous magnesium sulfate, filtered and concentrated. The residue was purified by preparative TLC (silica gel GF, heptane/ethyl acetate=1:1) to yield 1,5,5′-trihydroxy-3-methyl-1,1′,3,3′-tetrahydro-2,2′-spirobi(2H-indene). ¹H NMR (acetone-D₆): δ 7.14 (1H, d), 6.94 (1H, m), 6.65 (4H, m), 4.70 (1H, m), 4.19 (1H, m), 3.15 (1H, m), 2.90 (2H, m), 2.55 (1H, m), 1.12 (3H, d). LC-MS-Q−1: 281.2.

### EXAMPLE 18

### 5,5′-Dihydroxy-1,3-dimethyl-1,1′,3,3′-tetrahydro-2,2′-spirobi(2H-indene)

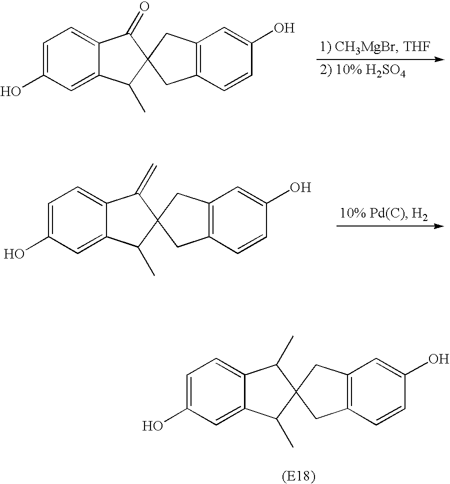

Step 1. 100 mg of 5,5′-dihydroxy-3-methyl-1,1′,3,3′-tetrahydro-2,2′-spirobi(2H-indene)-1-one (0.357 mmol) was dissolved in 5 mL of anhydrous THF and 5 mL of diethyl ether at 0° C., 2 mL of CH₃MgBr (20% in THF) was dropped into the above solution. After addition, ice bath was removed and the reaction mixture was stirred at room temperature overnight. The reaction quenched with 10% H₂SO₄ and followed by addition of ethyl acetate. The organic phase was washed with 3×15 mL of brine, dried over anhydrous magnesium sulfate, filtered and concentrated. The residue was purified by flash chromatography (silica gel 60, heptane/ethyl acetate=6:4) to yield 5,5′-dihydroxy-1-methylidene-3-methyl-1,1′,3,3′-tetrahydro-2,2′-spirobi(2H-indene). ¹H NMR (acetone-D₆): δ 8.33 (1H, br s), 7.99 (1H, br s), 7.35 (1H, d), 7.00 (1H, m), 6.67 (4H, m), 5.15 (1H, m), 4.67 (1H, m), 2.85 (5H, m), 1.12 (3H, d). LC-MS-Q+1: 279.1, LC-MS-Q−1: 277.0.

Step 2. A mixture of 5,5′-dihydroxy-1-methylidene-3-methyl-1,1′,3,3′-tetrahydro-2,2′-spirobi(2H-indene) (40 mg, 0.144 mmol) and 15 mg of Pd/C (10%) in 7 mL of ethanol was hydrogenated under atmospheric pressure at room temperature over night. The catalyst was removed by filtration (celite) and the filtrate was evaporated to dryness. The residue was purified by flash chromatography (silica gel 60, heptane/ethyl acetate=6:4) to yield 5,5′-dihydroxy-1,3-dimethyl-1,1′,3,3′-tetrahydro-2,2′-spirobi(2H-indene). ¹H NMR (acetone-D₆): δ 7.99 (1H, d), 6.98 (1H, m), 6.67 (4H, m), 2.97 (2H, d), 2.90 (2H, m), 2.52 (2H, d), 1.08 (3H, d), 1.02 (3H, d). LC-MS-Q−1: 279.1.

### EXAMPLE 19

### 5,5′-Dihydroxy-1-ethyl-3-methyl-1,1′,3,3′-tetrahydro-2,2′-spirobi(2H-indene)

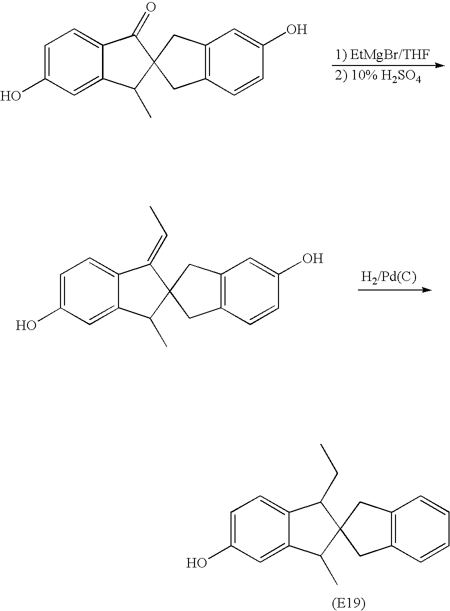

Step 1. 100 mg of 5,5′dihydroxy-3-methyl-1,1′,3,3′-tetrahydro-2,2′-spirobi(2H-indene)-1-one (0.357 mmol) was dissolved in 5 mL of anhydrous THF and 5 mL of diethyl ether at 0° C., 1 mL of CH₃CH₂MgBr (3M in THF) was dropped into the above solution. After addition, ice bath was removed and the reaction mixture was stirred at room temperature overnight. The reaction quenched with 10% H₂SO₄ and followed by addition of ethyl acetate. The organic phase was washed with 3×15 mL of brine, dried over anhydrous magnesium sulfate, filtered and concentrated. The residue was purified by flash chromatography (silica gel 60, heptane/ethyl acetate=6:4) to yield 5,5′-dihydroxy-1-ethylidene-3-methyl-1,1′,3,3′-tetrahydro-2,2′-spirobi(2H-indene). ¹H NMR (acetone-D₆): δ 8.31 (1H, br s), 7.95 (1H, br s), 7.38 (1H, d), 6.92 (11, m), 6.65 (4H, m), 5.22 (1H, q), 2.86 (5H, m), 1.85 (3H, d), 1.12 (3H, d). LC-MS-Q+1: 293.2, LC-MS-Q−1: 290.8.

Step 2. A mixture of 5,5′-dihydroxy-1-ethylidene-3-methyl-1,1′,3,3′-tetrahydro-2,2′-spirobi(2H-indene) (37 mg, 0.126 mmol) and 15 mg of Pd/C (10%) in 7 mL of ethanol was hydrogenated under atmospheric pressure at room temperature over night The catalyst was removed by filtration (celite) and the filtrate was evaporated to dryness. The residue was purified by flash chromatography (silica gel 60, heptane/ethyl acetate=6:4) to yield 17 mg (45.5%) of 5,5′-dihydroxy-1-ethyl-3-methyl-1,1′,3,3′-tetrahydro-2,2′-spirobi(2H-indene). ¹HNMR (acetone-D₆): δ 7.96 (1H, m), 6.98 (1H, m), 6.64 (4H, m), 2.68 (6H, m), 1.10 (8H, m). LC-MS-Q−1: 293.2.

### EXAMPLE 20

### 5,5′-Dihydroxy-1-propyl-3-methyl-1,1′,3,3′-tetrahydro-2,2′-spirobi(2H-indene)

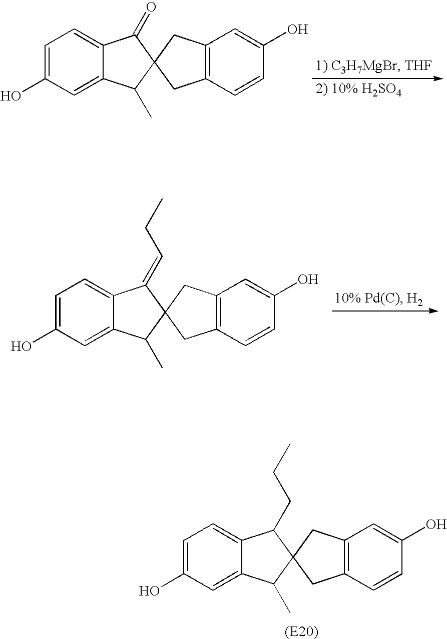

Step 1. 56 mg of 5,5′-dihydroxy-3-methyl-1,1′,3,3′-tetrahydro-2,2′-spirobi(2H-indene)-1-one (0.2 mmol) was dissolved in 5 mL of anhydrous THF and 1 mL of CF₃CH₂CH₂MgBr (2M in diethyl ether) was dropped into the above solution at 0° C. After stirring for 30 minutes, ice bath was removed and the reaction mixture was stirred at room temperature for 12 hr. The reaction mixture was treated with 10% H₂SO₄ and stirred for half-hr, followed by addition of ethyl acetate. The organic phase was washed with 3×10 mL of brine, dried over anhydrous magnesium sulfate, filtered and concentrated. The residue was purified by flash chromatography (silica gel 60, heptane/ethyl acetate=2:1) to yield 5,5′dihydroxy-1-propylidene-3-methyl-1,1′,3,3′-tetrahydro-2,2′-spirobi(2H-indene). ¹H NMR (CDCl₃): δ 7.37 (1H, d), 7.00 (1H, m), 6.65 (4H, m), 5.18 (1H, t), 5.10 (1H, br s), 4.75 (1H, br s), 2.95 (4H, m), 2.78 (1H, m), 2.35 (2H, m), 1.12 (3H, d), 1.00 (3H, t). LC-MS-Q+1: 307.3, LC-MS 41: 305.2.

Step 2. A mixture of 5,5′-dihydroxy-1-propylidene-3-methyl-1,1′,3,3′-tetrahydro-2,2′-spirobi(2H-indene) (47 mg, 0.15 mmol) and 10 mg of Pd/C (10%) in 5 mL of ethanol was hydrogenated under atmospheric pressure at room temperature for 30 hr. The catalyst was removed by filtration (celite) and the filtrate was evaporated to dryness. The residue was purified by flash chromatography (silica gel 60, heptane/ethyl acetate=6:4) to yield 5,5′-dihydroxy-1-propyl-3-methyl-1,1′,3,3′-tetrahydro-2,2′-spirobi(2H-indene). ¹H NMR (CDCl₃): δ 7.02 (2H, m), 6.60 (4H, m), 4.82 (1H, br s), 4.77 (1H, br s), 2.85 (6H, m), 2.60. (2H, m), 1.54 (2H, m), 1.10 (3H, d), 0.90 (3H, t). LC-MS-Q+1: 309.4, LC-MS-Q−1: 307.3.

### EXAMPLE 21

### 6′-Hydroxy-1,3,3′-trihydro-2,2′-spirobi(2H-indene)-1′-one

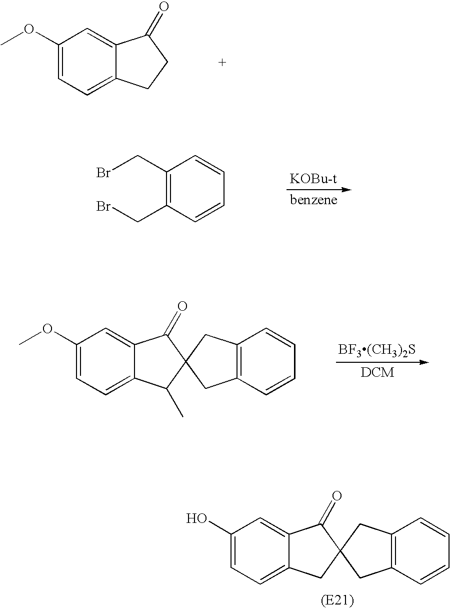

Step 1. A solution of o-xylene dibromide (10.56 g, 40 mmol) and 6-methoxy-1-indanone (3.24 g, 20 mmol) in 50 mL of benzene was added dropwise at room temperature to a suspension of potassium t-butoxide (6.75 g, 60 mmol) in benzene (50 mL). The mixture was stirred for 24 h at room temperature under nitrogen atmosphere. The reaction was monitored by TLC (5:95 EtOAc:benzene) until complete. The reaction mixture was treated with 10% HCl, washed with water, brine, and the organic phase was dried (anhydrous magnesium sulfate), filtered and concentrated. The residue was purified by column chromatography on silica gel eluted with ethyl acetate/petroleum ether (1:20) to yield 6′-methoxy-1,3,3′-trihydro-2,2′-spirobi(2H-indene)-1′-one.

Step 2. To a solution of 6′-methoxy-1,3,3′-trihydro-2,2′-spirobi(2H-indene)-1′-one (200 mg, 0.76 mmol) in 14 mL of dichloromethane was added 6.5 mL of boron trifluoride-methyl sulfide complex at 0° C. The mixture was stirred for 24 hr at room temperature under nitrogen. The reaction was monitored by TLC (1:8 EtOAc:p-ether) until complete. The reaction mixture was washed with water, brine, and the organic phase was dried (anhydrous magnesium sulfate), filtered and concentrated. The residue was purified by column chromatography on silica gel eluted with ethyl acetate/light petroleum ether (1:8) to yield 6′-hydroxy-1,3,3′-trihydro-2,2′-spirobi(2H-indene)-1′-one. ¹H NMR (acetone-D₆): ä 2.85 (d, 2H), 3.08 (s, 2H), 3.36 (d, 2H), 7.1-7.7.3 (m, 6H), 7.4 (d, 1H). GC-MS-Q: 250.2

### EXAMPLE 22

### 6,5′-Dihydroxy-1,1′,3,3′-tetrahydro-2,2′-spirobi(2H-indene)-1-one

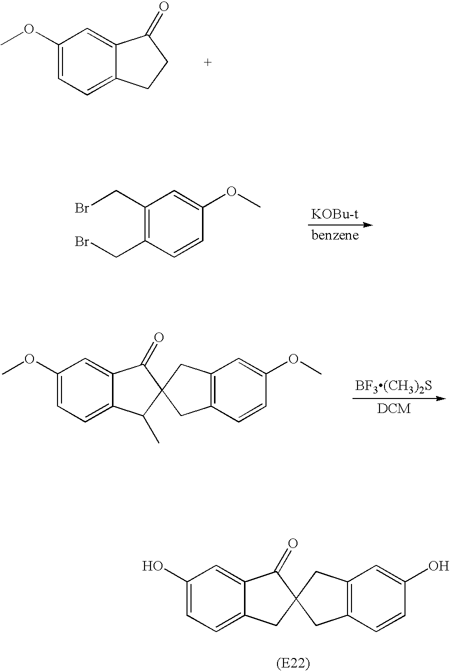

Step 1. A solution of 1,2-bisfbromomethyl]4-methoxybenzene (3.25 g, 1.1 mmol) and 6-methoxy-1-indanone (1.4 g, 8.5 mmol) in 50 mL of benzene was added dropwise at room temperature to a suspension of potassium t-butoxide (2.7 g, 24 mmol) in 50 mL of benzene. The mixture was stirred at 85° C. for 24 hr. The reaction was monitored by TLC (1:3 EtOAc: p-ether) until complete. The reaction mixture was treated with 10% HCl, washed with water, brine, and the organic phase was dried (anhydrous magnesium sulfate), filtered and concentrated. The residue was purified by column chromatography on silica gel eluted with ethyl acetate/light petroleum ether (1:8) to yield 6,5′-dimethoxy-1,1′,3,3′-tetrahydro-2,2′-spirobi(2H-indene)-1-one. ¹H NMR (CDCl₃): ä 7.36-7.17 (m, 3H), 27.09 (d, 1H), 6.79-6.67 (m, 2H), 3.84 (s, 3H), 3.78 (s, 3H), 3.42 (t, 2H), 3.09 (s, 2H), 2.77 (dd, 2H).

Step 2. To a solution of 6,5′-dimethoxy-1,1′,3,3′-tetrahydro-2,2-spirobi(2H-indene)-1-one (300 mg, 1.02 mmol) in 10 mL of dichloromethane, was added dropwise 6.5 mL of boron trifluoride-methyl sulfide complex. The mixture was stirred for 24 hr under nitrogen. The reaction was monitored by TLC (1:8 EtOAc: per) until complete. The reaction mixture was washed with water, brine, and the organic phase was dried (anhydrous magnesium sulfate, filtered and concentrated. The residue was purified by column chromatography on silica gel eluted with ethyl acetate/petroleum ether (1:3) to yield 6,5′-dihydroxy,1′,3,3′-tetrahydro-2,2′-spirobi(2H-indene)-1-one. ¹H NMR (acetone-D₆): ä 7.3 (d, 1H), 7.2 (dd, 1H), 7.1 (d, 1H), 7.0 (d, 1H), 6.7 (d, 1H), 6.6 (dd, 1H), 3.1-3.3 (m, 2H), 3.06 (s, 2H), 2.76 (dd, 1H). LC-MS-Q: 265.0.

### EXAMPLE 23

### 6,5′-Dihydroxy-1-methyl-1,1′,3,3-tetrahydro-2,2′-spirobi(2H-indene)

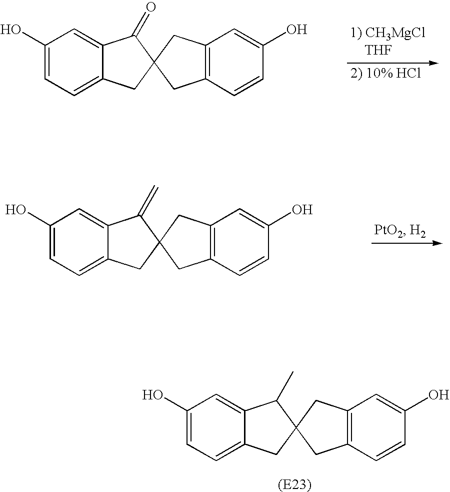

Step 1. To a solution of 6,5′-dihydroxy-1,1′,3,3′-tetrahydro-2,2′-spirobi(2H-indene)-1-one (100 mg, 0.38 mmol) in 10 mL of anhydrous THF was added a solution of methyl magnesium chloride (3.0 M in THF, 3 mL) at −70° C. The reaction mixture was stirred at room temperature overnight and then treated with 10% HCl. The mixture was extracted with ethyl at and the organic phase was washed with brine, dried (anhydrous magnesium sulfate) filtered and concentrated. The residue was purified by gradient chromatography on silica gel eluted with ethyl acetate/light petroleum ether (3/7). Pure fractions were pooled and concentrated to 6,5′-dihydroxy-1,1′,3,3′-tetrahydro-2,2′-spirobi(2H-indene)-1-methylidene. ¹H NMR (acetone-D₆): ä 8.20 (s, 1H), 7.53 (s, 1H), 7.07-6.96 (m, 3H), 6.67 (dd, 1H), 6.70 (d, 1H), 6.64 (dd, 1H), 5.36 (s, 1H), 4.95 (s, 1H), 3.12-2.98 (m, 2H), 2.94-2.81 (m, 4H). GC-MS: 408.04 (TMSCl silylated).

Step 2. A mixture of 6,5′-dihydroxy-1,1′,3,3′-tetrahydro-2,2′-spirobi(2H-indene)-1-methylidene (42 mg, 0.16 mmol) and PtO₂ (10 mg) in 5 mL of ethyl acetate was stirred under hydrogen from balloon at room temperature overnight. The catalyst was removed by filtration (celite) and the filtrate was concentrated. The residue was purified by column chromatography on silica gel eluted with ethyl acetate/light petroleum ether (1/2). Pure fractions were pooled and concentrated to give 6,5′-dihydroxy-1-methyl-1,1′,3,3′-tetrahydro-2,2′-spirobi(2H-indene). ¹H NMR (acetone-D₆): ä 7.99 (s, 1H), 7.93 (s, 1H), 6.95 (dd, 2H), 6.67 (s, 2H), 6.59 (dd, 21), 2.96 (s, 2H), 3.00-2.66 (m, 4H), 2.53-2.44 (m, 1H), 1.13 (d, 3H). GC-MS: 410.15 (TMSCl silylated).

### EXAMPLE 24

### 6,5′-Dihydroxy-1-ethyl-1,1′3,3′-tetrahydro-2,2′-spirobi(2H-indene)

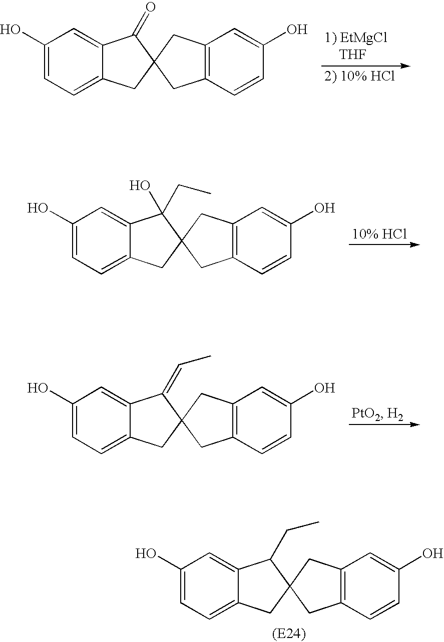

Step 1. To a solution of 6,5′ dihydroxy-1,1′,3,3′-tetrahydro-2,2′-spirobi(2H-indene)-1-one (200 mg, 0,76 mmol) in 10 mL of anhydrous THF, was added ethyl magnesium chloride (1 M in THF, 5 mL) at −70° C. The reaction mixture was stirred at room temperature overnight. The reaction was interrupted by addition of aqueous saturated ammonium chloride at 0° C. The reaction mixture was with ethyl acetate and the organic phase was washed with brine, dried (anhydrous magnesium sulfate) filtered and concentrated 1/3 of the residue was addressed to preparative HPLC separation (C8 column, ammonium acetate buffer/acetonitrile) to give 1,6,5′-trihydroxy-1-ethyl-1,1′,3,3′-tetrahydro-2,2′-spirobi(2H-indene) isomer-A and 1,6,5′-trihydroxy-1-ethyl-1,1′,3,3′-tetrahydro-2,2′-spirobi(2H-indene (isomer-B). Isomer-A: ¹H NMR (acetone-D₆): ä 6.96-6.85 (m, 2H), 6.80 (d, 1H), 6.72-6.61 (m, 2H), 6.60-6.53 (m, 1H), 3.34-3.10 (m, 2H), 2.72-2.60 (m, 3H), 2.18-2.09 (m, 1H), 1.85-1.64 (m, 2H), 0.95 (t, 3H). Isomer-B: ¹H NMR (acetone-D₆): ä 6.97 (dd, 2H), 6.81 (d, 1H), 6.65 (dd, 1H), 6.62-6.55 (m, 2H), 3.35 (d, 1H), 3.13 (d, 1H), 2.66 (d, 1H), 2.63 (s, 2H), 2.17 (d, 1H), 1.88-1.64 (m, 2H), 0.92 (t, 3H).

Step 2. A solution of racemic 1,6,5′-trihydroxy-1-ethyl-1,1,3,3′-tetrahydro-2,2′-spirobi(2H-indene) (60 mg, 0.2 mmol) in ethyl acetate was stirred with 10% HCl at room temperature for 0.5 hr. The mixture was extracted with ethyl acetate and the organic phase was washed with brine, dried (anhydrous magnesium site), filtered and concentrated. The residue was purified by column chromatography on silica gel eluted with ethyl acetate/light petroleum ether (1/2). Pure fractions were pooled and concentrated to give 6 5′-dihydroxy-1,1′,3,3-tetrahydro-2,2′-spirobi(2H-indene)-1-ethylidene. ¹H NMR (acetone-D₆): ä 8.13, 7.94 (s, 2OH), 7.14-6.83 (m, 3H), 6.73-6.54 (m, 3H), 6.09-5.47 (m, 1H), 3.00-2.74 (m, 6H), 1.95-1.70 (m, 3H).

Step 3. A mixture of 6 5′-dihydroxy-1,1′,3,3′-tetrahydro-2,2′-spirobi(2H-indene)-1-ethylene (30 mg, 0.11 mmol) and PtO₂ (10 mg) in 5 mL of ethyl acetate was sired under hydrogen from balloon at room temperature overnight. The catalyst was removed by filtration (celite) and the filtrate was concentrated. The residue was purified by gradient chromatography on silica gel eluted with ethyl acetate/light petroleum ether (from 1:3 to 1:1). Pure fractions were pooled and concentrated to give 6,5′-dihydroxy-1-ethyl-1 1′,3,3-tetrahydro-2,2-spirobi(2H-indene). ¹H NMR (CD₃OD): ä 6.98-6.85 (m, 21H), 6.68-6.64 (m, 1H), 6.58-6.49 (m, 3H), 3.04-2.93 (m, 1H), 2.81-2.54 (m, 6H), 1.77-1.63 (m, 1H), 1.49-1.37 (m, 1H), 2.17 (in, 1H), 0.99-0.91 (m, 3H).

### EXAMPLE 25

### 6,5′-Dihydroxy-1-butyl-1,1′,3,3′-tetrahydro-2,2′-spirobi(2H-indene)

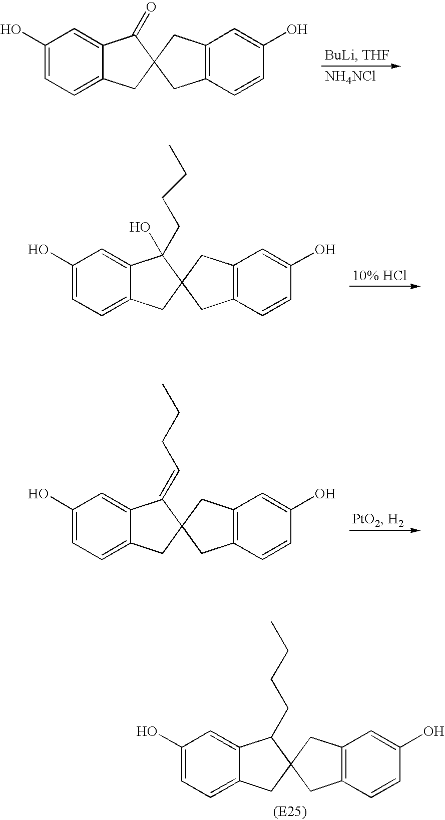

Step 1. To a solution of 6,5′-dihydroxy-1,1′,3,3′-tetrahydro-2,2′-spirobi(2H-indene)-1-one (160 mg, 0.6 mmol) in 15 mL of anhydrous THF, was added butyl lithium (2.5 M in heptane, 2 mL) at −70° C. The reaction mixture was stirred at room temperature overnight The reaction was interrupted by addition of aqueous saturated ammonium chloride. The reaction mixture was extracted with ethyl acetate and the organic phase was washed with brine, dried (anhydrous magnesium filtered and concentrated. Half of the residue was addressed to preparative HPLC separation (SiO₂, 2% ethyl acetate in heptane) to give 1,6,5′-trihydroxy-1-butyl-1,1′,3,3′-tetrahydro-2,2′-spirobi(2H-indene) isomer-A and 1,6,5′-trihydroxy-1-butyl-1,1′,3,3′-tetrahydro-2,2′-spirobi(2H-indene) isomer-B. Isomer-A: ¹H NMR (acetone-D₆): ä 8.07 (s, 1H), 7.94 (s, 1H), 7.07-6.91 (m, 2H). 6.81 (d, 1H), 6.70-6.52 (m, 3H), 3.83 (s, 1H), 3.36 (d, 1H), 3.12 (d, 1H), 2.71-2.62 (m, 3H), 2.15 (d, 1H, 1.76-1.54 (m, 2H), 1.37-1.15 (m, 4H), 0.89-0.80 (m, 3H). LC-MS-Q−1: 323.0. Isomer-B: ¹H NMR (acetone-D6): ä 8.08-7.84 (two broad peaks, 2H), 7.03-6.91 (, 2H), 6.82-6.78 (m, 1H), 6.67-6.54 (m, 3H), 3.86 (s, 1H), 3.34 (d, 1H), 3.13 (d, 1H), 2.71-2.60 (m, 3H), 2.15 (d, 1H), 1.77-1.54 (m, 2H, 1.38-1.14 (m, 4H), 0.90-0.80 (m, 3H). LC-MS-Q−1: 323.0.

Step 2. The other half of the above residue in ethyl acetate was stirred with 10% HCl at room temperature overnight. The mixture was extracted with ethyl acetate and the organic phase was washed with brine, dried (anhydrous magnesium sulfate), filtered and concentrated. The residue was purified by column chromatography on silica gel eluted with ethyl acetate/light petroleum ether (1/2). Pure fractions were pooled and concentrated to give 6,5′-dihydroxy-1,1′,3,3′-tetrahydro-2,2′-spirobi(2H-indene)-1-butylidene. (cis/trans=2:1) LC-MS-Q−1: 305.0. The corresponding cis- and trans-isomers were separated by preparative HPLC (C8 column, ammonium acetate buffer/acetonitrile=50°/) to give cis-6,5′-dihydroxy-1,1,3,3′-tetrahydro-2,2-spirobi(2H-indene)-1-butylidene and trans-6 5′-dihydroxy-1,1′,3,3′-tetrahydro-2,2′-spirobi(2H-indene)-1-butylidene. Cis-6,5′-dihydroxy-1,1′,3,3′-tetrahydro-2,2′-spirobi(2H-indene)-1-butylidene: ¹H NMR (CD₃OD): ä 7.09-6.89 (m, 3H), 6.66-6.53 (m, 3H), 5.42 (t, 1H), 3.05-2.90 (m, 2H), 2.85-2.73 (m, 4H), 2.44-2.31 (m, 2H), 1.53-1.40 (m, 2H), 0.95 (t, 3H). LC-MS-Q+1: 307.3, LC-MS-Q−1: 305.5. Trans-6,5′-dihydroxy-1,1′,3,3′-tetrahydro-2,2′-spirobi(2H-indene)-1-butylidene: ¹H NMR (CD₃OD): ä 6.97-6.91 (m, 2H), 6.81 (d, 1H), 6.69-6.57 (m, 3H), 5.91 (t, 1H), 3.51-3.37 (m, 2H, 2.97-2.78 (m, 4H), 2.20-2.08 (m, 2H), 1.52-1.34 (m, 2H), 0.91 (t, 3H). LC-MS-Q+1: 307.3, LC-MS-Q−1: 305.5.

Step 3. A mixture of 6,5′-dihydroxy-1,1′,3,3′-tetrahydro-2,2′-spirobi(2H-indene)-1-butylidene (45 mg, 0.14 mmol) and PtO₂ (10 mg) in 3 mL of ethyl acetate was stirred under hydrogen from balloon at room temperature for 1 day. The catalyst was removed by filtration (celite) and the filtrate was concentrated. The residue was purified by preparative HPLC separation (C8 column, ammonium acetate buffer/acetonitrile=50%) to give 11 mg (24%) of 6,5′-dihydroxy-1-butyl-1,1′,3,3′-tetrahydro-2,2′-spirobi(2H-indene) isomer-A and 13 mg (28%) of 6,5′-dihydroxy-1-butyl-1,1′,3,3′-tetrahydro-2,2′-spirobi(2H-indene) isomer-B. Isomer-A: ¹H NMR (CD₃OD): ä 6.91 (t, 2H), 6.66-6.61 (m, 2H), 6.57-6.50 (m, 2H), 3.00 (d, 1H), 2.82-2.56 (m, 6H), 1.67-1.24 (m, 6), 0.95-0.86 (m, 3H). LC-MS-Q+1: 309.3, LC-MS-Q−1: 307.6. Isomer-B: ¹H NMR (CD₃OD): A 6.94 (t, 2H), 6.65 (d, 1H), 6.58-6.51 (m, 3H), 2.96 (d, 1H), 2.81-2.55 (m, 6H), 1.65-1.24 (m, 6H), 0.94-0.86 (m, 3H). LC-MS-Q+1: 309.3, LC-MS-Q−1: 307.6.

### EXAMPLE 26

### 6,5′-Dihydroxy-1-benzylidene-1,1′,3,3′-tetrahydro-2,2-spirobi(2H-indene)

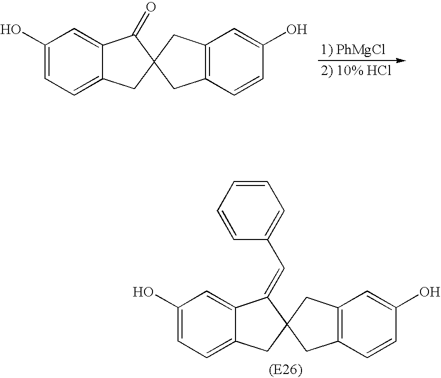

To a solution of 6,5′-dihydroxy-1,1′,3,3′-tetrahydro-2,2′-spirobi(2H-indene)-1-one (108 mg, 0.41 mmol) in 10 mL of anhydrous THF, was added a solution of benzyl magnesium chloride (1.0 M in THF, 2.4 mL) at −70° C. The reaction mixture was stirred at room temperature for 1 day and then treated with 10% HCl. After stirring 2 hr, mixture was extracted with ethyl acetate. The organic phase was washed with brine, dried (anhydrous magnesium sulfate), filtered and concentrated. The residue was purified by chromatography on silica gel eluted with TBME/heptane (1/3). Pure fractions were pooled and concentrated to give 6,5′-dihydroxy-1-benzylidene-1,1′,3,3′-tetrahydro-2,2′-spirobi(2H-indene). ¹H NMR (CD₃OD): ä 7.34-7.11 (m, 7H), 7.03-6.95 (m, 1H), 6.67-6.52 (m, 3H), 6.46 (s, 1H), 3.21-3.07 (m, 2H), 2.96-2.83 (m, 4H). LC-MS-Q−1: 339.4.

### EXAMPLE 27

### 6′,5′-Dihydroxy-1-(p-methoxy)benzylidene-1,1′,3,3′-tetrahydro-2,2′-spirobi(2H-indene)

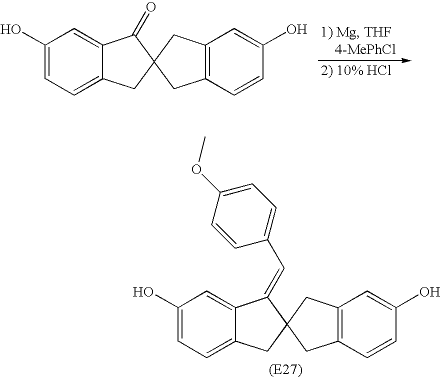

Magnesium turning (480 mg, 20 mmol) was placed in a flame-dried flask and activated with a tiny crystal of iodine. 5 mL of dry THF was added and followed by slow addition of a solution of 4-methoxy benzyl chloride (3.13 g, 20 mmol) in 15 mL of dry THF. After stirring for 3 hr, a solution of 6,5′-dihydroxy-1,1′,3,3′-tetrahydro-2,2′-spirobi(2H-indene)-1-one (134 mg, 0.5 mmol) in 10 mL of dry THF was added into the flask at 0° C. under nitrogen. The reaction mixture was stirred at room temperature overnight and then treated with 10% HCl. After refluxing for 1 hr, the mixture was extracted with ethyl acetate. The organic phase was washed with brine, dried (anhydrous magnesium sulfate) filtered and concentrated. The residue was purified by gradient chromatography on silica gel eluted with ethyl acetate/light petroleum ether from 1:4 to 1:2. Pure fractions were pooled and concentrated to give 6,5′-dihydroxy-1-(p-methoxy)benzylidene-1,1′,3,3′-tetrahydro-2,2′-spirobi(2H-indene). Preparative HPLC separation (C8 column, CH₃CN/NH₄OAc buffer, gradient) gave corresponding E- and Z-isomers. Z-6 5′-dihydroxy-1-(p-methoxy)benzylidene-1,1′,3,3′-tetrahydro-2,2′-spirobi(2H-indene). ¹H NMR (acetone-D₆): δ 8.09 (s, OH), 8.07 (s, OH), 7.23 (dd, 2H), 7.20-7.15 (m, 1H), 7.08-6.98 (m, 2H), 6.89 (dd, 2H), 6.82-6.63 (m, 3H), 6.50 (s, 1H), 3.81 (s, 3H), 3.22-3.08 (m, 2H), 2.97-2.67 (m, 4H). LC-MSQ+1: 371.2, LC-MS-Q−1: 369.1. E-6,5′ dihydroxy-1-(p-methoxy)benzylidene-1,1′,3,3′-tetrahydro-2,2′-spirobi(2H-indene). ¹HNMR (acetone-D₆): δ 8.21 (s, OH), 8.08 (s, OH), 7.20-7.14 (m, 3H), 7.08-6.96 (m, 4H), 6.78-6.64 (m, 4H), 3.74 (s, 3H), 3.56 (t, 2H), 2.95-2.68 (m, 4H). LC-MS-Q+1: 371.2, LC-MS 1: 369.1.

### EXAMPLE 28

### 6,5 ′-Dihydroxy-1-(p-hydroxy)-benzylidene-1,1′,3,3′-tetrahydro-2,2′-spirobi(2H-indene)

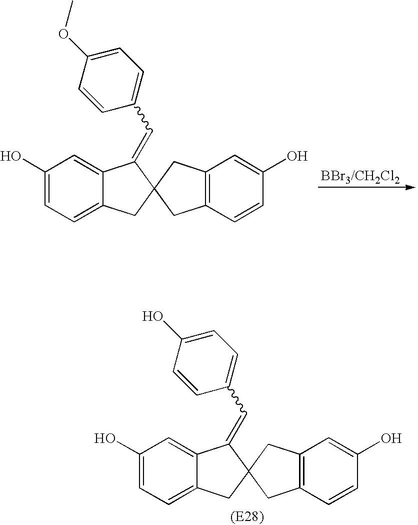

A solution of 6,5′-dihydroxy-1-(p-methoxy)benzylidene-1,1′, 3,3′-tetrahydro-2,2′-spirobi(2H-indene) (20 mg, 0.054 mmol) in 5 mL of CH₂Cl₂ was stirred under nitrogen at −70° C. Boron tribromide (1 mL, 1M in CH₂Cl₂) was added dropwise to the solution from a syringe and the reaction mixture was stand at −23° C. overnight. The reaction mixture was then partitioned between water and EtOAc (3×30 mL). The organic phase was dried, filtered and concentrated. The resulting residue was purified by preparative HPLC (C8-column, NH₄OAc buffer/CH₃CH=7:3) to give Z-6,5′-dihydroxy-1-(p-hydroxy)benzylidene-1,1′,3,3′-tetrahydro-2,2′-spirobi(2H-indene) and E-6,5′-dihydroxy-1-(hydroxy)benzylidene-1,1′,3,3′-tetrahydro-2,2′-spirobi(2H-indene). Z-6,5′-dihydroxy-1-(p-hydroxy)benzylidene-1,1′,3,3′-tetrahydro-2,2-spirobi(2H-indene). ¹H NMR (acetone-D₆): δ 8.45 (s, 1H), 8.31 (s, 1H), 8.23 (s, 1H), 7.18-7.06 (m, 3H), 6.91-6.82 (m, 4H), 6.78-6.71 (m, 4H), 3.18-2.88 (m, 5H), 2.61-2.51 (m, 1H). LC-MS Q+1: 357.1, LC-MS-Q−1: 355.0. E-6,5′ dihydroxy-1(p-dihydroxy)benzylidene-1,1′,3,3′-tetrahydro-2,2′-spirobi(2H-indene): ¹H NMR (acetone-D₆): δ 8.36 (s, 1H), 8.22 (s, 1H), 8.18 (s, 1H), 722-7.11 (m, 3H), 6.94-6.72 (m, 8H), 3.21-2.51 (m, 6H). LC-MS-Q+1: 357.1, LC-MS-Q−1: 355.3.

### EXAMPLE 29

### Rac-(1′R,2S/1′S,2R)-6,5′-dihydroxy-1-(p-methoxy)benzyl-1,1′,3,3′-tetrahydro-2,2′-spirobi(2H-indene); and rac-(1′R,2R/1′S,2S)-6,5′-dihydroxy-1-(p-methoxy)benzyl-1,1′,3,3′-tetrahydro-2,2′-spirobi(2H-indene)

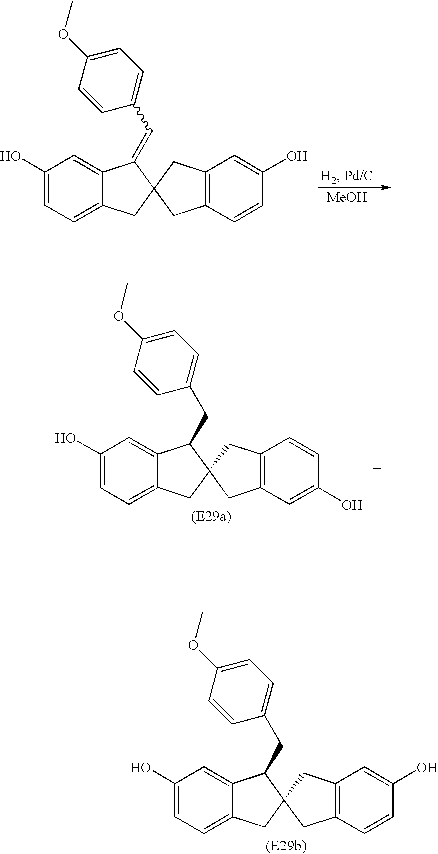

A solution of(Z/E)-6,5′-dihydroxy-1-(p-methoxy)benzylidene-1,1,3,3′-tetrahydro-2,2′-spirobi(2H-indene) (13 mg, 0.035 mmol) and 10% palladium on carbon (5 mg) in 10 mL of methanol was hydrogenated under atmospheric pressure with stirring at room temperature for 2 days. The catalyst was removed by filtration (celite) and the filtrate was concentrated. The resulting residue was purified by preparative HPLC (C8-column, CH₃CN/NH₄OAc buffer, gradient) to afford Rac-(1′R,2S/1′S,2R)-6,5′-dihydroxy-1-(p-methoxy)benzyl-1,1′,3,3′-tetrahydro-2,2′-spirobi(2H-indene) and Rac-(1′R,2R/1′S,2S-6,5′-dihydroxy-1-(methoxy)benzyl-1,1′,3,3′-tetrahydro-2,2′-spirobi(2H-indene). Rac-(1′R,2S/1′S,2R)-6,5′-dihydroxy-1-(p-methoxy)-benzyl-1,1′,3,3′-tetrahydro-2,2′-spirobi(2H-indene). ¹H NMR (acetone-D₆, 500 MHz): δ 8.11 (s, OH), 7.89 (s, OH), 7.04 (dd, 2H), 6.95-6.89 (m, 2H), 6.82 (dd, 2H), 6.70 (d, 1H), 6.60-6.54 (m, 2H), 6.17 (d, 1H), 3.81 (s, 3H), 3.24-3.20 (m, 1H), 3.14 (d, 1H), 3.05-3.00 (m, 1H), 2.83-2.76 (m, 2H), 2.70-2.51 (m, 4H). LC-MS-Q+1: 373.0, LC-MS-Q−1: 371.2. Rac-(1′R,2R/1′S,2S)-6,5′ dihydroxy-1-(p-methoxy)benzyl-1,1′,3,3′-tetrahydro-2,2′-spirobi(2H-indene). ¹H NMR (acetone-D₆): δ 8.06 (s, OH), 7.89 (s, OH), 7.11 (dd, 2H), 7.04-6.92 (m, 2H), 6.88 (dd, 2H), 6.84-6.77 (m, 1H), 6.61-6.54 (m, 2H), 6.15 (d, 1H), 3.79 (s, 3H), 3.24-3.19 (m, 1H), 3.10-2.99 (m, 2H), 2.84-2.78 (m, 2H), 2.69-2.57 (m, 4H). LC-MS-Q+1: 373.3, LC-MS-Q−1: 370.6.

### EXAMPLE 30

### 6,5′-Di[(t-butyldimethyl)silyloxy]-1-[4-benzyloxy(benzylidene)]-1,1′,3,3′-tetrahydro-2,2′-spirobi(2-indene)

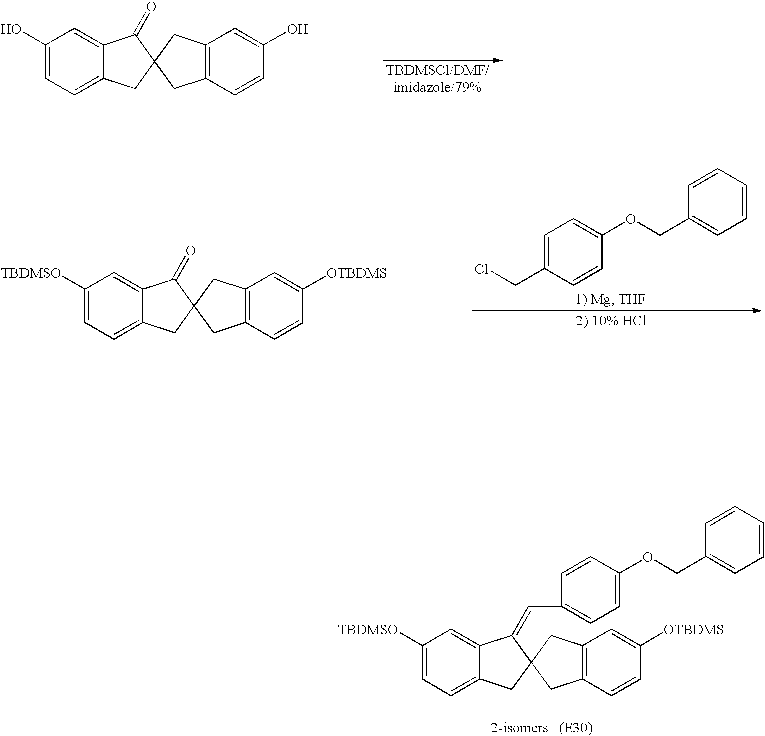

Step 1. A mixture of 6,5′-dihydroxy-1,1′,3,3′-tetrahydro-2,2′-spirobi(2H-indene)-1-one (700 mg, 2.61 mmol), t-butyldimethylsilyl chloride (866 mg, 5.75 mmol) and imidazole (711 mg, 10.4 mmol) in 10 mL of DMF was stirred under nitrogen at room temperature for 3 days. The reaction mixture was partitioned between ethyl acetate and water. The organic phase was washed with brine, dried (anhydrous magnesium sulfate), filtered and concentrated. The residue was purified by a short silica gel column, eluted with ethyl acetate/light petroleum ether (1:1). Pure fractions were pooled and concentrated, affording 6,5′-di[(t-butyldimethyl)silyloxy]-1,1′,3,3′-tetrahydro-2,2′-spirobi(2H-indene)-1-one. ¹H NMR (CDCl₃): δ 7.26-7.18 (m, 2H), 7.14-7.08 (m, 1H), 7.01 (d, 1H), 6.70-6.62 (m, 2H), 3.40 (t, 2H), 3.07 (s, 2H), 2.78-2.69 (m, 2H), 0.98 (dd, 18H), 0.20 (dd, 12H).

Step 2. 240 mg of magnesium (10 mmol) was placed in a flame-dried flask and activated with a tiny crystal of iodine. A solution of 4-benzyloxybenzyl chloride (2.33 g, 10 mmol) in 15 mL of anhydrous THF was added dropwise in 1 hr under nitrogen. After stirring for 1 hr, the flask was cooled to −78° C. and a solution of 6,5′-di[(t-butyldimethyl)silyloxy]-1,1′,3,3-tetrahydro-2,2′-spirobi(2H-indene)-1-one (495 mg, 1 mmol) in 10 mL of anhydrous THF was added into the flask. Cooling bath was removed and the reaction mixture was stirred at room temperature overnight. The reaction was interrupted by addition of 10 mL of 10% HCl and continued stirring for 2 hr. The reaction mixture was partitioned between ethyl acetate and water. The organic phase was washed with brine, dried (anhydrous magnesium sulfate), filtered and concentrated. The residue was purified by column chromatography on silica gel eluted with ethyl acetate/heptane (1:10). Pure fractions were pooled and concentrated to give a mixture of Z- and E-isomer, which was separated by HPLC (C8-column, CH₃CN) to afford Z-6,5′di[(t-butyldimethyl)silyloxy]-1-[4-benzyloxy(benzylidene)]-1,1′,3,3′-tetrahydro-2,2′-spirobi(2H-indene) and E-6,5-di[(t-butyldimethyl)silyloxy]-1-[4-benzyloxy(benzylidene)]-1,1′,3,3′-tetrahydro)-2,2′-spirobi(2H-indene). Z-6,5′-di[(t-butyldimethyl)silyl-oxy]-1-[4-benzyloxy(benzylidene)]-1,1′,3,3′-tetrahydro-2,2′-spirobi(2H-indene): ¹H NMR (CDCl₃: 500 MHz): δ 7.47-7.43 (m, 2H), 7.41-7.36 (in, 2H), 7.33-7.30 (m, 1H), 7.21 (d, 2H), 7.05 (dd, 2H), 6.89 (d, 2H), 6.70-6.68 (m, 1H), 6.66-6.62 (m, 3H), 6.41 (s, 1H), 5.06 (s, 2H), 3.23-3.15 (m, 2H), 2.97-2.86 (m, 4H), 0.99 (s, 9H, 0.90 (s, 9H), 0.22 (s, 6H), 0.02 (s, 6H). LC-MS-Q+1: 675.7.

### EXAMPLE 31

### 6,5-Dihydroxy-1-(p-benzyloxy)benzylidene-1,1′,3,3′-tetrahydro-2,2′-spirobi(2H-indene)

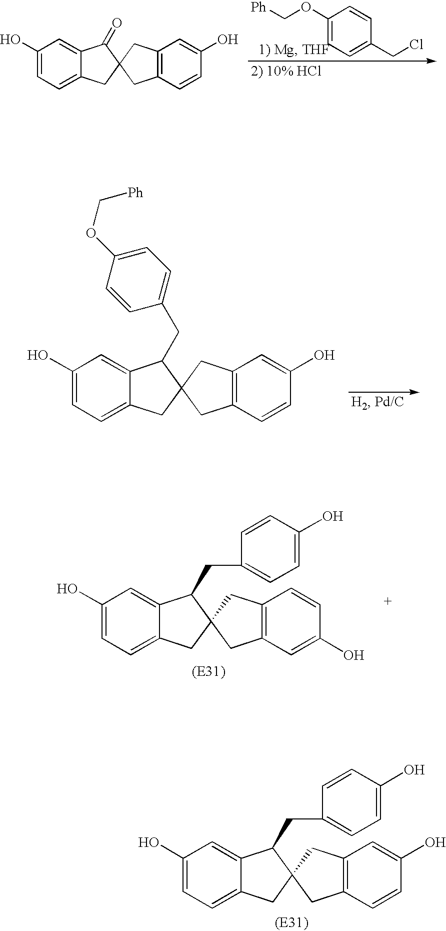

Step 1. Magnesium turning (240 mg, 10 mmol) was placed in a flame-dried flask and activated with a tiny crystal of iodine. 5 mL of dry THF was added and followed by slow addition of a solution of 4-methoxy benzyl chloride (2.33 g, 10 mmol) in 15 mL of dry THF. After stirring for 1 hr, a solution of 6,5′-dihydroxy-1,1′,3,3′-tetrahydro-2,2′-spirobi(2H-indene)-1-one (294 mg, 1 mmol) in 15 mL of dry THF was added into the flask at 0° C. under nitrogen The reaction mixture was stirred at room temperature for 3 hr and then treated with 10% HCl. After stirring for 1 hr, the mixture was extracted with ethyl acetate. The organic phase was washed with brine, dried (anhydrous magnesium sulfite) filtered and concentrated. The residue was purified by chromatography on silica gel eluted with ethyl acetate/light petroleum ether (1:4). Pure fractions were pooled and concentrated to give 197 mg (44%) of 6,5′-dihydroxy-1-(benzyloxy)benzylidene-1,1′,3,3′-tetrahydro-2,2′-spirobi(2H-indene) (E/Z=1.0:2.6). Preparative HPLC separation (C8 column, CH₃CN/NH₄OAc buffer, gradient) gave E6,5′-dihydroxy-1-(p-benzyloxy)benzylidene-1,1′3,3′-tetrahydro-2,2′-spirobi(2H-indene) and Z-6,5′-dihydroxy-1-(p-benzyloxy)benzylidene-1,1′,3,3′-tetrahydro-2,2′-spirobi(2H-indene). E-6,5′-dihydroxy-1-(p-benzyloxy)benzylidene-1,1′,3,3′-tetrahydro-2,2′-spirobi(2H-indene). ¹H NMR (CDCl₃): δ 7.41-7.27 (m, 5H), 7.11-7.05 (m, 2H), 7.02-6.96 (m, 3H), 6.94 (s, 1H), 6.79-6.75 (m, 2H), 6.74-6.59 (m, 3H), 4.99 (s, 2H), 3.54 (t, 2H), 2.98 (s, 2H), 2.78 (dd, 2H). LC-MS-Q+1: 447.1, LC-MS-Q−1: 445.3. Z-6,5′-dihydroxy-1-(p-benzyloxy)benzylidene-1,1′,3,3′-tetrahydro-2,2′-spirobi(2H-indene). ¹H NMR (acetone-D₆): δ 8.11 (s, OH), 8.09 (s, OH), 7.52-7.30 (m, 5H), 7.27-7.20 (m, 2H), 7.09-6.96 (m, 4H), 6.85-6.79 (m, 1H), 6.74-6.62 (m, 3H), 6.50 (s, 1H), 5.10 (s, 2H), 3.14 (t, 2H), 2.95-2.69 (m, 4H). LC-MS-Q+1: 447.1, LC-MS-Q−1: 445.3.

Step 2. A solution of (Z/E)6,5′-dihydroxy-1-(p-benzyloxy)benzylidene-1,1′,3,3′-tetrahydro-2,2′-spirobi(2H-indene) (20 mg, 0.045 mmol) and 10% palladium on carbon (10 mg) in 5 mL of methanol was hydrogenated under atmospheric pressure with stirring at room temperature overnight. The catalyst was removed by filtration (celite) and the filtrate was concentrated. The resulting residue was purified by preparative HPLC. (C8-column, CH₃CN/NH₄OAc buffer, gradient) to afford Rac-(1′R,2S/1′S,2R)-6,5′-dihydroxy-1-hydroxy)benzyl-1,1′,3,3′-tetrahydro-2,2′-spirobi(2H-indene) and Rac-(1′R,2R/1′S,2S)-6,5 ′-dihydroxy-1-(p-hydroxy)benzyl-1,1′,3,3′-tetrahydro-2,2′-spirobi(2H-indene). Rac-(1′R,2S/1′S,2R)-6,5′-dihydroxy-1-(p-hydroxy)benzyl-1,1′,3,3′-tetrahydro-2,2′-spirobi(2H-indene): ¹H NMR (acetone-D₆): δ 8.17 (s, OH), 8.06 (s, OH), 7.89 (s, OH), 6.97-6.91 (n, 4H), 6.74-6.70 (n, 3H), 6.60-6.55 (m, 2H), 6.17 (d, 1H), 3.20 (q 1H), 3.14 (d, 1H), 3.0 (dd, 1H), 2.82-2.76 (m, 2H), 2.72-2.54 (m, 4H). LC-MS-Q+1: 359.2, LC-MS-Q−1: 357.1. Rac-(1′R,2R/1′S,2S)-6,5′-dihydroxy-1-(p-hydroxy)benzyl-1,1′,3,3′-tetrahydro-2,2′-spirobi(2H-indene): ¹H NMR (acetone-D₆): 8.17 (s, OH), 8.05 (s, OH), 7.90 (s, OH), 6.98 (d, 1H, 6.97-6.93 (m, 3H), 6.74-6.71 (m, 2H), 6.64-6.55 (m, 3H), 6.17 (d, 1H), 3.22 (dd, 1H), 3.10 (d, 1H), 3.0 (dd, 1H), 2.84-2.78 (m, 2H), 2.69-2.53 (m, 4H), LC-MS-Q+1: 359.2, LC-MS-Q−1: 357.1.

### EXAMPLE 32

### Rac-1′R,2S/1′S,2R)-6,5-dihydroxy-1-[p-(2″-piperidinylethoxy)benzyl]-1,1′, 3,3′-tetrahydro-2,2′-spirobi(H-indene); and rac-(1′R,2R/1′S,2S)-6,5′-dihydroxy-1-[p(2″piperidinylethoxy)benzyl]-1,1′,3,3′-tetrahydro-2,2′-spirobi(2H-indene)

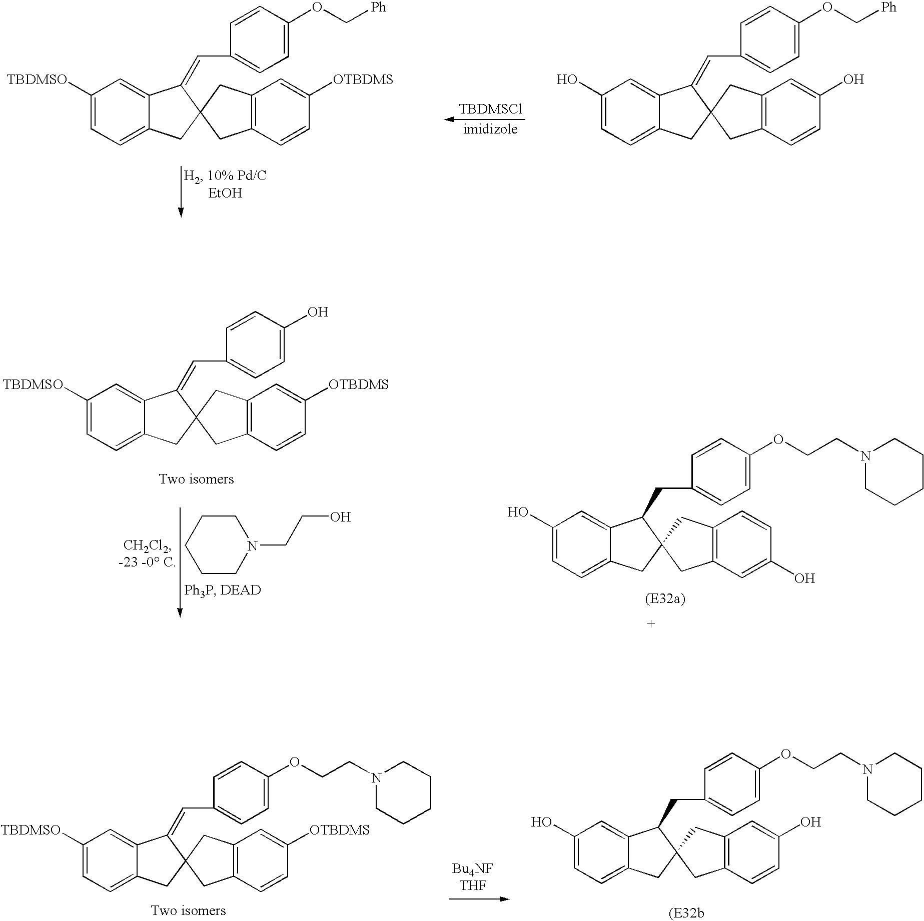

Step 1. A mixture of 6,5′-dihydroxy-1-(p-benzyloxy)benzylidene-1,1′,3,3′-tetrahydro-2,2′-spirobi(2H-indene) (224 mg, 0.5 mmol), t-butyldimethylsilyl chloride (166 mg, 1.1 mmol) and imidazole (136 mg, 2 mmol) in 10 mL of DMF was stirred under nitrogen at 130° C. overnight. The reaction mixture was partitioned between ethyl acetate and water. The organic phase was washed with brine, dried (anhydrous magnesium sulfate), filtered and concentrated. The residue was purified by a short silica gel column eluted with ethyl acetate/light petroleum ether (1:1). Pure fractions were pooled and concentrated affording 6,5′-di[(t-butyldimethyl)silyloxy]-1-(p-benzyloxy)benzylidene-1,1′,3,3′-tetrahydro-2,2′-spirobi(2H-indene).

Step 2. A mixture of (Z/E)-6,5′-di[(t-butyldimethyl)silyloxy]-1-(p-benzyloxy)benzylidene-1,1′,3,3′-tetrahydro-2,2′-spirobi(2H-indene) (54 mg, 0.08 mmol) and palladium on carbon (10%, 30 mg in 3 mL of methanol and 2 mL of ethyl acetate was stirred at room temperature under hydrogen atmosphere from a balloon. The reaction completed after 2 hr and the catalyst was removed by filtration (celite). The irate was concentrated to give Rac-6,5′-di[(t-butyldimethyl)silyloxy]-1-(p-hydroxybenzyl)-1,1′,3,3′-tetrahydro-2,2′-spirobi(2H-indene). ¹H NMR (CDCl₃): δ 7.03-6.89 (m, 4H), 6.73-6.67 (m, 3H), 6.62-6.54 (m, 3H), 3.19-2.95 (m, 3H), 2.84-2.46 (m, 6H), 0.97 (s, 9H), 0.90 (s, 9H), 0.17 (d, 6H), 0.03 (d, 6H). LC-MS Q+1: 587.5, LC-MS-Q−1: 585.4.

Step 3. A mixture of Rac-6,5′di[(t-butyldimethyl)silyloxy]-1-(p-hydroxybenzyl)-1,1′,3,3′-tetrahydro-2,2′-spirobi(2H-indene) (20 mg, 0.034 mmol), triphenyl phosphine (72 mg, 0.28 mmol) and N(2-hydroxyethyl)-piperidine (36 mg, 0.28 mmol) in 3 mL of dichloromethane was sired at −23° C. (dry ice/CCl₄) under nitrogen. To a solution was added a solution of diethyl azodicarboxylate (DEAD) (47 mg, 0.27 mmol) in 2 mL of dichloromethane at the above temperature. After addition, the reaction mixture was placed in refrigerator (0-4° C.) to stand overnight. The reaction was quenched by addition of saturated aqueous Ammonium chloride solution and the waster phase was extracted with ether (2×30 mL). The ether layer was combined and dried over anhydrous magnesium sulfate, filtered and concentrated. The residue was purified by preparative HPLC (C8-column, 280 nm⁻¹) with CH₃CN to give rac-6,5′-di[(t-butyldimethyl)silyloxy]-1-[p-2″-piperidinylethoxy)benzyl]-1,1′,3,3′-tetrahydro-2,2′-spirobi(2H-indene). ¹H NMR (CDCl₃): δ 7.03-6.91 (m, 4H), 6.84-6.75 (m, 2H, 6.72-6.54 (m, 3H), 6.06 (s, 1H), 4.07 (t, 2H), 3.24-2.64 (m, 11H), 2.58-2.46 (m, 4H), 1.66-1.54 (m, 4H), 1.48-1.36 (m, 2H), 0.96 (s 9H), 0.89 (s, 9H), 0.18 (d, 6H), 0.02 (d, 6H). LC-MS-Q+1: 698.7.

Step 4. Rac-6,5′-di[(t-butyl, dimethyl)silyloxy]-1-[p-(2″-piperidinylethoxy)benzyl]-1,1′,3,3′-tetrahydro-2,2′-spirobi(2H-indene) (12 mg, 0.017 mmol) was treated with 2 mL of 1M solution of tetrabutyl ammonium fluoride in dry THF. The solution was stirred at room temperature under nitrogen for 1 day. The reaction mixture was partitioned between aqueous saturated Ammonium chloride solution ethyl acetate (3×20 mL). The organic phase was dried (anhydrous magnesium sulfate), filtered and concentrated. The residue was purified by preparative HPLC (C8 column, NH₄OAc buffer/CH₃CN: gradient from 4:1 to 3:2) to give Rac-(1′R,2S/1′S, 2R)-6,5′-dihydroxy-1-[p-(2″-piperidinylethoxy)benzyl]-1,1′,3,3′-tetrahydro-2,2′-spirobi(2H-indene) and Rac-(1′R,2R/1′S,2S)-6,5′-dihydroxy-1-[p-(2″-piperidinylethoxy)benzyl]-1,1′,3,3′-tetrahydro-2,2′-spirobi(2H-indene) Rac-(1′R,2S/1′S,2R)-6,5′-dihydroxy-1-[2″-piperidinylethoxy)benzyl]-1,1′,3,3′-tetrahydro-2,2′-spirobi(2H-indene): ¹H NMR (acetone-D₆): δ 7.02 (d, 2H), 6.95-6.88 (, 2H), 6.83 (d, 2H), 6.67 (d, 1H), 6.60-6.53 (m, 2H), 6.10 (d, 1H), 4.46-4.39 (m, 2H), 3.30-3.22 (m, 2H), 3.18-2.54 (m, 13H), 1.92-1.77 (m, 4H), 1.63-1.50 (m, 2H). LC-MS-Q+1: 470.5, LC-MS-Q−1: 468.4. Rac-(1′R,2R/1′S,2S)-6,5′-hydroxy-1-[p-(2″-piperidinylethoxy)benzyl]-1,1′,3,3′-tetrahydro-2,2-spirobi(2H-indene): ¹H NMR (acetone-D₆): δ 7.02-6.90 (m, 4H), 6.84 (d, 2H), 6.62-6.54 (m, 3H), 6.14 (m, 1H), 4.47-4.39 (m, 2H), 3.35-3.28 (m, 2H), 3.20-2.54 (m, 13H), 1.93-1.81 (m, 4H), 1.66-1.51 (m, 2H). LC-MS-Q+1: 470.5, LC-MS-Q−1: 468.4.

### EXAMPLE 33

### 7′-Hydroxy-1,3,3,′4′-tetrahydro-spiro-[2H-indene-2,2′-(1′H)-naphthalene]-1′-one

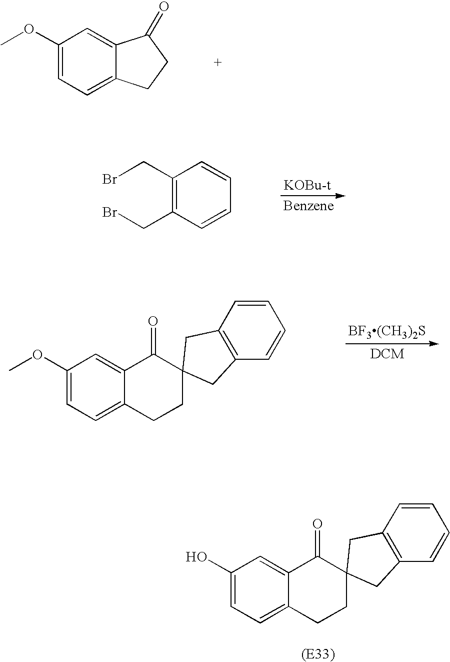

Step 1. A mixture of 7-methoxy-1-tetralone (3.52 g, 20 mmol), o-xylene dibromide (528 g, 20 mmol) and potassium t-butoxide (4.49 g, 40 mmol) in 100 mL of benzene was heated under reflux for 10 hr. The reaction mixture was partitioned between 10% hydrochloric acid and ethyl acetate. The organic phase was washed with water and brine, dried, filtered and concentrated. The resulting residue was purified by chromatography on silica gel eluted with ethyl acetate/toluene (5/95). Pure ions were pooled and concentrated affording 7′-methoxy-1,3,3′,4′-tetrahydro-spiro[2H-indene-2,2′-(1′H)-naphthalene]-1′-one.

Step 2. To the mixture of above compound (553 mg, 2 mmol) in 10 mL of dichloromethane was added 6 mL of boron trifluoride-methyl sulfide complex at 0° C. The mixture was stirred at room temperature overnight and then was treated with ice-water and ethyl acetate. The organic phase was washed with brine, dried (magnesium sulfate), filtered and concentrated. The residue was purified by chromatography on silica gel eluted with ethyl acetate/toluene (5/95). Pure fractions were pooled and concentrated to give 7′-hydroxy-1,3,3′,4′-tetrahydro-spiro[2H-indene-2,2′-(1′H)naphthalene]-1′-one. ¹H NMR (CDCl₃): δ 8.55 (s, OH), 7.46 (d, 1H), 7.26-7.01 (m, 6H), 3.34 (c, 2H), 3.06-2.87 (m, 4H), 2.13 (t, 2H).

### EXAMPLE 34

### 7′-Hydroxy-1,1′,3,3′,4′-pentahydro-spiro[2H-indene-2,2′-(1′H)-naphthalene]

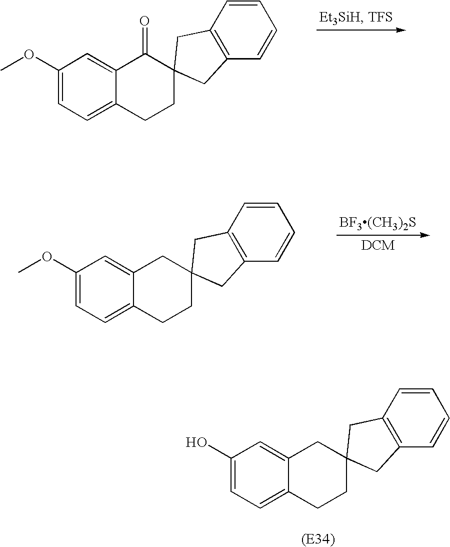

Step 1. A mixture of 7′-methoxy-1,3,3′,4′-tetrahydro-spiro[2H-indene-2,2′-(1′H)-naphthalene]-1′-one (258 mg, 0.93 mmol), triethylsilane (272 mg, 2.34 mmol) in 5 mL of TFA was stirred at room temperature for 4 days. TFA was removed by evaporation under vacuum. The resulting oil was partioned between ethyl acetate and saturated sodium bicarbonate solution and the organic phase was washed with brine, dried (anhydrous magnesium sulfate), filtered and concentrate. The residue was purified by column chromatography on silica gel eluted with benzene/heptane (3/7 affording 7′-methoxy-1,1′,3,3′,4′-pentahydro-spiro[2H-indene-2,2′-(1′H)-naphthalene]. ¹H NMR (acetone-D₆): δ 7.23-7.16 (m, 4H), 7.08 (d, 1H), 6.75 (dd, 1H), 6.58 (d, 1H), 3.78 (s, 3H), 2.96-2.85 (m, 4H), 2.83 (s, 2H), 2.77 (s, 2H), 10.91 (t, 2H).

Step 2. To the mixture of above compound (100 mg, 0.38 mmol) in 8 mL of dichloromethane, was added 2 mL of boron trifluoride methyl sulfide complex at 0° C. The mix re was stirred at room temperature under nitrogen for 2 days and then was treated with ice-water and ethyl acetate. The organic phase was washed with brine, dried (magnesium sulfate), filtered and concentrated. The residue was chromatographed on silica gel eluted with ethyl acetate/light petroleum ether (1/4). Pure fractions were pooled and concentrated, affording 7′-hydroxy-1,1′,3,3′,4′-pentahydro-spiro[2H-indene-2,2′-(1H)-naphthalene]. ¹H NMR (acetone-D₆): δ 8.04 (s, OH), 7.22-7.03 (m, 4H), 6.93 (d, 1H), 6.63 (dd, 1H), 6.47 (d, 1H), 2.88-2.75 (m, 4H), 2.72 (s, 2H), 2.62 (s, 2H), 1.82 (t, 2H).

### EXAMPLE 35

### 5,7′-Dihydroxy-1,3,3′,4′-tetrahydro-Spiro[2H-indene-2,2′-(1′H)-naphthalene]-1′-one

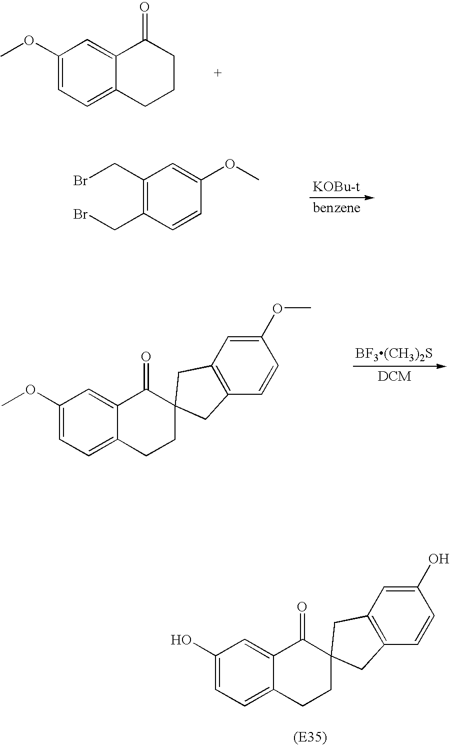

Step 1. A mixture of 7-methoxy-1-tetralon (2.29 g, 13 mmol), and 1,2-bis[bromomethyl]-4-methoxybenzene (3.82 g, 13 mmol) and potassium t-butoxid (2.92 g, 26 mmol) in 50 mL of benzene was stirred at 100° C. overnight. The reaction mixture was partitioned between 10% hydrochloric acid and ethyl acetate. The organic phase was washed with water and brine, dried, filtered and concentrated. The resulting residue was purified by chromatography on silica gel eluted with ethyl acetate/toluene (5/95). Pure fractions were pooled and concentrated affording 5,7′-dimethoxy-1,3,3′,4′-tetrahydro-spiro[2H-indene-2,2′-(1H-naphthalene]-1′-one.

Step 2. To the mixture of 5,7′-dimethoxy-1,3,3,4′-tetrahydro-spiro[2H-indene-2,2′-(1′H)-naphthalene]-1′-one (656 mg, 2.14 mmol) in 25 mL of dichloromethane, was added 8 mL of boron trifluoride-methyl sulfide complex at 0° C. The mixture was stirred at room temperature for 2 days and then was with ice-water and ethyl acetate. The organic phase was washed with brine, dried (magnesium sulfate), filtered and concentrated. The residue was chromatographed on silica gel eluted with ethyl acetate/light petroleum ether (1/8). Pure fractions were pooled and concentrated to give 5,7′-dihydroxy-1,3,3′,4′-tetrahydro-spiro[2H-indene-2,2′-(1′H)-naphthalene]-1′-one. ¹H NMR (DMSO-D₆): δ 9.58 (s, OH), 9.10 (s, OH), 7.24 (d, 1H), 7.14 (d, 1H), 6.97 (dd, 1H), 6.92 (d, 1H), 6.61-6.50 (m, 2H), 3.24-3.05 (m, 2H), 2.97-2.78 (m, 4H), 2.07 (t, 2H). GC-MS: 424.5 (TMSCl silylated).

### EXAMPLE 36

### 5-Hydroxy-7′-methoxy-1,3,3′,4′-tetrahydro-spiro[2H-indene-2,2′-(1′H)-naphthalene]-1′-one

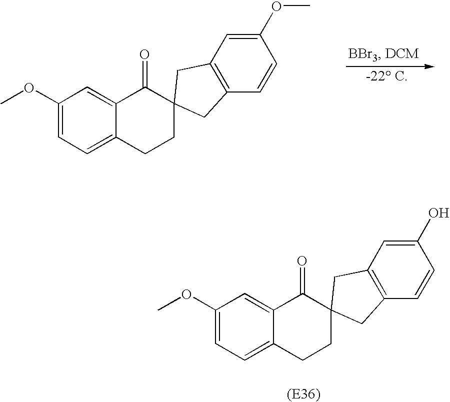

Boron tribromide (3.89 mL, 1 M in DCM) was added dropwise to the solution of 5,7′-dimethoxy-1,3,3′,4′-tetrahydro-spiro[2H-indene-2,2′-(1′H)naphthalene]-1′-one (1.0 g, 3.24 mmol) in 35 mL of dichloromethane at −78° C. The mixture was sired for 4 hr under nitrogen at −78° C., and allowed to stand in freezer (−78° C.) over 2 days. The reaction was monitored by TLC (35:65 EtOAc: here) and when complete the mixture was red with water and washed with brine. The organic phase was dried (anhydrous magnesium sulfate), filtered and concentrated. The residue was purified by column chromatography on silica gel eluted with ethyl acetate/heptane (20:80). Pure fractions were pooled and concentrated to yield 5-hydroxy-7′-methoxy-1,3,3,4′4′-tetrahydro-spiro[2H-indene-2,2′-(1′H)naphthalene]-1′-one. ¹H NMR (acetone-D6): ä 2.2 (m, 2H), 2.81 (d, 1H), 2.87 (d, 1H), 3.0 (m, 2), 3.25 (d, 1H), 3.31 (d, 1H), 3.81 (s, 3H), 6.56 (dd, 1H), 6.61 (d, 1H), 6.93 (d, 1H), 7.12 (dd, 1H), 7.25 (d, 1H), 7.43 (d, 1H), 8.06 (s, 1H). LC-MS-Q+1: 295.3, LC-MS-Q−1: 293.5.

### EXAMPLE 37

### 5,7′-Dihydroxy-1,3,3′,4′-tetrahydro-spiro[2H-indene-2,2′-(1′H)-naphthalene]

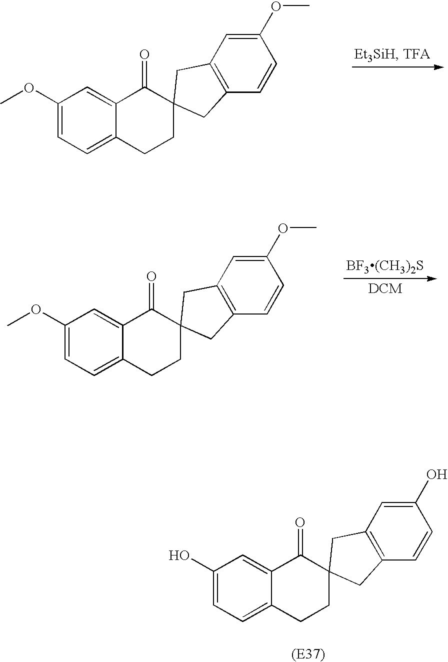

Step 1. A mixture of 5,7′-dimethoxy-1,3,3′,4′-tetrahydro-spiro[2H-indene-2,2′-(1′H)-nwaphthalene]-1′-one (656 mg, 2.14 mmol), triethylsilane (622 mg, 5.35 mmol) in 10 mL of TFA was stirred at room temperature for 4 days. TFA was removed by evaporation under vacuum. The resulting oil was partitioned between ethyl acetate and saturated sodium bicarbonate solution and the organic phase was washed with brine, dried (anhydrous magnesium sulfate), filtered and concentrated. The residue was purified by column chromatography on silica gel eluted with benzene/heptane (3/7) affording 5,7′-dimethoxy-1,3,3′,4′-tetrahydro-spiro[2H-indene-2,2′-(1′H)-naphthalene]. ¹H NMR (CDCl₃): δ 7.11-7.04 (m, 2H), 6.79-6.69 (m, 3H), 6.57 (d, 1H), 3.80 (s, 3H), 3.77 (s, 3H), 2.92-2.84 (m, 3H), 2.82-2.68 (n, 5H), 1.90 (t, 2H). GC-MS: 293.9.

Step 2. To the mixture of above compound (140 mg, 0.48 mmol) in 10 mL of dichloromethane, was added 2 mL of boron trifluoride-methyl sulfide complex at 0° C. The mixture was sired at room temperature for 3 days and then was treated with ice-water and ethyl acetate. The organic phase was washed with brine, dried (magnesium sulfate), filtered and concentrated. The residue was chromatographed on silica gel eluted with ethyl acetate/light petroleum ether (1/4). Pure actions were pooled and concentrated, affording 5,7′-dihydroxy-1,3,3′,4′-tetrahydro-spiro[2H-indene-2,2′-(1′H)-naphthalene]. ¹H NMR (CD₃OD): δ 6.91-6.83 (m, 2H), 6.61-6.50 (m, 3H), 6.39 (d, 1H), 2.72 (t, 2H), 2.67-2.49 (m, 4H), 2.55 (s, 2H), 1.73 (t, 2H).

### EXAMPLE 38

### 5,7′-Dihydroxy-1′-methyl-1,3,3′4′-tetrahydro-spiro[2H-indene-2,2′-(1′H)-naphthalene]

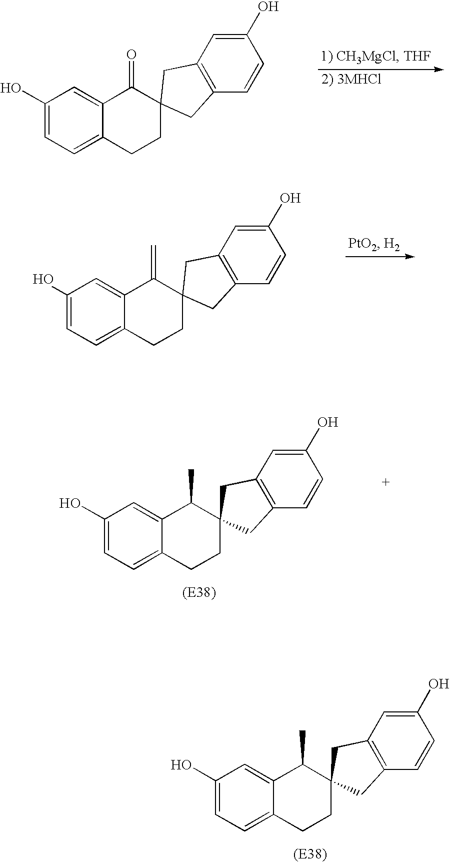

Step 1. A solution of 5,7′-dihydroxy-1,3,3′,4′-tetrahydro-spiro[2H-indene-2,2′-(1′H)-naphthalene]-1′-one (100 mg, 0.38 mmol) in 10 mL of anhydrous THF was treated with CH₃MgCl (1.6 mL, 4.8 mmol) at 0° C. The reaction mixture was stirred for 48 hr at room temperature and then was treated with 3M HCl. After sting for 3 hr, the mixture was partitioned between ethyl acetate and water. The organic phase was washed with brine, dried (anhydrous magnesium sulfate), filtered and concentrated. The residue was purified by column chromatography on silica gel eluted with ethyl acetate/heptane (30:70). Pure fractions were pooled and concentrated, affording 5,7′-dihydroxy-1,3,3′,4′-tetrahydro-spiro[2H-indene-2,2′-(1′H)-naphthalene]-1′-methylidene. ¹H NMR (acetone-D6): ä 1.87-1.96 (m, 2H), 2.77-2.96 (m, 6H), 4.93 (s, 1H), 5.34 (s, H), 6.58 (dd, 1H), 6.73 (d, 1H), 6.74 (dd, 1H), 6.93 (m, 2H), 7.05 (d, 1H). LC-MS-Q+1: 279.4, LC-MS-Q−1: 277.3.

Step 2. A mixture of 5,7′ dihydroxy-1,3,3′,4′-tetahydro-spiro[2H-indene-2,2′-(1′H)-naphtialene]-1′-methylidene (10 mg, 0.04 mmol) and PtO₂ (10 mg) in 5 mL of ethyl acetate was stirred under an atmosphere of hydrogen for 1 hr. The catalyst was removed by filtration through celite and the filtrate was concentrated and purified by preparative HPLC, affording two diastereomers of 5,7′-dihydroxy-1′-methyl-1,3,3′,4′-tetraydro-spiro[2H-indene-2,2′-(1′H)naphthalene].

Rac-(1′S,2R/1′R,2S)-5,7′-dihydroxy-1′methyl-1,3,3′,4′-tetrahydro-spiro[2H-indene-2,2′-(1′H)-naphthalene] ¹H NMR (acetone-D6): ä 1.15 (d, 3H), 1.62 (m, 1H), 1.85 (m, 1H), 2.43 (d, 1H), 2.55 (d, 1H), 2.64-2.80 (m, 4H), 2.97 (d, 1H), 6.55-6.62 (m, 3H), 6.67 (d, 1H), 6.8 (d, 1H), 6.91 (d, 1H). LC-MS-Q: 279.1.

Rac-(1′S,2S/1′R,RS)-5,7′-dihydroxy-1′methyl-1,3,3′,4′-tetrahydro-spiro[2H-indene-2,2′-(1′H)-naphthalene]. ¹H NMR (acetone-D6): ä 1.15 (d, 3H), 1.62 (m, 1H), 1.85 (m, 1H), 2.45 (d, 1H), 2.55 (d, 1H), 2.70 (q, 1H), 2.74-2.82 (m, 3H), 2.90 (d, 1H), 6.58-6.62 (m, 4H), 6.9 (d, 1H), 6.97 (d, 1H). LC-MS-Q−1: 279.1.

### EXAMPLE 39

### 6′-Hydroxy-1,3,3′,4′-tetrahydro-spiro[2H-indene-2,2′-(1′H)-naphthalene]-1′-one

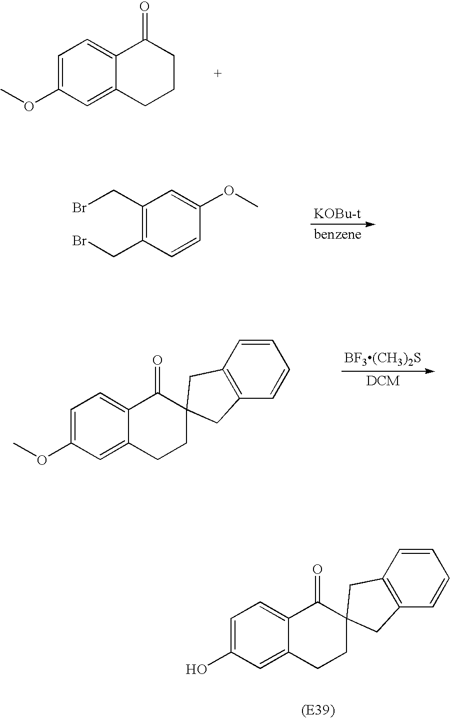

Step 1. A mixture of 6-methoxy-1-tetralone (3.52 g, 20 mmol), o-xylene dibromide (5.28 g, 20 mmol) and potassium t-butoxide (4.49 g, 40 mmol) in 100 mL of benzene was heated under reflux for 2 days. The reaction mixture was treated with 10% hydrochloric acid and the benzene phase was separated. The organic phase was washed with water and brine, dried, filtered and concentrated. The resulting residue was purified by chromatography on silica gel eluted with ethyl acetate/toluene (5/95). Pure fractions were pooled and concentrated affording 6′-methoxy-1,3,3′,4′-tetrahydro-spiro[2H-indene-2,2′-(1′H)-naphthalene]-1′-one that can be crystallized from methanol to give white crystals.

Step 2. To a mixture of 6′-methoxy-1,3,3′,4′-tetrahydro-spiro[2H-indene-2,2′-(1′H)-naphthalene]-1′-one (320 mg, 1.15 mmol) in 10 mL of dichloromethane, was added 5 mL of boron trifluoride-methyl sulfide complex at −78° C. The mixture was stirred at room temperature under nitrogen for 1 days and then was treated with ice-water. The organic phase was washed with brine, dried (magnesium sulfate), filtered and concentrated. The residue was chromatographed on silica gel eluted with ethyl acetate/light petroleum ether (1/4). Pure fractions were pooled and concentrated, which was crystallized from methanol and petroleum ether, affording 6′-hydroxy-1,3,3′,4′-tetrahydro-spiro[2H-indene-2,2′-(1′H)-naphthalene]-1′-one. ¹H NMR (acetone-D₆): δ 9.17 (s, OH), 7.86 (d, 1H), 7.24-7.08 (m, 4H), 6.81 (dd, 1H), 6.72 (d, 1H), 3.38 (d, 2H), 3.05-2.96 (m, 4H), 2.15 (t, 2H).

### EXAMPLE 40

### 6′-Hydroxy-1,1′,3,3′,4′-pentahydro-spiro [2H-indene-2,2′-(1′H)-naphthalene]

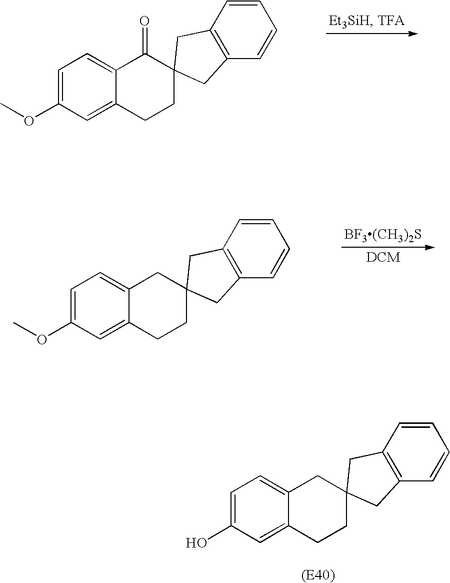

6′-methoxy-1,3,3′,4′-tetrahydro[2H-indene-2,2′-(1′H)-naphthalene]-1′-one (553 mg, 2 mmol), triethylsilane (581 mg, 5 mmol) in 10 mL of TFA was stirred at room temperature for 3 days. TFA was removed by evaporation under vacuum. The resulting oil was partitioned between ethyl acetate and saturated sodium bicarbonate solution and the organic phase was washed with brine, dried (anhydrous magnesium sulfate), filtered and concentrated. The residue was purified by column chromatography on silica gel eluted with benzene/heptane (3/7) affording 260 mg (50%) of white solid. To the mixture of above compound (130 mg, 0.5 mmol) in 10 mL of dichloromethane, was added 4 mL of boron trifluoride-methyl sulfide complex at −78° C. The mixture was stirred at room temperature under nitrogen for 4 days and then was treated with ice-water. The organic phase was washed with brine, dried (magnesium sulfate), filtered and concentrated. The residue was chromatographed on silica gel eluted with 5% ethyl acetate in toluene. Pure fractions were pooled and concentrated, affording 6-hydroxy-1,1′,3,3′,4′-pentahydro-spiro[2H-indene-2,2-(1′H)-naphthalene]. ¹H NMR (acetone-D₆):δ 7.99 (s, OH), 7.17-7.04 (m, 4H), 6.78 (d, 1H), 6.65-6.56 (m, 2H), 2.85-2.70 (m, 6H), 2.59 (s, 2H), 1.80 (t, 2H). GC-MS: 250.0.

### EXAMPLE 41

### 5,6′-Dihydroxy-1,3,3′,4′-tetrahydro-spiro[2H-indene-2,2′-(1′H)-naphthalene]-1-one

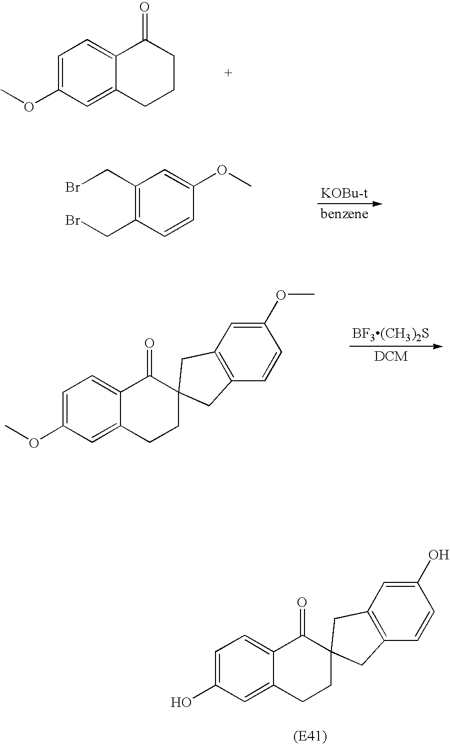

Step 1. To a solution of and 1,2-bis[bromomethyl]methoxybenzene (4.7 g, 16 mmol) and 6-methoxy-1-tetralone (2.8 g, 16 mmol) in 60 mL of benzene, was added potassium t-butoxide (4.0 g, 35 mmol) in portions. The mixture was stirred at room temperature for 2 hr and the reaction monitored by TLC (35:65 EtOAc: heptane). The reaction mixture was washed with water, brine, and the organic phase was dried (anhydrous a es sulfate), filtered and concentrated. The residue was purified by column chromatography on silica gel eluted with ethyl acetate/heptane (12:88) to yield 5,6′-dimethoxy-1,3,3′,4′-tetrahydro-spiro[2H-indene-2,2′-(1′H)-naphthalene]-1′-one.

Step 2. To a solution of 5,6′-dimethoxy-1,3,3′,4′-tetrahydro-spiro[2H-indene-2,2′-(1′H)-naphtbalene]-1′-one (1.8 g, 5.8 mmol) in 100 mL of dichloromethane, was added dropwise 17 mL of boron trifluoride/methyl sulfide complex. The reaction mixture was stirred at room temperature for 4 days under nitrogen. The reaction monitored by TLC (35:65 EtOAc:heptane) and when complete the mixture washed with water, brine, and the organic phase was dried (anhydrous magnesium sulfate), filtered and concentrated. The residue was purified by column chromatography on silica gel eluted with ethyl acetate/heptane (30:70) to yield 5,6′-dihydroxy-1,3,3′,4′-tetrahydro-spiro[2H-indene-2,2′-(1′H)-naphthalene]-1′-one. ¹H NMR (acetone-D₆): ä 1.9-2.2 (m, 2H), 2.8 (d, 1H), 2.85 (d, 1H), 3.0 (m, 2H), 3.15 (d, 1H), 3.35 (d, 1H), 6.55-6.75 (m, 3H), 6.8 (dd, 1H), 6.95 (d, 1H), 7.85 (d, 1H). LC-MS-Q+1: 281.2, LC-MS-Q−1: 279.1.

### EXAMPLE 42

### 5,6′-Dihydroxy-1,3,3′,4′-tetrahydro-spiro[2H-indene-2,2′-(1-naphthalene]

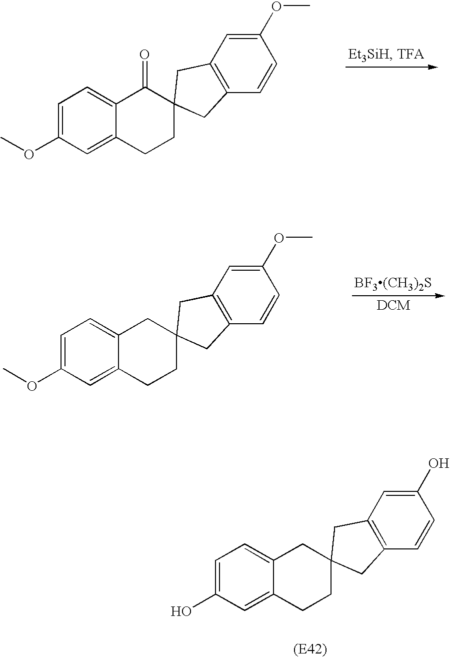

Step 1. A solution of 5,6′dimethoxy-1,3,3′,4′-tetrahydro-spiro[2H-indene-2,2′-(1′H)-naphthalene]-1′-one (500 mg, 1.62 mmol) in 6 mL of TFA was treated with Et₃SiH (0.75 g, 5.8 mmol) and the mixture was stirred for 24 hr at room temperature. The reaction mixture was evaporated to dryness and ethyl acetate was added. The organic phase was washed with aqueous sodium bicarbonate and brine, dried (anhydrous magnesium sulfate), filtered and concentrated. The residue was purified by column chromatography on silica gel eluted with ethyl acetate/light petroleum ether (1:8) to yield 400 mg (84%) of 5,6′-dimethoxy-1,3,3′,4′-tetrahydro-spiro[2H-indene-2,2′-(1′H)-naphthalene].

Step 2. To a solution of 5,6′-dimethoxy-1,3,3′,4′-tetrahydro-spiro[2H-indene-2,2′-(1′H)-naphthalene] (300 mg, 1.02 mmol) in 10 mL of dichloromethane, was added 5 mL of boron trifluoride/methyl sulfide complex dropwise. The mixture was stirred for 48 hr under nitrogen. The reaction was monitored by TLC (1:3 EtOAc: p-ether) and when complete the mixture was washed with water and then brine. The organic phase was dried (anhydrous magnesium sulfate), filtered and concentrated. The resulting residue was purified by column chromatography on silica gel eluted with ethyl acetate/light petroleum ether (1:3). Pure fractions were pooled and concentrated to yield 5,6′-dihydroxy-1,3,3′,4′-tetrahydro-spiro[2H-indene-2,2′-(1′H)-naphthalene]. ¹H NMR (acetone-D₆): ä 1.8 (m, 2H), 2.59-2.81 (m, 8H, 6.55-6.65 (m, 4H), 6.81 (d, 1H), 6.93 (d, 1H), 7.97 (s, 1H). LC-MS-Q: 265.0.

### EXAMPLE 43

### 5-Hydroxy-6′-methoxy-1,3,3′,4′-tetrahydro-spiro[2H-indene-2,2′-(1′H)-naphthalene]1′-one

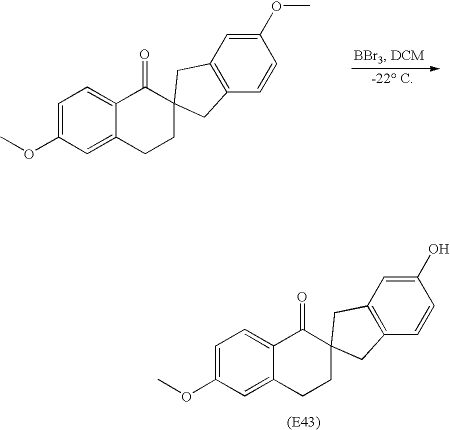

Boron tribromide (5.4 mL, 1 M in DCM) was added dropwise to a solution of 5,6′dimethoxy-1,3,3′,4′-tetrahydro-spiro[2H-indene-2,2′-(1′H)-naphthalene]-1′-one (1.0 g) in 40 mL of dichloromethane at −78° C. The mixture was stirred for 4 hr under nitrogen at −78° C. and allowed to stand in (−22° C.) over 3 days. The reaction mixture was with water and brine and the organic phase was dried (magnesium sulfate), filtered and concentrated. The residue was purified by column chromatography on silica gel eluted with ethyl acetate/heptane (20:80). Pure factions were pooled and concentrated to yield 5-hydroxy-6′-methoxy-1,3,3′,4′-tetrahydrospiro[2H-indene-2,2′-(1H)naphthalene]-1′-one. ¹H NMR (acetone-D₆): ä 2.12 (m, 2H),2.87 (m, 2H), 3.06 (, 2H), 3.18 (d, 1H), 3.31 (d, 1H), 3.87 (s, 3H), 6.56 (dd, 1H), 6.62 (d, 1H), 6.81 (d, 1H), 6.87 (dd, 1H), 6.93 (d, 1H), 7.87 (d, 1H), 8.06 (s, 1). LC-MS-Q+1: 295.3, LC-MS-Q−1: 293.5.

### EXAMPLE 44

### 5,6′-Dihydroxy-1′-methyl-1,3,3′,4′-tetrahydro-spiro[2H-indene-2,2′-(1′H)-naphthalene]

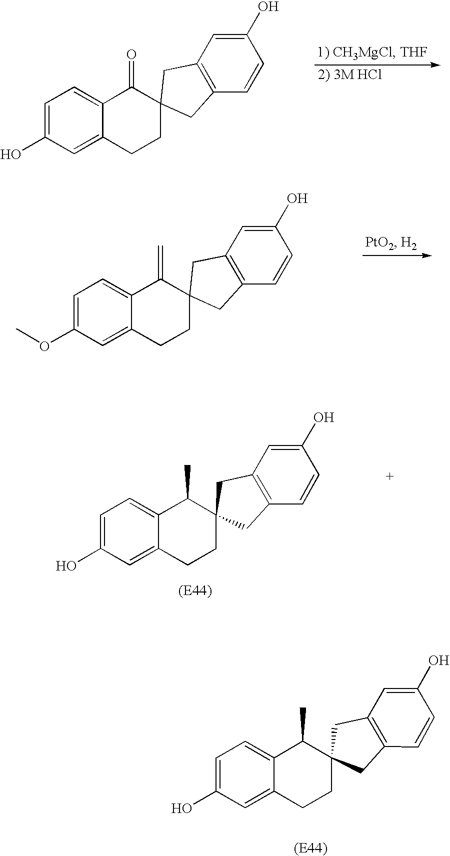

Step 1. A solution of 5,6′ dihydroxy-1,3,3′,4′-tetrahydr-spiro[2H-indene-2,2′-(1′H)-naphthalene]-1′-one (100 mg, 0.36 mmol) in 5 mL of anhydrous THF was treated with CH₃MgCl (3 mL, 20% in THF) at −70° C. The reaction mixture was stirred overnight at room temperature and then was treated with 10% HCl. The mixture was partioned between ethyl acetate and water. The organic phase was washed with brine, dried (anhydrous magnesium sulfate), filtered and concentrated. The residue was purified by column chromatography on silica gel eluted with ethyl acetate/heptane (30:70). Pure fractions were pooled and concentrated affording 5,6′ dihydroxy-1,3,3′,4′-tetrahydro-spiro[2H-indene-2,2′-(1′H)-naphthalene]-1′-methylidene. ¹H NMR (acetone-D6): ä 1.91 (t, 2H), 2.74-3.04 (m, 6H), 4.82 (s, 1), 5.27 (s, 1H), 6.56-6.70 (m, 4H), 6.95 (d, 1H), 7.41 (d, 1H), 7.94 (s, 1H), 8.39 (s, 1H). GC-Ms: 423.1 (silylated by TMSCl).

Step 2. A mixture of 5,6′-dihydroxy-1,3,3′,4′-terhydro-spiro[2H-indene-2,2′-(1′H)-naphthalene]-1′-methylidene (40 mg, 0.14 mmol) and PtO₂ (10 mg) in 5 mL of ethyl acetate was stirred under an atmosphere of hydrogen for 1 day. The catalyst was removed by filtration through celite and the filtrate was concentrated and purified by preparative HPLC affording two diastereomers of 5,6 ′-dihydroxy-1′-methyl-1,3,3′,4′-tetrahydro-spiro[2H-indene-2,2′-(1′H)-naphthalene]. Rac-(1′S,2R/1′R,2S)-5,6′-dihydroxy-1′methyl-1,3,3′,4′-tetrahydrospiro[2H-indene-2,2′-(1′H)-naphthalene]. ¹HNMR (CD₃OD): ä 1.15 (d, 3H), 1.62 (m, 1H), 1.85 (m, 1H), 2.43 (d, 1H), 2.55 (d, 1H), 2.64-2.80 (m, 4H), 2.97 (d, 1H), 6.55-6.62 (m, 3H), 6.67 (d, 1H), 6.8 (d, 1H), 6.91 (d, 1H). GC-MS: 424.2 (silylated by TMSCl) Rac-(1′S,2S/1′R,RS)-5,6′-dihydroxy-1′-methyl-1,3,3′,4′-tahydro-spiro[2H-indene-2,2′-(1′H)-naphthalene]. ¹H NMR (CD₃OD): ä 1.15 (d, 3M, 1.62 (m, 1H), 1.85 (n, 1H), 2.45 (d, 1H), 2.55 (d, 1H), 2.70 (q, 1H), 2.74-2.82 (m, 3H), 2.90 (d, 1H), 6.58-6.62 (m, 4H), 6.9 (d, 1H), 6.97 (d, 1H). LC-MS-Q+1: 281.5.

### EXAMPLE 45

### 1′-Substituted analogues of 5,6′-dihydroxy-1,3,3′,4′-tetrahydro-spiro[2H-indene-2,2′-(1′H)-naphthalene]-1′-one

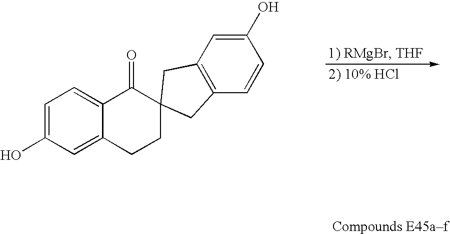

The following procedure was used for parallel synthesis of compounds E45a-f. The reactions were carried out in a Radley carousel reaction station equipped with 25 mL glass tube with reflux head and inert gas lines. 5,6′-dihydroxy-1,3,3′,4′-adrospiro[2H-indene-2,2′-(1′H)-naphthalene]-1′-one (50 mg, 0.178 mmol) was dissolved in 8 mL of anhydrous THF. The Grignard reagent (10 eq. in THF) was added at −70° C. under inert atmosphere. The reaction mix was allowed to stand at room temperature overnight 3 mL of 10% HCl was then added until the mixture was acidic. After 20 hr, the reaction mixture was extracted with ethyl acetate. The organic phase was washed with brine 3 times, dried by gravity flow through a short Na₂SO₄-column and evaporated under vacuum. The resulting residue was purified by preparative HPLC (see Table I). Method A: Isocratic run with 45% acetonitrile and 55% 10 mM ammonium ate water buffer. Flow rate: 12 mL/min. Method B: Isocratic run with 72% 10 mM formic acid water buffer and 28% acetonitrile.

E45a: 5,6′-dihydroxy-1′-ethylidene-1,3,3′,4′-tetrahydro-spiro[2H-indene-2,2′-(1′H)-naphthalene]. ¹H NMR (acetone-D₆): ä 7.14 (d, 1H), 6.94 (d, 1H), 6.67-6.64 (m, 3H), 6.58 (dd, 1H), 5.53 (q, 1H), 2.95 (d, 1H), 2.90 (d, 1H), 2.66 (d, 1H), 2.64 (m, 2H), 2.63 (d, 1H), 1.84-1.81 (m, 5H). LC-MS-Q−1: 291.1.

E45b: 5,6′-dihydroxy-1′-isopropylidene-1,3,3′,4′-tetrahydro-spiro[2H-indene-2,2′-(1′H)-naphthalene]. ¹H NMR (CD₃OD): ä 7.0 (d, 1H), 7.1 (d, 1H), 6.5 (m, 4H), 2.88 (d, 1H), 2.82 (d, 1H), 2.5 (m, 2H), 1.9 (s, 3H), ˜1.0 m, 2H), 1.75 (s, 3H), 1.5 (m, 2H). LC-MS-Q+1: 307.3″.

E45c: (Z)-5,6′-dihydroxy-1′-propylidene-1,3,3′,4′-4tetrahydro-spiro[2H-indene-2,2′-(1′H)-naphthalene]. ¹H NMR (acetone-D₆): ä 7.09 (d, 1H), 6.95 (d, 1H), 6.66-6.63 (m, 3H), 6.58 (dd, 1H), 5.38 (t, 1H), 2.97 (d, 1H), 2.92 (d, 1H), 2.66 (d, 1H), 2.64 (in, 2H), 2.63 (d, 1H), 2.28 (m, 2H), 1.82 (m, 2H), 0.99 (t, 3H). LC-MS-Q−1: 305.2.

E45d: (E5,6′-dihydroxy-1′-propylidene-1,3,3′,4′-ytydro-piro[2H-indene-2,2′-(1′H)-naphthalene]. ¹H NMR (acetone-D₆): ä 7.36 (d, 1H), 7.02 (d, 1H), 6.73-6.67 (m, 2H), 6.66 (dd, 1H), 6.59 (d, 1H), 5.77 (t, 1H), 3.29 (d, 1H), 3.26 (d, 1H), 2.95 (d, 1H), 2.87 (d, 1H), 2.58 (m, 2H), 2.08-2.04 (m, 2H), 1.63 (m, 2H), 1.03-0.96 (m, 3H). LC-MS-Q−1: 305.5.

E45e: (1R,2S)- and (1S,2R)-5,1′,6′-trihydroxy-1′-phenyl-1,3,3′,4′-tetrahydro-spiro[2H-indene-2,2′-(1′H)-naphthalene]. ¹H NMR (CDCl₃) ä 7.3-7.2 (m, 5H), 7.1 (d, 1H), 7.0 (d, 1H), 6.80-6.75 (m, 2H), 6.55-6.50 (m, 2H), 3.5 (d, 1H), 3.1 (d, 1H), 2.95 (m, 1H), 2.82 (m, 1H), 2.7 (d, 1H), 2.1 (d, 1H), 1.8 (m, 2H). LC-MS-Q−1: 357.1.

E45f: (1R,2R)- and 1S,2S)-5,1′,6′-trihydroxy-1′-phenyl-1,3,3′,4′-tetrahydro-spiro[2H-indene-2,2′-(1H)-naphthalene]. ¹H NMR (acetone-D₆): ä 7.32 (m, 2H), 7.22 (m, 2H), 7.18 (m, 1H), 7.05 (d, 1H), 6.9 (m, 2H), 6.72 (dd, 1H), 6.6 (d, 1H), 6.52 (dd, 1H), 3.5 (d, 1H), 3.2 (d, 1H), 3.0 (m, 1H), 2.8 (ddd, 1H), 2.6 (d, 1H), 1.78 (d, 1H), 1.74 (m, 2H). LC-MS-Q−1: 357.1.

TABLE I

| | | | purity (%) | |
|---|---|---|---|---|
| | | Grignard | C8-HPLC | HPLC |
| Entry | structure | reagent | (254 nm) | Method |
| E45a | 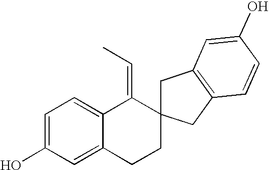 | EtMgBr | 98% for mixture of two isomers Z:E = 90:10 | A |
| E45a | 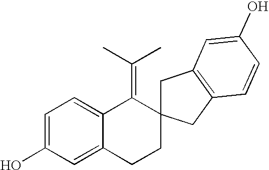 | i-PrMgBr | 97 | A |
| E45b | 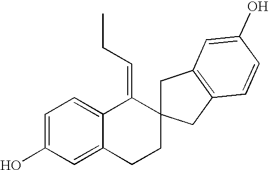 | PrMgBr | 94 | A |
| E45c | 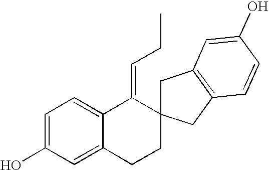 | PrMgBr | 57% impurites are non spiro Z-isomer <1% | A |
| E45d | 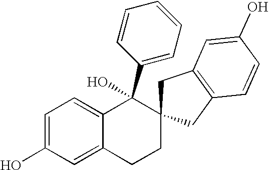 | PhMgBr | >95 | B |
| E45c | 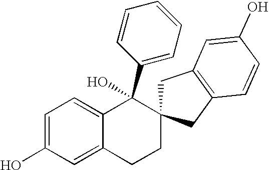 | PhMgBr | >95 | B |

### EXAMPLE 46

### 5,6′-Dihydroxy-1′-(p-methoxy)benzylidene-1,3,3′,4′-tetrahydro-spiro[2H-indene-2,2-(1′H)-naphthalene]

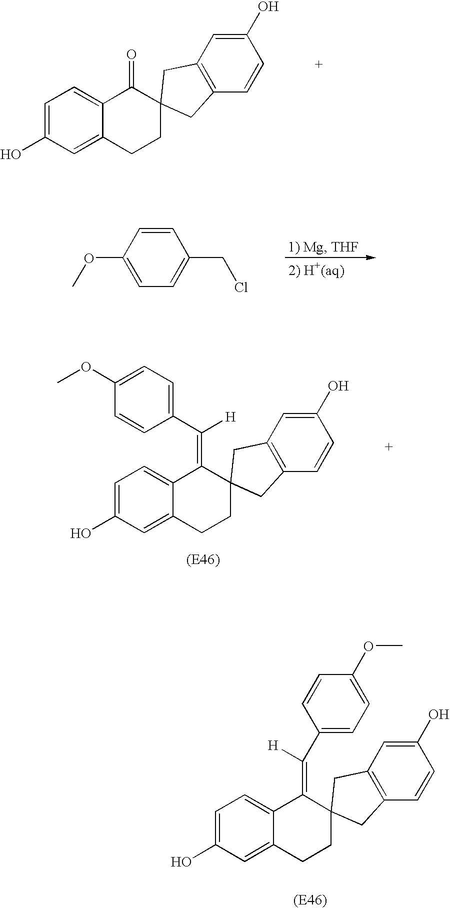

magnesium (870 mg, 35.6 mmol) and a few crystals of iodine in a oven-dried flask were heating under stirring and nitrogen. After iodine vapor disappears, anhydrous THF (1 mL) was added, followed by dropwise addition of 4-methoxybenzyl chloride (5.58 g, 35.6 mmol) dissolved in anhydrous THF (15 mL). The suspension was cooled to 0° C. and a solution of 5,6′-dihydroxy-1,3,3′,4′-tetrahydro-spiro[2H-indene-2,2′-(1′)naphthalene]-1′-one (500 mg, 1.78 mmol) in anhydrous THF (10 ml) was added. The mixture was for 48 h at room temperature. The reaction monitored by TLC (35:65=EtOAc:heptane) and when complete the r was treated with 3M HCl for 3 br, washed with water, brine, and the organic phase was dried with anhydrous magnesium sulfate, and concentrated. The product was isolated from the residue by preparative HPLC, affording the corresponding Z and E isomers of 5,6′-dihydroxy-1′-(p-methoxy)benzylidene-1,3,3′,4′-tetrahydro-spiro[2H-indene-2,2′-(1′H)-naphthalene]. E-5,6′-dihydroxy-1′-(methoxy)benzylidene-1,3,3′,4′-tetrahydro-spiro[2H-indene-2,2′-(1′H)-naphthalene]. ¹H NMR (acetone-D₆): ä 1.41 (m, 1H), 2.84 (m, 1H), 2.38 (d, 1H), 2.44 (d, 1H), 2.56-2.63 (m, 2H), 2.75 (m, 1H), 3.25 (m, 1H), 3.82 (s, 3H), 6.64-6.68 (m, 2H), 6.72-6.78 (m, 2H), 6.87 (m, 2H), 7.03 (d, 1H), 7.06 (s, 1H), 7.10 (m, 3H), 7.87 (d, 1H).

Z-5,6′-dihydroxy-1′-(p-methoxy)benzylidene-1,3,3′,4′-tetrahydro-spiio[2H-indene-2,2′-(1′H)-naphthalene). ¹HNMR (acetone-D₆): ä 1.46 (m, 1H), 1.75 (m, 1H), 2.37 (d, 1H), 2.43 (d, 1H), 2.56 (d, 1H), 2.59 (d, 1H), 2.75 (m, 1H), 3.25 (m, 1H), 6.65 (d, 1H), 6.68-6.78 (m, 3H), 6.81-6.87 (m, 2H), 7.03-7.12 (m, 4H), 7.79 (d, 1H). LC-MS-Q+1: 385.3, LC-MS-Q−1: 383.2

### EXAMPLE 47

### 6′-Methoxy-5-(2 ″-piperidinylethoxy)-1,3,3′,4′-tetrahydro-spiro 2H-indene-2,2′-(1′H)-naphthalene]-1′-one

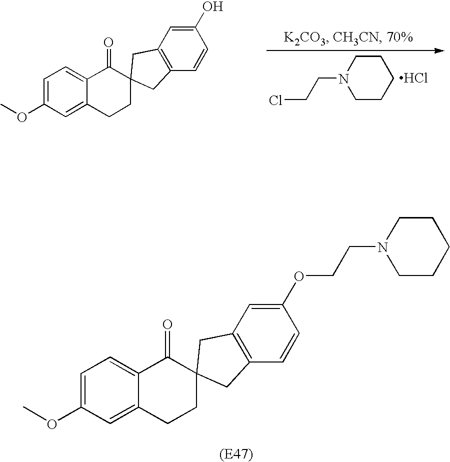

To a solution of 5-hydroxy-6′-methoxy-1,3,3′,4′-tetrahydro-spiro[2H-indene-2,2′-(1′H)-naphthalene]-1′-one (500 mg, 1.7 mmol) in acetonitrile (35 mL) was added K₂CO₃ (939 mg, 6.8 mmol) and N-(2-chloroethyl)-piperidine hydrochloride (938 mg, 5.1 mmol). The mixture was stirred for 24 hr at 82° C. The reaction monitored by TLC (2% solution of Et₃N in ether) and when complete the mixture washed with water, and the organic phase was dried with anhydrous magnesium sulfate, and concentrated. The residue was purified by column chromatography on silica gel (2% Et₃N in ether) to yield 6′-methoxy-5-(2″-piperidinylethoxy)-1,3,3′,4′-tetrahydro-spiro[2H-indene-2,2′-(1′H)-naphthalene]-1′-one

### EXAMPLE 48

### 6′-Hydroxy-5-(2″-piperidinylethoxy)-1,3,3′,4′-tetrahydro-spiro[2H-indene-2,2′(1′H)-naphthalene]-4 one

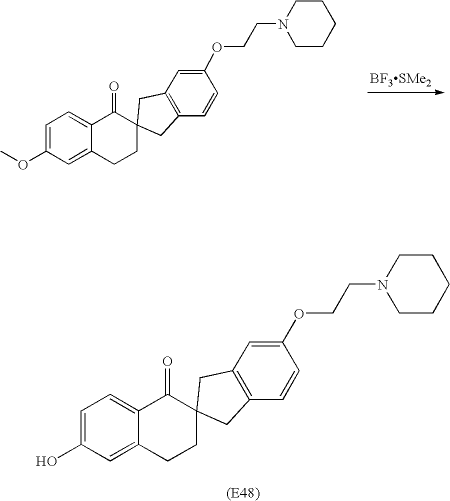

A solution of 6′-methoxy-5-(2′-piperidinylethoxy)1,3,3′,4′-tetrahydrospiro[2H-indene-2,2′ (1′H)-naphthalene]-1′-one (291 mg, 0.72 mmol) in 10 mL of anhydrous dichloromethane was treated with boron trifluoride-methyl sulfide (289 lL, 1.8 mmol) at 0° C. The mixture was stirred for 5 days at room temperature. The reaction monitored by TLC (EtOAc: 2% EtN₃N) and when complete the mixture was washed with water, brine, and the organic phase was dried with anhydrous magnesium sulfate, and concentrated. The product was isolated from the residue by column chromatography on silica gel eluted with 2% Et₃N in EtOAc to yield 6′-hydroxy-5-(2″-piperidinylethoxy)-1,3,3′,4′-tetrahydro-spiro[2H-indene-2,2′(1′H)-naphthalene]-1′-one. ¹H NMR (CDCl₆): ä 1.25 (m, 1H), 1.28-1.45 (m, 1H), 1.53 (m, 1H), 2.17 (m, 2H), 2.97 (m, 2H), 3.06-3.20 (m, ?), 3.43 (m, 2H), 3.5 (m, 2H), 3.73 (m, 2H), 4.45 (2H), 6.65 (dd, 1H), 6.71 (d, 1H), 6.79 (d, 1H), 6.88 (dd, 1H), 7.07 (d, 1H), 7.93 (d, 1H). LC-MS; 392.2, LC MS: 390.4.

### EXAMPLE 49

### A pharmaceutical formulation comprising 5,5′-Dihydroxy-1-propyl-3-methyl-1,1,3,3′-tetrahydro-2,2′-spirobi(H-indene)

32 mg of 5,5′-Dihydroxy-1-propyl-3-methyl-1,1′,3,3′-tetrahydro-2,2′-spirobi(2H-indene), from Example 20, is formulated with sufficient finely divided lactose to provide a total amount of 580 to 590 mg to fill a size 0, hard-gelatine capsule.

### Description of the Scintistrip ER Binding Assay

Introduction

The scintistrip assay differs from a traditional hormone binding assay by not requiring the removal of free tracer prior to the measurement of receptor bound tracer. The scintillating agent is in the polystyrene forming the incubation vial and thus a radioactive molecule in the close proximity to the surface will induce scintillation of the plastic. For ³[I]-labeled ligands, the distance between the free tracer and the scintillating polystyrene surface is too far to induce scintillation of the plastic while ³[H]-labeled ligands bound to receptors immobilized on the surface are close enough to induce scintillation thus enabling a convenient way to measure the competition between a non-radioactive estrogen receptor interacting agent (the compound to be tested) and a fixed concentration of tracer (³[H]-Estradiol).

Materials and Methods

³[H]-β-Estradiol (NET 317) hereafter referred to as ³[H]-E2 was purchased from New England Nuclear, Boston, Mass. The scintistrip wells (1450-419) and the scintillation counters (Microbeta™ 1450-Plus and 1450-Trilux) were all from Wallac, Turku, Finland. Human estrogen receptors (hER) alpha and beta were extracted from the nuclei from SF9-cells infected with a recombinant baculovirus transfer vector containing the cloned hER genes. Recombinant baculovirus was generated utilizing the BAC-TO-BAC expression stem (Life Technologies) in accordance to instruction from the supplier. The hER coding sequences were cloned into a baculovirus transfer vector by standard techniques. The recombinant baculoviruses expressing hER were amplified and used to infect SF9 cells. Infected cells were harvested 48 hr post infection. A nuclear fraction was obtained as described in² and the nuclei were extracted with a high-salt buffer (17 mM K₂HPO₄, 3 mM KH₂PO₄, 1 mM MgCl₂, 0.5 mM EDTA, 6 mM MTG, 400 mM KCl, 8.7% Glycerol). The concentration of hER's in the extract was measured as specific [³H]-E2 binding with the G25-assay³ and was determined to contain 400 pmols specific bound [3H]-E2/mL nuclear extract in the case of hER-alpha and 1000 pmols/mL nuclear for hER-beta. The total concentration of proteins (as determined with Bradford Reagent, Bio-Rad according to instructions from manufacturer) in the nuclear extracts were 2 mg/mL. The equilibrium binding constant (Ks) for pM-E2 to hER in solution was determined to 0.05 nM for hER-alpha and to 0.07 nM for hER-beta with the G25-assay for highly diluted extracts (hER ^{˜}0.1 nM). The extracts were aliquoted and stored at −80° C.

The scintistrip assay¹ In brief; the nuclear extracts were diluted (50 fold for hER-alpha and 110 fold for hER-beta) in coating buffer (17 mM K₂HPO₄, 3 mM KH₂PO₄, 40 mM KCl, 6 mM MTG). The diluted extracts were added to Scintistrip wells (200 μL/well) and incubated 18-20 hr. at ambient room temperature (22-25° C.). The estimated final concentration of immobilize hER in all experiments was ^{˜}nM. All incubations were performed in 17 mM K₂HPO₄, 3 mM KH₂PO₄, 140 mM KCl, 6 mM MTG (buffer A). The wells were washed twice after hER coating with 250 μL buffer prior to addition of the incubation solution. All steps were carried out at ambient room temperature (22-25° C.).

Determination of Equilibrium binding constants to immobilized hER:s Dilutions of ³[H]-E2, in buffer±Triton X100 were added to the wells (200 μL/well), the wells were incubated for 3 hr and then measured in the Microbeta After the measurement an aliquot of the buffer was taken out and counted by regular liquid scintillation counting for determination of the “free” fraction of ³[H]-E2. In order to correct for non-specific binding parallel incubations were done in presence of a 200-fold excess of unlabeled 17-β-E2. The equilibrium dissociation constants (K_{d}) were calculated as fee concentration of ³[H]-E2 at half maximum binding by fitting data to the Hill equation; b=(bₘₐₓ×Lⁿ)/Lⁿ+K_{d}ⁿ),where b is specific bound ³[H]-E2, bₘₐₓ is the maximum binding level, L is the free concentration of [³H]E2, n is the Hill coefficient (the Hill equation equals the Michaelis-Menten equation when n=1). The equilibrium binding constants were deter ed to 0.15-0.2 nM for both hER subtypes.

Regular competition binding. Samples containing 3 nM [3H]-E2 plus a range of dilutions of the compounds to be tested were added to wells with immobilized hER and incubated for 18-20 hr at ambient room temperature. The compounds to be tested were diluted in 100% DMSO to a concentration 50 fold higher than the desired final concentration, the final concentration of DMSO was thus 2% in all samples. For compounds able to displace ³[H]-E2 from the receptor an IC₅₀-value (the concentration required to inhibit 50% of the binding of ³[H]-E2) was determined by a non-linear four parameter logistic model; b=((bₘₐₓ−bₘᵢₙ)(1+(I/IC₅₀)^{s}))+bₘᵢₙ is added concentration of binding inhibitor, IC₅₀ is the concentration of inhibitor at half maximal binding and S is a slope factor.¹ For determinations of the concentration of ³[H]-E2 in the solutions regular scintillation counting in a Wallac Rackbeta 1214 was performed using the scintillation cocktail Supermix™ (Wallac).

The Microbeta-instrument generates the mean cpm (counts per minute) value/minute and corrects for individual variations between the detectors thus generating corrected cpm values. It was found that the counting efficiency between detectors differed with less than five percent.

1) Haggblad, J., Carlsson, B., Kivelä, P., Siitari, H., (1995) Biotechniques 18, 146-151

2) Barkhem, T., Carlsson, B., Simons, J., Moller, B., Berkenstam A., Gustafsson J.A.G., Nilsson, S. (1991) J. Steroid Biochem Molec. Biol 38, 667-75

3) Salomonsson, M., Carlsson, B., Haggblad, J., (1994) J. Steroid Biochem. Molec. Biol. 50,313-318

4) Schultz, J. R, Ruppel, P. L, Johnson, M. A., (1988) in Biopharmaceutical Statistics for Drug Development (Peace, K. E., Ed.) pp. 21-82, Dekker, New York

The compounds of Examples 1-48 exhibit binding affinities to the estrogen receptor α-subtype in the range of IC₅₀ 3 to 10,000 nM and to the estrogen receptor β-subtype in the range of IC₅₀ 3 to 10,000 nM.

1. A compound having the general formula I: 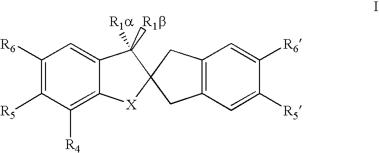
wherein R₁α and R₁β may together be a single nitrogen atom which is in turn bonded to a group selected from R^{A} or OR^{A} or R₁α and R₁β may together be a single carbon atom which in turn is bonded to two R^{A} groups which may be the same or are different; or R₁α and R₁β are the same or are different and selected from the group R^{A} or OR^{A},
R^{A} is selected from the group, hydrogen, alkyl alkenyl, alkynyl, cycloalkyl, cycloalkylalkyl, aryl or arylalkyl,
provided that R₁α and R₁β are not both R₁α is not OH when R₁β is H, and R₁β is not OH when R₁α is H,
X is a methylene group (—CH₂—), an ethylene group (—CH₂CH₂—), or a substituted methylene group (—CH_{B}—) where R^{B} is a alkyl group of from 1 to 4 carbon atoms,
R₄ is a hydrogen atom, or an alkyl group of from 1 to 4 carbon atoms, or a halogen atom,
R₅, R₆, R_{5′}, and R_{6′} are the same or are different and are selected from the group hydrogen, halogen, hydroxyl, alkyloxy, acyloxy, or aminoalkoxy,
and pharmaceutically acceptable salts and stereoisomers thereof.

2. A compound according to claim 1 wherein at least one of the R₅ or R₆ substituents is a hydrogen atom and at least one of the R_{5′} or R_{6′} substituents is also a hydrogen atom.

3. A compound according to claim 2 wherein at least one of R₆, R₆, R_{5′}, or R_{6′} is a group selected from hydroxyl, acyloxy, chlorine, or bromine.

4. A compound according to claim 2 wherein the remaining substituents R₅, R₆, R_{5′}, or R_{6′} are the same or are different and selected from the group hydroxyl or acyloxy.

5. A compound according to claim 2 wherein one of the remaining substituents R₅, R₆, R_{5′}, or R_{6′} is hydroxyl or acyloxy and the other remaining substituent is aminoalkoxy as herein defined.

6. A compound according to any one of claims 1 to 5 wherein one of R₁α and R₁β is selected from the group hydrogen or methyl or hydroxyl and the other is selected from the group n-propyl 2-propenyl, 2-propynyl, n-butyl, 2-butenyl, 3-butenyl, 2-butynyl, 3-butynyl, n-pentyl, 3-methylbutyl, 3-methyl-1-butenyl, 3-methyl-2-butenyl, 3-methylpentyl, 3-ethylpentyl, cyclopropylethyl, cyclopentylethyl, cyclohexylethyl, cycloheptylethyl, cyclopropylpropyl, cyclopentylpropyl, benzyl, or phenethyl.

7. A compound according to any one of claims 1 to 5 wherein R₁α and R₁β may together be a single carbon atom (i.e., an exo methylene carbon atom) which in turn is bonded to two groups R^{C} and R^{D}, wherein R^{C} is selected from the group hydrogen or methyl and R^{D} is selected from the group aryl, benzyl, ethyl, n-propyl, i-propyl, 2-propenyl, 2-propynyl, n-butyl, 2-butenyl, 3-butenyl, 2-butynyl, 3-butynyl, 2-methylbutyl, 2-methyl-1-butenyl, 2-methyl-2-butenyl, cyclopropylmethyl, cyclopentylmethyl, cyclohexylmethyl, or cycloheptylmethyl.

8. A compound according to claim 6 or claim 7 wherein X is a methylmethylene group [—C(CH₃)H—].

9. A compound having the general formula II or III: 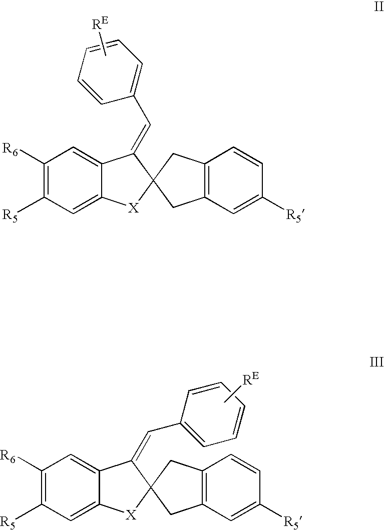
wherein X is a methylene group (—CH₂—) or an ethylene group (—CH₂CH₂—), one of R₅ or R₆ is a hydrogen atom and the other is a hydroxyl or acyloxy group, one of R_{5′} and R^{E} is selected from the group hydroxyl, acyloxy, methoxy, or ethoxy and the other is selected from the group aminoalkoxy;
and pharmaceutically acceptable salts and stereoisomers thereof.

10. A compound according to claim 1, which is:
Anti-5,5′-dihydroxy-1,1′,3,3′-tetrahydro-2,2′-spirobi(2H-indene)-1-one-(N-methyl oxime) (E9a);
Syn-5,5′-dihydroxy-1,1′,3,3′-tetrahydro-2,2′-spirobi(2H-indene)-1-one-(N-methyl oxime)(E9b);
5,5′-Dihydroxy-1,1′,3,3′-tetrahydro-2,2′-spirobi(2H-indene)-1-one-oxime (E10a);
5′-Hydroxy-5-methoxy-1,1′,3,3′-tetrahydro-2,2′-spirobi(2H-indene)-1-one-oxime (E10b);
5,5′-Dihydroxy-1,1′,3,3′-tetrahydro-2,2′-spirobi(2H-indene)-1-methylidene (E11);
5,5′-Dihydroxy-1-methyl-1,1′,3,3′-tetrahydro-2,2′-spirobi(2H-indene) (E12);
1-Butyl-5,5′-hydroxy-1,1′,3,3′-tetrahydro-2,2′-spirobi(2H-indene) (E13);
5-Hydroxy-5′-(2′-piperidinylethoxy)-1(p-methoxy)benzylidene-1,1′,3,3′-tetrahydro-2,2′-spirobi(2H-indene) (E14a);
6-Hydroxy-5′-(2″-piperidinylethoxy)-1-(p-methoxy)benzylidene-1,1′,3,3′-tetrahydro-2,2′-spirobi(2H-indene) (E14b);
Z-5-Hydroxy-5′-(2″-piperidinylethoxy)-1-(m-methoxy)benzylidene-1,1′,3,3′-tetrahydro-2,2′-spirobi(2H-indene) (E14c);
Z-5-Hydroxy-5′-(2″-piperidinylethoxy)-1-(m-hydroxy)benzylidene-1,1′,3,3′-tetrahydro-2,2′-spirobi(2H-indene) (E14d);
5,5′-Dihydroxy-1,3-dimethyl-1,1′,3,3′-tetrahydro-2,2′-spirobi(2H-indene) (E18);
5,5′-Dihydroxy-1-ethyl-3-methyl-1,1′,3,3′-tetrahydro-2,2′-spirobi(2H-indene) (E19);
5,5′-Dihydroxy-1-propyl-3-methyl-1,1′,3,3′-tetrahydro-2,2′-spirobi(2H-indene) (E20);
6,5′-Dihydroxy-1-methyl-1,1′,3,3′-tetrahydro-2,2′-spirobi(2H-indene) (E23);
6,5′-Dihydroxy-1-ethyl-1,1′,3,3′-tetrahydro-2,2′-spirobi(2H-indene) (E24);
6,5′-Dihydroxy-1-butyl-1,1′,3,3′-tetrahydro-2,2′-spirobi(2H-indene) (E25);
6,5′-Dihydroxy-1-benzylidene-1,1′,3,3′-tetrahydro-2,2′-spirobi(2H-indene) (26);
6′,5′-Dihydroxy-1-(p-methoxy)benzylidene-1,1′,3,3′-tetrahydro-2,2′-spirobi(2H-indene) (E27);
6,5′-Dihydroxy-1-(p-hydroxy)benzylidene-1,1′,3,3′-tetrahydro-2,2′-spirobi(2H-indene) (E28);
Rac-(1′R,2S/1′S,2R)-6,5′-dihydroxy-1-(p-methoxy)benzyl-1,1,3,3′-tetrahydro-2,2′-spirobi(2H-indene) (E29a);
rac-(1′R,2R/1′S,2S)-6,5′-Dihydroxy-1-(p-methoxy)benzyl-1,1′,3,3′-tetrahydro-2,2′-spirobi(2H-indene) (E29b);
6,5′-Di[(t-butyldimethyl)silyloxy]-1-[4-benzyloxy(benzylidene)]-1,1′,3,3′-tetrahydro-2,2′-spirobi(2H-indene) (E30);
6,5′-Dihydroxy-1-(p-benzyloxy)benzylidene-1,1′,3,3′-tetrahydro-2,2′-spirobi(2H-indene) (E31);
rac-(1′R,2S/1′S,2R)-6,5′-Dihydroxy-1-[p-(2″-piperidinylethoxy)benzyl]-1,1′,3,3′-tetrahydro-2,2′-spirobi(2H-indene) (E32a);
rac-(1′R,2R/1′S,2S)-6,5′-Dihydroxy-1-[p-(2″-piperidinylethoxy)benzyl]-1,1′,3,3′-tetrahydro-2,2′-spirobi(2H-indene) (E32b);
5,7′-Dihydroxy-1′-methyl-1,3,3′,4′-tetrahydro-spiro[2H-indene-2,2′-(1H)-naphthalene] (E38);
5,6′-Dihydroxy-1′-methyl-1,3,3′,4′-tetrahydro-spiro[2H-indene-2,2′-(1′H)-naphthalene] (E44);
5,6′-Dihydroxy-1′-ethylidene-1,3,3′,4′-tetrahydro-spiro[2H-indene-2,2-(1′H)-naphthalene] (E45a);
5,6 ′-Dihydroxy-1′-isopropylidene-1,3,3′,4′-tetrahydro-spiro[2H-indene-2,2′-(1′H)-naphthalene] (E45b);
(Z)-5,6′-Dihydroxy-1′-propylidene-1,3,3′,4′-tetahydro-spiro[2H-indene-2,2′-(1′H)-naphthalene] (E45c);
(E)-5,6′-Dihydroxy-1′-propylidene-1,3,3′,4′-tetrahydro-spiro[2H-indene-2,2′-(1′H)-naphthalene] (E45d);
(1R,2S)-and (1S,2R)-5,1′,6′-Trihydroxy-1′-phenyl-1,3,3′,4′-tetrahydro-spiro[2H-indene-2,2′-(1′H)-naphthalene] (E45e);
(1S,2S)-5,1′,6′-Trihydroxy-1′-phenyl-1,3,3′,4′-tetrahydro-spiro[2H-indene-2,2′-(1′H)naphthalene] (E45f);
5,6′-Dihydroxy-1′-(p-methoxy)benzylidene-1,3,3′,4′-tetrahydro-spiro[2H-indene-2,2′-(1′H)-naphthalene] (E46);
and pharmaceutically acceptable salts and stereoisomers thereof.

11. A compound according to any one of claims 1 to 10 for use in medical therapy.

12. A pharmaceutical composition comprising a compound according to any of the claims 1-10 and a pharmaceutically acceptable carrier.

13. A process for making a pharmaceutical composition comprising combining a compound according to any one of claims 1 to 10 and a pharmaceutically acceptable carrier.

14. A method of eliciting an estrogen receptor modulating effect in a mammal in need thereof; comprising a to the mammal a therapeutically effective amount of a compound according to any one of claims 1 to 10.

15. The method according to claim 14 wherein the estrogen receptor modulation effect is an estrogen receptor agonizing effect.

16. The method according to claim 15 wherein the estrogen receptor agonizing effect is an ERα receptor agonizing effect.

17. The method according to claim 15 wherein the estrogen receptor agonizing effect is an ERβ receptor agonizing effect.

18. The method according to claim 15 wherein the estrogen receptor agonizing effect is a mixed ERα and ERβ receptor agonizing effect.

19. The method according to claim 14 wherein the estrogen receptor modulation effect is an estrogen receptor antagonizing effect.

20. The method according to claim 19 wherein the estrogen receptor antagonizing effect is an ERα receptor antagonizing effect.

21. The method according to claim 19 wherein the estrogen receptor antagonizing effect is an ERβ receptor antagonizing effect.

22. The method according to claim 19 wherein the estrogen receptor antagonizing effect is a mixed ERα and ERβ receptor antagonizing effect.

23. A method of treating or preventing a disease regulated by the estrogen receptor in a mammal in need thereof by administering to the mammal a therapeutically effective amount of a compound according to any one of claims 1 to 10.

24. A method of treating or preventing bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restinosis, gynecomastia, vascular smooth muscle cell proliferation, obesity, incontinence, autoimmune disease, and lung, colon, breast, uterus, and prostate cancer in a mammal in need thereof by administering to the mammal a therapeutically effective amount of a compound according to any one of claims 1 to 10.

25. The use of a compound according to any one of claims 1 to 10 in the manufacture of a medicament for the therapeutic treatment or prevention of bone loss, bone fractures, osteoporosis, cartilage degeneration, endometriosis, uterine fibroid disease, hot flashes, increased levels in LDL cholesterol, cardiovascular disease, impairment of cognitive functioning, cerebral degenerative disorders, restinosis, gynecomastia, vascular smooth muscle cell proliferation, obesity, incontinence, autoimmune disease, and lung, colon, breast, uterus and prostate cancer.
